(19) United States
(12) Patent Application Publication
(10) Pub. No.: US 2026/0327648 A1
(43) Pub. Date: Oct. 22, 2026

(54) Envelope Proteins

(71) Applicant: Oxford Biomedica (UK) Limited, Oxford (GB)

(72) Inventors: Rachael Nimmo, Oxford (GB); Tiziana Coradin, Oxford (GB); Anurag Kulkarni, Oxford (GB)

(21) Appl. No.: 19/104,214

(22) PCT Filed: Aug. 15, 2023
(86) PCT No.: PCT/GB2023/052145

§ 371 Date: Feb. 15, 2025

(30) Foreign Application Priority Data
Aug. 16, 2022 (GB) 2211935.8

(51) Int. Cl. C12N 15/86; C07K 14/005; C07K 16/28

(52) U.S. Cl. CPC C12N 15/86; C07K 14/005; C07K 16/2815; C07K 2317/622; C12N 2760/18222; C12N 2760/18245; C12N 2760/18251; C12N 2760/20222

(57) ABSTRACT

The invention relates to a viral vector production system. More specifically, the present invention relates to a set of nucleotide sequences comprising sequences encoding heterologous envelope proteins that facilitates the production of mixed envelopes, wherein the mixed envelopes comprise a mixed species of envelope proteins. Viral vectors, methods and uses of such viral vector production systems are also encompassed by the invention.

### FIELD OF THE INVENTION

The invention relates to a viral vector production system and viral vectors produced thereby, which comprise heterologous envelope proteins. More specifically, the invention relates to a viral vector production system and viral vectors comprising heterologous envelope proteins that may target specific cell types and reduce or eliminate off-target transduction. Further provided is a viral vector production system and viral vectors that comprise a mixed envelope, which may increase transduction efficiency of viral vectors. Methods and uses involving such a viral vector production system and viral vectors are also encompassed by the invention.

### BACKGROUND TO THE INVENTION

The development and manufacture of viral vectors towards vaccines and human gene therapy over the last several decades is well documented in scientific journals and in patents. The use of engineered viruses to deliver transgenes for therapeutic effect is wide-ranging. Contemporary gene therapy vectors based on RNA viruses such as γ-retroviruses and lentiviruses (Muhlebach, M. D. et al., 2010, Retroviruses: Molecular Biology, Genomics and Pathogenesis, 13:347-370; Antoniou, M. N., Skipper, K. A. & Anakok, O., 2013, Hum. Gene Ther., 24:363-374), and DNA viruses such as adenovirus (Capasso, C. et al., 2014, Viruses, 6:832-855) and adeno-associated virus (AAV) (Kotterman, M. A. & Schaffer, D. V., 2014, Nat. Rev. Genet., 15:445-451) have shown promise in a growing number of human disease indications. These include ex vivo modification of patient cells for hematological conditions (Morgan, R.A. & Kakarla, S., 2014, Cancer J., 20:145-150; Touzot, F. et al., 2014, Expert Opin. Biol. Ther., 14:789-798), and in vivo treatment of ophthalmic (Balaggan, K. S. & Ali, R. R., 2012, Gene Ther., 19:145-153), cardiovascular (Katz, M. G. et al., 2013, Hum. Gene Ther., 24:914-927), neurodegenerative diseases (Coune, P. G., Schneider, B. L. & Aebischer, P., 2012, Cold Spring Harb. Perspect. Med., 4: a009431) and tumour therapy (Pazarentzos, E. & Mazarakis, N. D., 2014, Adv. Exp. Med Biol., 818:255-280).

Viruses typically comprise envelope proteins that mediate cellular attachment and fusion of the viral and target cell envelopes. These attachment and fusion functionalities may be performed by a single protein (e.g., in VSV, influenza or Lassa virus) or by separate dedicated proteins (e.g., in paramyxoviruses such as Nipah virus, Hendra virus, Measles virus, and Newcastle disease virus), which work in concert to mediate target cell recognition and viral entry. Viruses may be ‘pseudotyped’ to display the envelope proteins from another virus. Pseudotyping may be utilised to take advantage of the functional properties of the heterologous envelope proteins, such as the ability to target new cellular receptors and therefore new cell types.

Lentiviral vectors typically consist of human immunodeficiency virus (HIV-1) or equine infectious anaemia virus (EIAV) particles that have been pseudotyped with the envelope G protein derived from vesicular stomatitis virus (VSV-G). The broad tissue tropism of VSV-G permits transduction of the lentiviral vector in a wide range of cell types. However, the VSV-G receptor (LDL-R) is ubiquitously expressed, making it unsuitable as a receptor for precision targeting. Identification of tissue specific markers and ligands that bind to them can be used to limit the transduction to the target cell type, eliminating transduction of off-target tissues, thereby improving overall safety when using lentiviral vectors.

Viral vectors pseudotyped with envelope proteins from Nipah virus (NiV) have been described. Said pseudotypes comprise both NiV envelope proteins that retain their endogenous specificity for ephrin B2 and ephrin B3, and proteins that have been modified to ablate said specificity. Envelope proteins may further be modified to ‘retarget’ them to an alternate receptor by providing specificity for said receptor. Whilst heterologous envelopes may improve target specificity, they can be associated with reduced transduction efficiency.

In view of the above, there is a need in the art for viral vectors with improved safety profiles that arise as a result of reliable and specific targeting of desired cell types that retain said effects without suffering from undesirably low transduction efficiencies, which may be associated with pseudotyped envelope proteins and modified pseudotyped envelope proteins.

### SUMMARY OF THE INVENTION

The present invention relates to viral vectors, such as lentiviral vectors (LVs), with improved safety, target specificity and transduction efficiency.

In a first aspect, there is provided a viral vector production system comprising a set of nucleotide sequences, wherein the nucleotide sequences encode heterologous envelope proteins comprising at least one target binding protein and at least one viral envelope protein.

In a second aspect, there is provided a viral vector production system comprising a set of nucleotide sequences, wherein the nucleotide sequences encode heterologous envelope protein sequences comprising at least one viral attachment protein, wherein the nucleotide sequences separately encode:
(i) a non-retargeted viral attachment protein; and
(ii) a retargeted viral attachment protein.

In a further aspect, there is provided a cell comprising the viral vector production system according to the invention.

In a further aspect, there is provided a cell for producing viral vectors comprising the viral vector production system according to the invention.

In a further aspect, there is provided a method for producing a viral vector, comprising the steps of:
(a) introducing the viral vector production system according to the invention into a cell;
(b) optionally, selecting for a cell which comprises the nucleotide sequences of the viral vector production system according to the invention; and
(c) culturing the cell under conditions suitable for the production of the lentiviral vector.

In a further aspect, there is provided a viral vector produced by a method according to the invention.

In a further aspect, there is provided the use of the viral vector production system or the cell according to the invention, for producing a viral vector

In a further aspect, there is provided a lentiviral vector produced according to the use according to the invention.

It is intended that one or more of the aspects of the viral vector production system of the invention may be combined with any viral vector components as described herein. Any viral vector may be pseudotyped using the envelope proteins described herein. It is also intended that one or more of the aspects of the invention may be combined during the production of the same viral vector.

### DESCRIPTION OF THE FIGURES

FIG. 1. Schematic representation of retargeted and non-retargeted viral attachment proteins.

Schematic representation depicting the overall domain organisation of an exemplary retargeted attachment protein and an exemplary non-retargeted attachment protein, such as a paramyxovrus attachment protein. Retargeted and non-retargeted viral attachment proteins differ by the presence or absence of a retargeting moiety in the ectodomain.

FIG. 2. Specificity and improved transduction efficiency of vectors pseudotyped with different ratios of envelope proteins.

Vectors pseudotyped with a blinded Nipah G protein (NiV-GmA34h) that has been retargeted to bind CD8+ cells using a DARPin specific to CD8 and a modified Nipah F protein (FΔ22a) showed specific transduction of CD8+ cells (bottom left panel) whereas vectors pseudotyped with VSV-G (top left panel) lacked the target specificity of the NiV-GmA34 h/F pseudotyped vectors as they also transduced cells not expressing CD8 (CD8-cells). Vectors pseudotyped with a mixed envelope composition (a mixture of retargeted and non-retargeted NiV-GmA34h, in addition to the Nipah FΔ22a protein, bottom right panel) exhibited improved transduction efficiency over non-mixed envelopes (bottom left panel) utilising the same type of retargeted NiV-GmA34 h protein, and retained specificity for the CD8+ T cell population. In this example a ratio of 0.75:0.25 retargeted NiV-GmA34 h protein to non-retargeted GmA34 h was used.

FIG. 3. Improved transduction efficiency of vectors pseudotyped with different ratios of envelope proteins.

Transduction efficiency of lentiviral vectors pseudotyped with different envelope compositions, including mixed and non-mixed envelopes. Improved transduction efficiency is shown as fold increase in viral titre as calculated from the proportion of GFP+ CD8+ cells among a CD8+ population measured on day 12 post transduction with the relevant vector. Data account for the absolute number of CD8+ cells present at transduction. For data represented by the left two bars an scFV specific to CD8 was used as retargeting moiety, while for the right two bars a DARPin specific to CD8 was used. The mixed envelope composition with a ratio of retargeted NiV-GmA34 h to non-retargeted NiV-GmA34 h of 0.75:0.25 produced 5-fold higher titer on CD8+ T cells than the retargeted NiV-GmA34 h alone.

FIG. 4. Improved transduction efficiency of the vectors pseudotyped with a combination of re-targeted NiV G, Non-re-targeted NIV G and VSV-G K47Q envelope glycoproteins

FIG. 5. Transduction efficiency of lentiviral vectors pseudotyped with different ratios of mixed and non-mixed envelopes. The transduction efficiency is shown as percentage of GFP+ cells amongst the CD8+ T cell population measured at day 12 post transduction. Multiple ratios of retargeted NiV-GmΔ34 h CD8 DARPin to non-retargeted NiV-GmΔ34 h are shown, including 1.00:0.00, 0.875:0.125, 0.75:0.25, 0.625:0.375, 0.50:0.50, 0.25:0.75 and 0.00:1.00. The highest transduction efficiency was observed with ratios 0.75:0.25, 0.625:0.375 and 0.50:0.50, showing a 5-fold improvement compared to the 1.00:0.00 ratio (containing only NiV-GmΔ34 h CD8 DARPin). No transduction was observed in the CD8-population (grey bars).

FIG. 6. Improved transduction efficiency of CD8 targeting vectors expressing FMC63 CAR as transgene and pseudotyped with different ratios of envelope proteins

The transduction efficiency is shown as percentage of CAR+ cells amongst the CD8+ T cell population measured at day 10 post transduction. Vectors pseudotyped with a mixed envelope composition of 0.75:0.25 retargeted NiV-GmΔ34 h protein to non-retargeted GmΔ34h exhibited improved transduction efficiency over non-mixed envelopes utilising the same type of retargeted NiV-GmΔ34 h protein.

FIG. 7. Improved transduction efficiency of CD3 targeting vectors expressing FMC63 CAR as transgene and pseudotyped with different ratios of mixed and non-mixed envelopes.

The transduction efficiency is shown as percentage of FMC63 CAR+ cells amongst the CD3+ T cell population measured at day 12 post transduction. Multiple ratios of retargeted NiV-GmΔ34 h CD3 DARPin to non-retargeted NiV-GmΔ34 h are shown, including 1.00:0.00, 0.875:0.125, 0.75:0.25, 0.625:0.375, 0.50:0.50, 0.375:0.625, 0.25:0.75 and 0.00:1.00. The highest transduction efficiency was observed with ratio 0.50:0.50, showing a 3.5-fold improvement compared to the 1.00:0.00 ratio (containing only NiV-GmΔ34 h CD3 DARPin).

### DETAILED DESCRIPTION OF THE INVENTION

The invention relates to viral vectors and viral vector production systems for producing such vectors.

It will be understood that “viral vector” e.g., as used alone and in the context of “viral vector production system”, may refer to any suitable viral vector. A preferable viral vector to which the disclosures herein apply is a lentiviral vector.

### Envelopes and Pseudotyping

Enveloped viruses and viral vectors, such as lentiviral vectors, rely on viral envelope-(or membrane-) displayed proteins to recognise target cells for infection, and subsequently enter those cells in order to continue the viral lifecycle.

Viruses or viral vectors may have their endogenous envelope proteins replaced by other proteins with analogous function. Most commonly, this involves replacement with envelope proteins from other, heterologous, viruses in a process called pseudotyping. Pseudotyped viruses may obtain some characteristics of the heterologous virus from which the pseudotyped envelope proteins are derived, such as cell type specificity, and lose characteristics associated with their endogenous envelope proteins. This principle applies equally to vectors that are pseudotyped with non-viral proteins, such as the target binding proteins of the invention, wherein the binding specificity of said target binding proteins will direct the cell type specificity of the vector.

For viruses that utilise distinct attachment and fusion proteins, it may be advantageous that the heterologous envelopes comprise both the fusion and attachment protein from the same virus. Thus, in one embodiment, the attachment and fusion proteins are from the same virus.

In one embodiment, the attachment and fusion protein may be from different viruses.

Viral vectors according to the invention are pseudotyped. Pseudotyping can confer one or more advantages. For example, in the case of lentiviral vectors, the env gene product of the HIV based vectors would restrict these vectors to infecting only cells that express CD4. However, replacing the env gene in these vectors with ‘env’ sequences from other enveloped viruses, i.e. other attachment proteins, may afford the viral vector a broader infectious spectrum (Verma and Somia (1997) Nature 389 (6648): 239-242). By way of example, an HIV based vector has been pseudotyped with the glycoprotein from VSV (Verma and Somia (1997) Nature 389 (6648): 239-242). Accordingly, alternative sequences which perform the equivalent function as the env gene product of HIV based vectors are also known.

In another alternative, the Env protein may be a modified Env protein such as a mutant or engineered Env protein. Modifications may be made or selected to introduce targeting ability

or to reduce toxicity or for another purpose (Valsesia-Wittman et al 1996 J Virol 70:2056-64; Nilson et al (1996) Gene Ther 3 (4): 280-286; and Fielding et al (1998) Blood 91 (5): 1802-1809; Frank AM & Buchholz CJ. Mol Ther Methods Clin Dev. 2018 Oct. 17; 12:19-31 and references cited therein).

The vector may be pseudotyped with any molecule of choice.

As used herein, “env” or envelope protein(s), such as viral attachment proteins or non-viral target-binding molecule, shall mean an endogenous lentiviral envelope or an heterologous envelope, as described herein.

### Receptor Binding Proteins

According to the first aspect of the invention, there is provided a viral vector production system comprising a set of nucleotide sequences, wherein the nucleotide sequences encode heterologous envelope proteins comprising at least one target binding protein and at least one viral envelope protein.

By the term “target binding protein” it is meant any protein, be it naturally occurring or synthetic, that has affinity for one or more receptor or ligand such that it is able to bind said receptor or ligand and confer specificity for a cell expressing the same. The target binding protein is able to determine the specificity or cellular tropism of the viral vector. Target binding proteins are not necessarily viral or virus-derived proteins.

In one embodiment, the target binding protein is a non-viral protein.

In one embodiment, the target binding protein is a viral protein.

In one embodiment, the target binding protein is a viral attachment protein.

Target binding proteins may comprise chimeric proteins that capitalise on the functionalities of one or more different polypeptide sequences or domains. For example, a suitable chimeric protein may comprise an extracellular (or extraviral) domain that has target binding functionality and a transmembrane domain that would allow virion membrane integration.

In one embodiment, the target binding protein is a chimeric protein.

Target binding proteins may be modified, e.g., by chemical modification, to engender target specificity. For example, a target binding protein may be modified to comprise a ligand for a known receptor (or vice versa).

In one embodiment, the target binding protein is chemically modified.

In one embodiment, a fraction of the target binding proteins are chemically modified.

Target binding proteins according to the invention may be present in envelopes alongside viral fusion proteins. These two types of proteins may function analogously to viral envelope proteins (e.g., attachment and fusion proteins) by facilitating target cell binding and subsequent cellular entry.

Viral fusion proteins catalyse the merger of viral and cellular membranes, allowing the release of the viral genetic payload into target cells. Viral fusion proteins may require environmental stimuli to trigger fusion, e.g., pH change or ligand binding. However, some viral fusion proteins may be spontaneously triggered, or be triggered in the absence of their usual stimulus.

In one embodiment, the viral envelope protein according to the first aspect of the invention is a fusion protein.

Some viral fusion proteins also constitute attachment proteins insofar as they contain a receptor-binding attachment domain (e.g., VSV-G). In such instances, it may be beneficial to modify said protein in order to ablate endogenous receptor specificity so that it does not interfere with the cellular targeting mediated by the receptor binding protein.

In one embodiment, the viral envelope protein is receptor-blinded.

Envelope proteins may suitably comprise polypeptide tags. Polypeptide tags are known in the art, e.g., His- (such as His₆ or His₈) and FLAG-tags, and may be used to, for example, increase protein expression, aid purification, and/or to allow detection.

### Mixed Envelopes

The present inventors surprisingly found that a viral vector production system comprising a set of nucleotide sequences that encode heterologous envelope protein sequences that facilitate the production of viral vectors containing ‘mixed envelopes’, increases transduction efficiency over non-mixed envelopes, whilst retaining target specificity.

The mixed envelopes of the invention refer to viral envelopes, such as viral vector envelopes, in which a single envelope comprises at least two distinct species of attachment protein. The two species may be differentiated by the presence of a targeting moiety.

Thus, within the set of nucleotide sequences according to the invention, a fraction of said sequences may comprise two distinct versions of the same or similar attachment protein, such as versions distinguished by the presence (or absence) of a retargeting moiety. For example, if the attachment proteins are provided as plasmids, two distinct plasmids may be supplied in one viral vector production system such that two distinct protein products are incorporated into one viral vector.

Attachment proteins may be ‘blinded’ such that they have, by way of mutation or otherwise, their endogenous specificity altered or ablated. Such attachment proteins will not bind their endogenous receptors. Blinded attachment proteins may have their target specificity dictated by a targeting moiety.

In one embodiment, the attachment protein is receptor-blinded such that it does not bind to one or more of its endogenous receptors.

Viral attachment proteins may be ‘retargeted’. By “retargeted” it is meant that viral attachment proteins are modified, e.g., by mutation or the addition of a targeting moiety, to engender specificity for a target protein other than the native or endogenous receptor for which the attachment protein has specificity.

Thus, in an aspect there is provided a viral vector production system comprising a set of nucleotide sequences, wherein the nucleotide sequences encode heterologous envelope protein sequences comprising at least one viral attachment protein, wherein the nucleotide sequences separately encode:
(i) a non-retargeted viral attachment protein; and
(ii) a retargeted viral attachment protein.

By non-retargeted it is simply meant that an attachment protein has not been modified with a retargeting moiety. In other words, (i) and (ii) according to the foregoing may be distinguished by the presence of a retargeting moiety.

In one embodiment, the viral vector produced by the viral vector production system is a lentiviral vector.

In one embodiment, there is provided a viral vector production system comprising a set of nucleotide sequences, wherein the set of nucleotide sequences comprises both non-retargeted attachment protein and retargeted attachment protein sequences.

The ratio of attachment protein sequences in the viral vector production system may be varied.

As used herein, “attachment protein sequences” refers to the nucleotide sequences of the invention that encode said attachment proteins.

To generate mixed envelopes, it will be understood that the ratio of retargeted to non-retargeted attachment proteins may be any value except for 1:0 and 0:1, which would represent non-mixed envelopes.

In one embodiment, the ratio of non-retargeted attachment protein sequences to retargeted attachment protein sequences is within the range of 0:1 to 1:0, but is neither 0:1 or 1:0, such as 0.125:0.875, 0.25:0.75, 0.375:0.625, 0.5:0.5, or 0.75:0.25.

In one embodiment, the ratio of non-retargeted attachment protein sequences to retargeted attachment protein sequences is 0.125:0.875, 0.25:0.75, 0.375:0.625, 0.5:0.5, or 0.75:0.25.

In some embodiments, the ratio of non-retargeted attachment protein sequences to retargeted attachment protein sequences is does not include 0:1 and 1:0, i.e., non-mixed envelopes, but comprises any ratio that constitutes a mixed envelope. A mixed envelope may comprise a ratio of retargeted attachment protein sequences to non-retargeted attachment protein sequences within the range of 0.05:0.95 to 0.95:0.05, suitably from 0.125:0.875 to 0:75:0.25, preferably from 0.25:0.75 to 0.5:0.5. A mixed envelope may comprise a ratio of non-retargeted attachment protein sequences to retargeted attachment protein sequences of 0.05:0.95, 0.10:0.9, 0.125:0.875, 0.15:0.85, 0.20:0.80, 0.25:0.75, 0.30:0.70, 0.35:0.65, 0.375:0.625, 0.40:0.60, 0.45:0.55, 0.50:0.50, 0.55:0.45, 0.60:0.40, 0.65:0.35, 0.70:0.30, 0.75:0.25, 0.80:0.20, 0.85:0.15, 0.90:0.10, or 0.95:0.05.

In one embodiment, the ratio of non-retargeted attachment protein sequences to retargeted attachment protein sequences is from 0.05:0.95 to 0.95:0.05.

In one embodiment, the ratio of non-retargeted attachment protein sequences to retargeted attachment protein sequences is from 0.125:0.875 to 0.75:0.25.

In one embodiment, the ratio of non-retargeted attachment protein sequences to retargeted attachment protein sequences is from 0.25:0.75 to 0.5:0.5.

In one embodiment, the ratio of non-retargeted attachment protein sequences to retargeted attachment protein sequences is 0.05:0.95, 0.10:0.9, 0.125:0.875, 0.15:0.85, 0.20:0.80, 0.25:0.75, 0.30:0.70, 0.35:0.65, 0.375:0.625, 0.40:0.60, 0.45:0.55, 0.50:0.50, 0.55:0.45, 0.60:0.40, 0.65:0.35, 0.70:0.30, 0.75:0.25, 0.80:0.20, 0.85:0.15, 0.90:0.10, or 0.95:0.05.

In one embodiment, the ratio of non-retargeted attachment protein sequences to retargeted attachment protein sequences is 0.5:0.5.

In one embodiment, the ratio of non-retargeted attachment protein sequences to retargeted attachment protein sequences is from 0.375:0.625.

In one embodiment, the ratio of non-retargeted attachment protein sequences to retargeted attachment protein sequences is 0.25:0.75.

In one embodiment, the ratio of non-retargeted attachment protein sequences to retargeted attachment protein sequences is from 0.125:0.875.

It will be understood that the ratio of attachment protein sequences may refer to the mass or molar ratio of the nucleotides, e.g., plasmids, comprising said nucleotide sequence.

In one embodiment, the ratio of attachment protein sequences is a mass ratio of the nucleotides, e.g., plasmids, comprising said nucleotide sequence.

According to the foregoing, there is provided a viral vector produced by the viral vector production system of the invention, or a use or method of the invention, wherein the viral vector envelope comprises:
(i) a viral fusion protein;
(ii) a non-retargeted receptor-blinded attachment protein; and
(iii) a retargeted receptor-blinded attachment protein.

It will be understood that, in instances where a viral envelope protein comprises both fusion and attachment functionality, i.e., it is both a fusion and attachment protein, a single protein may be considered to comprise (i) and (ii) according to the foregoing.

In one embodiment, there is provided a viral vector wherein the ratio of non-retargeted attachment protein to retargeted attachment protein is within the range of 0:1 to 1:0, but is neither 0:1 nor 1:0, such as 0.25:0.75, 0.5:0.5, or 0.75:0.25. In one embodiment, there is provided a viral vector wherein the ratio of non-retargeted attachment protein to retargeted attachment protein is within the range of 0.05:0.95 to 0.95:0.05, suitably from 0.125:0.875 to 0:75:0.25, preferably from 0.25:0.75 to 0.5:0.5.

As opposed to the use of “attachment protein sequences” as detailed above, reference to the “attachment protein” denotes the polypeptides.

In one embodiment, there is provided a viral vector wherein the ratio of non-retargeted attachment protein to retargeted attachment protein is 0.05:0.95, 0.10:0.9, 0.125:0.875, 0.15:0.85, 0.20:0.80, 0.25:0.75, 0.30:0.70, 0.35:0.65, 0.375:0.625, 0.40:0.60, 0.45:0.55, 0.50:0.50, 0.55:0.45, 0.60:0.40, 0.65:0.35, 0.70:0.30, 0.75:0.25, 0.80:0.20, 0.85:0.15, 0.90:0.10, or 0.95:0.05.

In one embodiment, there is provided a viral vector wherein the ratio of non-retargeted attachment protein to retargeted attachment protein is 0.125:0.875, 0.25:0.75, 0.375:0.625, 0.5:0.5, or 0.75:0.25.

In one embodiment, there is provided a viral vector produced by the viral vector production system of the invention, wherein the viral vector comprises an envelope comprising:
(i) a viral fusion protein;
(ii) a non-retargeted receptor-blinded attachment protein; and
(iii) a retargeted receptor-blinded attachment protein.

In one embodiment, there is provided a viral vector wherein the ratio of non-retargeted receptor-blinded attachment protein to retargeted receptor-blinded attachment protein is within the range of 0:1 to 1:0, but does not include 0:1 and 1:0, such as 0.125:0.875, 0.25:0.75, 0.375:0.625, 0.5:0.5, or 0.75:0.25. In one embodiment, there is provided a viral vector wherein the ratio of non-retargeted receptor-blinded attachment protein to retargeted receptor-blinded attachment protein is within the range of 0.05:0.95 to 0.95:0.05, suitably from 0.125:0.875 to 0:75:0.25, preferably from 0.25:0.75 to 0.5:0.5.

In one embodiment, there is provided a viral vector wherein the ratio of non-retargeted receptor-blinded attachment protein to retargeted receptor-blinded attachment protein is 0.05:0.95, 0.10:0.9, 0.125:0.875, 0.15:0.85, 0.20:0.80, 0.25:0.75, 0.30:0.70, 0.35:0.65, 0.375:0.625, 0.40:0.60, 0.45:0.55, 0.50:0.50, 0.55:0.45, 0.60:0.40, 0.65:0.35, 0.70:0.30, 0.75:0.25, 0.80:0.20, 0.85:0.15, 0.90:0.10, or 0.95:0.05.

In one embodiment, there is provided a viral vector wherein the ratio of non-retargeted receptor-blinded attachment protein to retargeted receptor-blinded attachment protein is 0.125:0.875, 0.25:0.75, 0.375:0.625, 0.5:0.5, or 0.75:0.25.

### Exemplary Envelope Proteins

Viral vectors may be pseudotyped with suitable envelope proteins. Such envelope proteins may be native sequences or modified sequences, depending on application. Further, such proteins may be derived from any number of suitable viruses.

It will be understood that when referring to envelope proteins herein, it is also intended to refer to any nucleotide sequence encoding the same, such as a nucleotide sequence in a viral vector production system.

In one embodiment the envelope protein is a modified protein.

In one embodiment the envelope protein is modified so that it does not bind to one or more of its endogenous receptors, i.e., it is receptor-blinded.

In one embodiment, the envelope protein is a negative sense RNA virus protein, or a modified version thereof.

In one embodiment the envelope protein is a mononegavirus (i.e., of the order mononegavirales) envelope protein, or a modified version thereof.

In one embodiment, the envelope protein is a paramyxovirus protein, or a modified version thereof.

In one embodiment, the envelope protein is a henipavirus protein, or a modified version thereof.

In one embodiment, the envelope protein is a Nipah (NiV) protein, or a modified version thereof.

Different viruses employ different systems for mediating cellular attachment and entry. Whilst some encode dedicated and separate polypeptides or proteins for these functions, other viruses possess proteins that harbour both activities in a single protein or protein complex. For example, paramyxoviruses, such a Nipah virus encode separate attachment (G) and fusion (F) proteins, which are displayed on their viral envelope. NiV-G and NiV-F work in concert to facilitate viral entry into target cells.

Both “attachment” and “fusion” proteins, as used herein, have their normal meaning in the art of virology. That is, attachment protein will be understood to describe a viral envelope protein that has the native functionality of binding one or more cellular receptors. The term fusion protein will be understood to describe a viral protein that has the native functionality of mediating or catalysing membrane fusion. As described above, a single protein may be both an attachment and fusion protein, albeit that these functionalities may be distributed across different domains, or even different polypeptide chains within a complex. As such, any polypeptide, such as a fragment, or domain that preserves the above functionality may be utilised as an attachment or fusion protein according to the present invention.

It is also envisaged that proteins from different viruses may be utilised together in the practice of the invention. That is, where present, an attachment and fusion protein need not necessarily be derived from the same virus. Further, more than one type (i.e., from more than one virus) of attachment protein may be provided.

In one embodiment, the envelope protein is an attachment protein.

In one embodiment, the envelope protein is a modified attachment protein.

In one embodiment, the non-retargeted attachment protein and retargeted attachment protein are derived from the same virus.

In one embodiment, the attachment protein is a negative sense RNA virus envelope protein, or a modified version thereof.

In one embodiment the attachment protein is a mononegavirus (i.g., of the order mononegavirales) attachment protein, or a modified version thereof.

In one embodiment, the viral attachment protein is a paramyxovirus attachment protein, or a modified version thereof.

In one embodiment, the viral attachment protein is a henipavirus attachment protein, or a modified version thereof.

In one embodiment, the viral attachment protein is a Nipah virus attachment protein (NIV-G), or a modified version thereof.

In one embodiment, the viral attachment protein is a receptor-blinded Nipah virus attachment protein.

In one embodiment, the viral attachment protein is a truncated Nipah virus attachment protein.

In one embodiment, the receptor-blinded Nipah virus attachment protein is a mA34 mutant of the Nipah virus attachment protein.

In one embodiment, the receptor-blinded Nipah virus attachment protein is a mA34 h mutant of the Nipah virus attachment protein. The proteins mA34 and mA34 h are distinguished by the presence of a his tag, as indicated by ‘h’.

In one embodiment, the viral attachment protein comprises a polypeptide tag, such as a His-tag. In one embodiment, the viral attachment protein is a Nipah virus attachment protein that comprises a His-tag.

In another embodiment, the receptor-blinded Nipah virus attachment protein comprises a sequence according to SEQ ID NO: 1 or SEQ ID NO: 2.

In another embodiment, the Nipah virus attachment protein consists of a sequence according to SEQ ID NO: 1 or SEQ ID NO: 2.

NiV-G mΔ34 (SEQ ID NO: 1):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPFREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRENTCPAICAEGVYNDAFLIDRINWISAGVELDSNATAA
NPVFTVFKDNEILYRAQLASEDTNAQKTITNCFLLKNKIWCISLVEIYDT
GDNVIRPKLFAVKIPEQCT
NiV-G mΔ34h (SEQ ID NO: 2):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPEREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRFNTCPAICAEGVYNDAFLIDRINWISAGVFLDSNATAA
NPVFTVFKDNEILYRAQLASEDTNAQKTITNCFLLKNKIWCISLVEIYDT
GDNVIRPKLFAVKIPEQCTHHHHHH

In one embodiment, the Nipah virus attachment protein comprises a sequence with at least 75% sequence identity to SEQ ID NO: 1 or 2, or a fragment thereof.

In one embodiment, the Nipah virus attachment protein comprises a sequence with at least 80% sequence identity to SEQ ID NO: 1 or 2, or a fragment thereof.

In one embodiment, the Nipah virus attachment protein comprises a sequence with at least 85% sequence identity to SEQ ID NO: 1 or 2, or a fragment thereof.

In one embodiment, the Nipah virus attachment protein comprises a sequence with at least 90% sequence identity to SEQ ID NO: 1 or 2, or a fragment thereof.

In one embodiment, the Nipah virus attachment protein comprises a sequence with at least 95% sequence identity to SEQ ID NO: 1 or 2, or a fragment thereof.

In one embodiment, the Nipah virus attachment protein comprises a sequence with at least 99% sequence identity to SEQ ID NO: 1 or 2, or a fragment thereof.

In one embodiment, the nucleotide sequences encode a viral fusion protein.

In one embodiment, the attachment protein is a rhabdovirus attachment protein, or a modified version thereof.

In one embodiment, the attachment protein is a vesicular stomatitis virus (VSV) attachment protein (VSV-G), or a modified version thereof.

In another embodiment, the attachment protein comprises a sequence according to SEQ ID NO: 34 or SEQ ID NO: 35.

In another embodiment, the attachment protein consists of a sequence according to SEQ ID NO: 34 or SEQ ID NO: 35.

In one embodiment, the viral attachment protein:
(a) is a K47Q mutant of VSV-G;
(b) is a R354A mutant of VSV-G; and/or
(c) comprises a sequence of SEQ ID NO: 34 or SEQ ID NO: 35.

In one embodiment, the envelope protein is a fusion protein.

In one embodiment, the fusion protein is a modified fusion protein.

In one embodiment, the fusion protein is a negative sense RNA virus fusion protein, or a modified version thereof.

In one embodiment the fusion protein is a mononegavirus (i.e., of the order mononegavirales) fusion protein, or a modified version thereof.

In one embodiment, the viral fusion protein is a paramyxovirus fusion protein, or a modified version thereof.

In one embodiment, the viral fusion protein is a henipavirus fusion protein, or a modified version thereof.

In one embodiment, the viral fusion protein is a Nipah virus fusion protein (NIV-F).

In one embodiment, the viral fusion protein is a modified Nipah virus fusion protein.

In one embodiment, the Nipah virus fusion protein is a Δ22 mutant of the Nipah virus fusion protein.

In one embodiment, the viral fusion protein comprises a polypeptide tag, such as an AU1 tag. The proteins Δ22 and Δ22a are distinguished by the presence of n AU1 tag, as indicated by ‘a’.

In one embodiment, the Nipah virus fusion protein is a Δ22a mutant of the Nipah virus fusion protein.

In another embodiment, the Nipah virus fusion protein comprises a sequence according to SEQ ID NO: 3 or SEQ ID NO: 4.

In another embodiment, the Nipah virus fusion protein consists of a sequence according to SEQ ID NO: 3 or SEQ ID NO: 4.

NIV-F Δ22 (SEQ ID NO: 3):
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
ENNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSLISMLSMIILYVL
SIASLCIGLITFISFIIVEKKRNT
NIV-F Δ22a (SEQ ID NO: 4):
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSLISMLSMIILYVL
SIASLCIGLITFISFIIVEKKRNTDTYRYI

In one embodiment, the Nipah virus fusion protein comprises a sequence with at least 75% sequence identity to SEQ ID NO: 3 or 4, or a fragment thereof.

In one embodiment, the Nipah virus fusion protein comprises a sequence with at least 80% sequence identity to SEQ ID NO: 3 or 4, or a fragment thereof.

In one embodiment, the Nipah virus fusion protein comprises a sequence with at least 85% sequence identity to SEQ ID NO: 3 or 4, or a fragment thereof.

In one embodiment, the Nipah virus fusion protein comprises a sequence with at least 90% sequence identity to SEQ ID NO: 3 or 4, or a fragment thereof.

In one embodiment, the Nipah virus fusion protein comprises a sequence with at least 95% sequence identity to SEQ ID NO: 3 or 4, or a fragment thereof.

In one embodiment, the Nipah virus fusion protein comprises a sequence with at least 99% sequence identity to SEQ ID NO: 3 or 4, or a fragment thereof.

In one embodiment, the fusion protein is a rhabdovirus fusion protein, or a modified version thereof.

In one embodiment, the fusion protein is a vesicular stomatitis virus (VSV) fusion protein (VSV-G), or a modified version thereof.

In another embodiment, the fusion protein comprises a sequence according to SEQ ID NO: 34 or SEQ ID NO: 35.

In another embodiment, the fusion protein consists of a sequence according to SEQ ID NO: 34 or SEQ ID NO: 35.

In one embodiment, the viral fusion protein:
(a) is a K47Q mutant of VSV-G;
(b) is a R354A mutant of VSV-G; and/or
(c) comprises a sequence of SEQ ID NO: 34 or SEQ ID NO: 35.

VSV-G K47Q (SEQ ID NO: 34):
MKCLLYLAFLFIGVNCKFTIVFPHNQKGNWKNVPSNYHYCPSSSDLNWHN
DLIGTALQVKMPQSHKAIQADGWMCHASKWVTTCDFRWYGPKYITHSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKGLCDSNLISM
DITFFSEDGELSSLGKEGTGFRSNYFAYETGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTTERELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK
VSV-G R354A (SEQ ID NO: 35):
MKCLLYLAFLFIGVNCKFTIVFPHNQKGNWKNVPSNYHYCPSSSDLNWHN
DLIGTALQVKMPKSHKAIQADGWMCHASKWVTTCDFRWYGPKYITHSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKGLCDSNLISM
DITFFSEDGELSSLGKEGTGFRSNYFAYETGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTTEAELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK

In one embodiment, the fusion protein comprises a sequence with at least 75% sequence identity to SEQ ID NO: 34 or SEQ ID NO: 35, or a fragment thereof.

In one embodiment, fusion protein comprises a sequence with at least 80% sequence identity to SEQ ID NO: 34 or SEQ ID NO: 35, or a fragment thereof.

In one embodiment, the fusion protein comprises a sequence with at least 85% sequence identity to SEQ ID NO: 34 or SEQ ID NO: 35, or a fragment thereof.

In one embodiment, the fusion protein comprises a sequence with at least 90% sequence identity to SEQ ID NO: 34 or SEQ ID NO: 35, or a fragment thereof.

In one embodiment, the fusion protein comprises a sequence with at least 95% sequence identity to SEQ ID NO: 34 or SEQ ID NO: 35, or a fragment thereof.

In one embodiment, the fusion protein comprises a sequence with at least 99% sequence identity to SEQ ID NO: 34 or SEQ ID NO: 35, or a fragment thereof.

### Retargeting and Targeting Moieties

Viral envelope proteins may be selected based on any of a number of factors, e.g., receptor specificity, expression levels, compatibility with the vector virus, fusogenicity, availability etc. Whilst some envelope proteins may have certain preferable characteristics, they may have less favourable characteristics in another aspect.

It may be beneficial to employ retargeted viral attachment proteins, which have specificity for desired target proteins, and therefore desired cell types. Such retargeting allows improved viral vector targeting, increasing specificity and reduces unwanted off-target effects, which may have negative safety implications.

Retargeted viral attachment proteins are modified, e.g., by mutation or the addition of a retargeting moiety, to engender specificity for a target protein other than the native or endogenous receptor for which the attachment protein has specificity. The terms targeting moiety and retargeting moiety are to be understood as having the same meaning herein.

In one embodiment, the retargeted attachment protein comprises a retargeting moiety.

The choice of ‘target’, i.e., the molecule bound by the retargeting moiety, may be based on the cell type that is intended for transduction with the viral vector of the invention. For example, a molecule with a narrow cellular distribution would be a desirable target for targeting said cell type, as it would reduce off target binding and transduction, thus improving safety and specificity.

Cells may be characterised by the expression of certain surface proteins or other molecules (e.g., glycans). Specific desirable cell types can be targeted based on their expression of certain molecules. Cell surface markers are known for numerous desirable cell types, including but not limited to: lymphocytes such as T-lymphocytes and NK cells, macrophages, and hepatocytes.

By way of non-limiting example, CD8+ T lymphocytes are characterised by the expression of CD8, thus in an embodiment wherein CD8+ T lymphocytes constitute a desirable target cell, a retargeting moiety with affinity for CD8 could be used according to the invention.

By way of non-limiting example, CD3+ T lymphocytes are characterised by the expression of CD3, thus in an embodiment wherein CD3+ T lymphocytes constitute a desirable target cell, a retargeting moiety with affinity for CD3 could be used according to the invention.

In one embodiment the target molecule is a cell-surface displayed protein or molecule (e.g., glycan).

In another embodiment, the retargeting moiety has affinity for the target molecule.

By has “affinity for” it will be understood that the retargeting moiety has a suitably high affinity for its target such that binding is detectable, e.g., by methods common in the art such as SPR or flow cytometry. The affinity or avidity of the retargeting moiety is preferably sufficient to allow sufficient target binding such that viral fusion can occur and the vector can enter the target cell.

In one embodiment, the retargeting moiety is a protein.

A retargeting moiety may be bound to the attachment protein. Binding such retargeting moieties may be by a non-covalent or covalent bonds.

In one embodiment, the retargeting moiety is covalently bound to the attachment protein.

A retargeting moiety may be bound to an attachment protein at various times in the lifecycle of the protein. For example, the retargeting moiety may be covalently bound by virtue of being a chimeric protein in which the retargeting moiety and attachment proteins are encoded by a single nucleotide sequence. Alternatively, a retargeting moiety may be bound to an attachment protein after the proteins have independently folded, e.g., if co-expressed or if incubated together following expression in independent systems.

In one embodiment, the retargeted attachment protein comprises a chimeric protein.

The retargeting moiety and attachment protein may be separated by a spacer or linker. A spacer or linker may be a series of nucleotides (or polypeptides) that belong to neither of the domains that they connect and may serve to physically separate the domains or functional polypeptides that they separate. In one embodiment the retargeted attachment protein comprises a spacer or linker.

Any suitable molecule with affinity for a desired protein/receptor may be selected for use as a retargeting moiety according to the second aspect of the invention, or indeed as a target binding molecule according to the first aspect. Particularly favoured proteins are immunoglobulins, or immunoglobulin derived molecules such as: antibodies, Fabs, scFVs, nanobodies; engineered antibody mimetics such as DARPins; or molecules from known receptor-ligand axes, such as PDL1 to target PD1, and loaded (e.g., peptide loaded) MHC complexes to target T-cells. Such molecules are readily available, well studied, and have a wide variety of target specificities to choose from. Further, such molecules are known to have narrow target specificities, which allows for precision targeting. Other molecules having any or all of the foregoing features may also be suitable for use as retargeting moieties.

In one embodiment, the retargeting moiety is selected from the group consisting of an immunoglobulin, immunoglobulin derived molecule, antibody mimetic, or a molecule comprising a component of a known receptor-ligand axis.

In one embodiment, the retargeting moiety is selected from the group consisting of an antibody, a nanobody, an scFV, a DARPin, PDL1, an MHC, a loaded-MHC.

In one embodiment, the retargeting moiety is a scFV.

In one embodiment, the scFv has affinity for CD8 or CD3.

In one embodiment, the scFv has affinity for CD8.

In one embodiment, the scFv has affinity for CD3.

In one embodiment, the retargeting moiety comprises a sequence of SEQ ID NO: 5, or a fragment thereof.

In one embodiment, the retargeting moiety consists of a sequence of SEQ ID NO: 5, or a fragment thereof.

In one embodiment, the retargeting moiety comprises a sequence with at least 75% sequence identity to SEQ ID NO: 5, or a fragment thereof.

In one embodiment, the retargeting moiety comprises a sequence with at least 80% sequence identity to SEQ ID NO: 5, or a fragment thereof.

In one embodiment, the retargeting moiety comprises a sequence with at least 85% sequence identity to SEQ ID NO: 5, or a fragment thereof.

In one embodiment, the retargeting moiety comprises a sequence with at least 90% sequence identity to SEQ ID NO: 5, or a fragment thereof.

In one embodiment, the retargeting moiety comprises a sequence with at least 95% sequence identity to SEQ ID NO: 5, or a fragment thereof.

In one embodiment, the retargeting moiety comprises a sequence with at least 99% sequence identity to SEQ ID NO: 5, or a fragment thereof.

CD8 scFV (SEQ ID NO: 5):

QVQLVQSGAEDKKPGASVKVSCKASGFNIKDTYIHWVRQAPGQGLEWMGRIDPANDNTLYASKFQGRV TITADTSSNTAYMELSSLRSEDTAVYYCGRGYGYYVFDHWGQGTTVTVSSGGGGSGGGGSGGGGSDIV MTQSPSSLSASVGDRVTITCRTSRSISQYLAWYQEKPGKAPKLLIYSGSTLQSGVPSRFSGSGSGTDF TLTISSLQPEDFATYYCQQHNENPLTFGQGTKVEIK

In one embodiment, the retargeting moiety is a DARPin.

In one embodiment, the DARPin has affinity for CD8 or CD3.

In one embodiment, the DARPin has affinity for CD8.

In one embodiment, the DARPin has affinity for CD3.

In one embodiment, the retargeting moiety comprises a sequence of SEQ ID NO: 6, or a fragment thereof.

In one embodiment, the retargeting moiety consists of a sequence of SEQ ID NO: 6, or a fragment thereof.

In one embodiment, the retargeting moiety comprises a sequence with at least 75% sequence identity to SEQ ID NO: 6, or a fragment thereof.

In one embodiment, the retargeting moiety comprises a sequence with at least 80% sequence identity to SEQ ID NO: 6, or a fragment thereof.

In one embodiment, the retargeting moiety comprises a sequence with at least 85% sequence identity to SEQ ID NO: 6, or a fragment thereof.

In one embodiment, the retargeting moiety comprises a sequence with at least 90% sequence identity to SEQ ID NO: 6, or a fragment thereof.

In one embodiment, the retargeting moiety comprises a sequence with at least 95% sequence identity to SEQ ID NO: 6, or a fragment thereof.

In one embodiment, the retargeting moiety comprises a sequence with at least 99% sequence identity to SEQ ID NO: 6, or a fragment thereof.

CD8 DARPin (SEQ ID NO: 6):

DLGKKLLEASRAGQDDEVRILMANGADVNAQDRYGTTPLHLAAWHGHLEIVEVLLKHGADVNANDVKG NTPLHLAANVGHLEIVEVLLKYGADVNAADNWGHTPLHLAAFWGHLEIVEVLLKYGADVNAQDKFGKT PFDLAIDNGNEDIAEVLQKAA

In one embodiment, the retargeted attachment protein comprises the sequence of SEQ ID NO: 7 or SEQ ID NO: 36, or a fragment thereof.

In one embodiment, the retargeted attachment protein consists of the sequence of SEQ ID NO: 7 or SEQ ID NO: 36, or a fragment thereof.

In one embodiment, the retargeted attachment protein comprises a sequence with at least 75% sequence identity to SEQ ID NO: 7 or SEQ ID NO: 36, or a fragment thereof.

In one embodiment, the retargeted attachment protein comprises a sequence with at least 80% sequence identity to SEQ ID NO: 7 or SEQ ID NO: 36, or a fragment thereof.

In one embodiment, the retargeted attachment protein comprises a sequence with at least 85% sequence identity to SEQ ID NO: 7 or SEQ ID NO: 36, or a fragment thereof.

In one embodiment, the retargeted attachment protein comprises a sequence with at least 90% sequence identity to SEQ ID NO: 7 or SEQ ID NO: 36, or a fragment thereof.

In one embodiment, the retargeted attachment protein comprises a sequence with at least 95% sequence identity to SEQ ID NO: 7 or SEQ ID NO: 36, or a fragment thereof.

In one embodiment, the retargeted attachment protein comprises a sequence with at least 99% sequence identity to SEQ ID NO: 7 or SEQ ID NO: 36, or a fragment thereof.

NiV GmΔ34 CD8 DARPin (SEQ ID NO: 7):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPFREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRFNTCPAICAEGVYNDAFLIDRINWISAGVFLDSNATAA
NPVFTVFKDNEILYRAQLASEDTNAQKTITNCFLLKNKIWCISLVEIYDT
GDNVIRPKLFAVKIPEQCTGGGGSGGGGSGGGGSDLGKKLLEASRAGQDD
EVRILMANGADVNAQDRYGTTPLHLAAWHGHLEIVEVLLKHGADVNANDV
KGNTPLHLAANVGHLEIVEVLLKYGADVNAADNWGHTPLHLAAFWGHLEI
VEVLLKYGADVNAQDKFGKTPFDLAIDNGNEDIAEVLQKAA
Niv GmΔ34h CD8 DARPin (SEQ ID NO: 36):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPEREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRFNTCPAICAEGVYNDAFLIDRINWISAGVFLDSNATAA
NPVFTVFKDNEILYRAQLASEDTNAQKTITNCFLLKNKIWCISLVEIYDT
GDNVIRPKLFAVKIPEQCTGGGGSGGGGSGGGGSDLGKKLLEASRAGQDD
EVRILMANGADVNAQDRYGTTPLHLAAWHGHLEIVEVLLKHGADVNANDV
KGNTPLHLAANVGHLEIVEVLLKYGADVNAADNWGHTPLHLAAFWGHLEI
VEVLLKYGADVNAQDKFGKTPFDLAIDNGNEDIAEVLQKAAGGGSGHHHH
HH

In one embodiment, the retargeted attachment protein comprises the sequence of SEQ ID NO: 8, or SEQ ID NO: 37, or a fragment thereof.

In one embodiment, the retargeted attachment protein consists of the sequence of SEQ ID NO: 8, or SEQ ID NO: 37, or a fragment thereof.

In one embodiment, the retargeted attachment protein comprises a sequence with at least 75% sequence identity to SEQ ID NO: 8 or SEQ ID NO: 37, or a fragment thereof.

In one embodiment, the retargeted attachment protein comprises a sequence with at least 80% sequence identity to SEQ ID NO: 8 or SEQ ID NO: 37, or a fragment thereof.

In one embodiment, the retargeted attachment protein comprises a sequence with at least 85% sequence identity to SEQ ID NO: 8 or SEQ ID NO: 37, or a fragment thereof.

In one embodiment, the retargeted attachment protein comprises a sequence with at least 90% sequence identity to SEQ ID NO: 8 or SEQ ID NO: 37, or a fragment thereof.

In one embodiment, the retargeted attachment protein comprises a sequence with at least 95% sequence identity to SEQ ID NO: 8 or SEQ ID NO: 37, or a fragment thereof.

In one embodiment, the retargeted attachment protein comprises a sequence with at least 99% sequence identity to SEQ ID NO: 8 or SEQ ID NO: 37, or a fragment thereof.

NiV GmΔ34 CD8 scFv (SEQ ID NO: 8):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPFREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRFNTCPAICAEGVYNDAFLIDRINWISAGVFLDSNATAA
NPVFTVFKDNEILYRAQLASEDTNAQKTITNCFLLKNKIWCISLVEIYDT
GDNVIRPKLFAVKIPEQCTGGGGSGGGGSGGGGSAAQPAQVQLVQSGAED
KKPGASVKVSCKASGFNIKDTYIHWVRQAPGQGLEWMGRIDPANDNTLYA
SKFQGRVTITADTSSNTAYMELSSLRSEDTAVYYCGRGYGYYVFDHWGQG
TTVTVSSGGGGSGGGGSGGGGSDIVMTQSPSSLSASVGDRVTITCRTSRS
ISQYLAWYQEKPGKAPKLLIYSGSTLQSGVPSRFSGSGSGTDFTLTISSL
QPEDFATYYCQQHNENPLTFGQGTKVEIK
NiV GmΔ34h CD8 scFv (SEQ ID NO: 37):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPFREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRFNTCPAICAEGVYNDAFLIDRINWISAGVFLDSNATAA
NPVFTVFKDNEILYRAQLASEDTNAQKTITNCFLLKNKIWCISLVEIYDT
GDNVIRPKLFAVKIPEQCTGGGGSGGGGSGGGGSAAQPAQVQLVQSGAED
KKPGASVKVSCKASGFNIKDTYIHWVRQAPGQGLEWMGRIDPANDNTLYA
SKFQGRVTITADTSSNTAYMELSSLRSEDTAVYYCGRGYGYYVFDHWGQG
TTVTVSSGGGGSGGGGSGGGGSDIVMTQSPSSLSASVGDRVTITCRTSRS
ISQYLAWYQEKPGKAPKLLIYSGSTLQSGVPSRFSGSGSGTDFTLTISSL
QPEDFATYYCQQHNENPLTFGQGTKVEIKRAAARGSHHHHHH

In one embodiment, the retargeting moiety comprises a sequence of SEQ ID NO: 38, or a fragment thereof.

In one embodiment, the retargeting moiety consists of a sequence of SEQ ID NO: 38, or a fragment thereof.

In one embodiment, the retargeting moiety comprises a sequence with at least 75% sequence identity to SEQ ID NO: 38, or a fragment thereof.

In one embodiment, the retargeting moiety comprises a sequence with at least 80% sequence identity to SEQ ID NO: 38, or a fragment thereof.

In one embodiment, the retargeting moiety comprises a sequence with at least 85% sequence identity to SEQ ID NO: 38, or a fragment thereof.

In one embodiment, the retargeting moiety comprises a sequence with at least 90% sequence identity to SEQ ID NO: 38, or a fragment thereof.

In one embodiment, the retargeting moiety comprises a sequence with at least 95% sequence identity to SEQ ID NO: 38, or a fragment thereof.

In one embodiment, the retargeting moiety comprises a sequence with at least 99% sequence identity to SEQ ID NO: 38, or a fragment thereof.

CD3 DARPin (SEQ ID NO: 38):

DLGQKLLEAAWAGQLDEVRILLKAGADVNAKNSRGWTPLHTAAQTGHLEIFEVLLKAGADVNAKTNKR VTPLHLAAALGHLEIVEVLLKAGADVNARDTWGTTPADLAAKYGHRDIAEVLQKAA

In one embodiment, the retargeted attachment protein comprises the sequence of SEQ ID NO: 39, or SEQ ID NO: 40, or a fragment thereof.

In one embodiment, the retargeted attachment protein consists of the sequence of SEQ ID NO: 39, or SEQ ID NO: 40, or a fragment thereof.

In one embodiment, the retargeted attachment protein comprises a sequence with at least 75% sequence identity to SEQ ID NO: 39 or SEQ ID NO: 40, or a fragment thereof.

In one embodiment, the retargeted attachment protein comprises a sequence with at least 80% sequence identity to SEQ ID NO: 39 or SEQ ID NO: 40, or a fragment thereof.

In one embodiment, the retargeted attachment protein comprises a sequence with at least 85% sequence identity to SEQ ID NO: 39 or SEQ ID NO: 40, or a fragment thereof.

In one embodiment, the retargeted attachment protein comprises a sequence with at least 90% sequence identity to SEQ ID NO: 39 or SEQ ID NO: 40, or a fragment thereof.

In one embodiment, the retargeted attachment protein comprises a sequence with at least 95% sequence identity to SEQ ID NO: 39 or SEQ ID NO: 40, or a fragment thereof.

In one embodiment, the retargeted attachment protein comprises a sequence with at least 99% sequence identity to SEQ ID NO: 39 or SEQ ID NO: 40, or a fragment thereof.

NiV GmΔ34 CD3 DARPin (SEQ ID NO: 39):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPFREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRFNTCPAICAEGVYNDAFLIDRINWISAGVFLDSNATAA
NPVFTVFKDNEILYRAQLASEDTNAQKTITNCFLLKNKIWCISLVEIYDT
GDNVIRPKLFAVKIPEQCTGGGGSGGGGSGGGGSDLGQKLLEAAWAGQLD
EVRILLKAGADVNAKNSRGWTPLHTAAQTGHLEIFEVLLKAGADVNAKTN
KRVTPLHLAAALGHLEIVEVLLKAGADVNARDTWGTTPADLAAKYGHRDI
AEVLQKAA
NiV GmΔ34h CD3 DARPin (SEQ ID NO: 40):
MKKINEGLLDSKILSAFNTVIALLGSIVIIVMNIMIIQNYTRSTDNQAVI
KDALQGIQQQIKGLADKIGTEIGPKVSLIDTSSTITIPANIGLLGSKISQ
STASINENVNEKCKFTLPPLKIHECNISCPNPLPFREYRPQTEGVSNLVG
LPNNICLQKTSNQILKPKLISYTLPVVGQSGTCITDPLLAMDEGYFAYSH
LERIGSCSRGVSKQRIIGVGEVLDRGDEVPSLFMTNVWTPPNPNTVYHCS
AVYNNEFYYVLCAVSTVGDPILNSTYWSGSLMMTRLAVKPKSNGGGYNQH
QLALRSIEKGRYDKVMPYGPSGIKQGDTLYFPAVGFLVRTEFKYNDSNCP
ITKCQYSKPENCRLSMGIRPNSHYILRSGLLKYNLSDGENPKVVFIEISD
QRLSIGSPSKIYDSLGQPVFYQASFSWDTMIKFGDVLTVNPLVVNWRNNT
VISRPGQSQCPRFNTCPAICAEGVYNDAFLIDRINWISAGVFLDSNATAA
NPVFTVFKDNEILYRAQLASEDTNAQKTITNCFLLKNKIWCISLVEIYDT
GDNVIRPKLFAVKIPEQCTGGGGSGGGGSGGGGSDLGQKLLEAAWAGQLD
EVRILLKAGADVNAKNSRGWTPLHTAAQTGHLEIFEVLLKAGADVNAKTN
KRVTPLHLAAALGHLEIVEVLLKAGADVNARDTWGTTPADLAAKYGHRDI
AEVLQKAAGGGSGHHHHHH

Attachment proteins according to the invention may be modified, such as chemically modified. Thus, in one embodiment, the attachment protein is chemically modified.

Chemical modifications may be such that they constitute the addition, such as by covalent or noncovalent binding, of non-protein molecules to the attachment protein. Suitable non-protein molecules may be molecules that comprise part of a ligand-receptor binding axis and may be e.g., sugars, vitamins, DNA or RNA, or other synthetic polymers or small molecules. Suitably, a non-protein retargeting moiety will bind or be bound by a suitable target molecule and thus enable retargeting of the vector in which it is located.

### Viral Vector Production Systems and Cells

In a first aspect, the invention provides a viral vector production system comprising a set of nucleotide sequences, wherein the nucleotide sequences encode heterologous envelope proteins comprising at least one target binding protein and at least one viral envelope protein.

In a second aspect, the invention provides a viral vector production system comprising a set of nucleotide sequences that encode heterologous envelope protein sequences comprising at least one viral attachment protein, wherein nucleotide sequences separately encode:
(i) a non-retargeted viral attachment protein; and
(ii) a retargeted viral attachment protein.

The viral vector production system according to the second aspect of the invention facilitates production of viral vectors that comprise the ‘mixed envelopes’ as described herein.

According to the second aspect of the invention provided herein is a viral vector production system comprising a set of nucleotide sequences for the production of a viral vector with a ‘mixed envelope’.

In one embodiment, the invention provides a viral vector production system comprising a set of nucleotide sequences, wherein the nucleotide sequences comprise nucleotide sequences encoding vector components including gag-pol, env (i.e., pseudotyped envelope proteins), and optionally rev.

In a further aspect, the invention provides a cell comprising the viral vector production system of the invention.

In a further aspect, the invention provides a cell for producing viral vectors comprising the viral vector production system of the invention.

In a further aspect, the invention provides a method for producing a viral vector, comprising the steps of:
(a) introducing the viral vector production system according of the invention into a cell;
(b) optionally, selecting for a cell which comprises the nucleotide sequences of the viral vector production system according to the invention; and
(c) culturing the cell under conditions suitable for the production of the viral vector.

In one embodiment, the invention provides a method for producing a lentiviral vector, comprising the steps of:
(a) introducing the viral vector production system according of the invention into a cell;
(b) optionally, selecting for a cell which comprises the nucleotide sequences of the viral vector production system according to the invention; and
(c) culturing the cell under conditions suitable for the production of the lentiviral vector.

In a further aspect, the invention provides the use of the viral vector production system of the invention, or the cell of the invention, for producing a viral vector.

In one embodiment, the invention provides the use of the viral vector production system of the invention, or the cell of the invention, for producing a lentiviral vector.

In a further aspect, the invention provides a viral vector produced by the method or use of the invention.

In one embodiment, the invention provides a lentiviral vector produced by the method or use of the invention.

In one embodiment, there is provided a viral vector produced by the method or use according
(i) a viral fusion protein;
(ii) a non-retargeted attachment protein; and
(iii) a retargeted attachment protein.

In one embodiment, the viral vector produced by the method or use according to the invention comprises a ratio of non-retargeted attachment protein sequences or envelope displayed proteins to retargeted attachment protein sequences or envelope displayed proteins that is within the range of 0:1 to 1:0, but is neither 1:0 or 0:1, such as 0:1, 0.125:0.875, 0.25:0.75, 0.375:0.625, 0.5:0.5, 0.75:0.25, or 1:0.

In one embodiment, the invention provides a lentiviral vector production system comprises a set of nucleotide sequences encoding the components required for production of the lentiviral vector. Accordingly, a vector production system comprises a set of nucleotide sequences which encode the viral vector components necessary to generate lentiviral vector particles.

“Viral vector production system” or “vector production system” or “production system” is to be understood as a system comprising the necessary components for viral vector production, including lentiviral vector production.

In one embodiment, the lentiviral vector is derived from HIV-1, HIV-2, SIV, FIV, BIV, EIAV, CAEV or Visna lentivirus.

A “viral vector production cell”, “vector production cell”, or “production cell” is to be understood as a cell that is capable of producing a lentiviral vector or lentiviral vector particle. Lentiviral vector production cells may be “producer cells” or “packaging cells”. One or more DNA constructs of the viral vector production system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

As used herein, the term “packaging cell” refers to a cell which contains the elements necessary for production of lentiviral vector particles but which lacks the vector genome. Optionally, such packaging cells contain one or more expression cassettes which are capable of expressing viral structural proteins (such as gag, gag/pol and env).

Producer cells/packaging cells can be of any suitable cell type. Producer cells are generally mammalian cells but can be derived from other organisms, e.g. insect cells.

As used herein, the term “producer cell” or “vector producing/producer cell” refers to a cell which contains all the elements necessary for production of lentiviral vector particles. The producer cell may be either a stable producer cell line or derived transiently or may be a stable packaging cell wherein the lentiviral genome is transiently expressed.

In the methods of the invention, the vector components may include gag, env, rev and/or the RNA genome of the lentiviral vector when the viral vector is a lentiviral vector. The nucleotide sequences encoding vector components may be introduced into the cell either simultaneously or sequentially in any order.

The vector production cells may be cells cultured in vitro such as a tissue culture cell line. In some aspects of the methods and uses of the invention, suitable production cells or cells for producing a lentiviral vector are those cells which are capable of producing viral vectors or viral vector particles when cultured under appropriate conditions. Thus, the cells typically comprise nucleotide sequences encoding vector components, which may include gag, env, rev and the RNA genome of the lentiviral vector. Suitable cell lines include, but are not limited to, mammalian cells such as murine fibroblast derived cell lines or human cell lines. They are generally mammalian, including human cells, for example HEK293T, HEK293, CAP, CAP-T or CHO cells, but can be, for example, insect cells such as SF9 cells. Preferably, the vector production cells are derived from a human cell line. Accordingly, such suitable production cells may be employed in any of the methods or uses of the present invention.

Methods for introducing nucleotide sequences into cells are well known in the art and have been described previously. Thus, the introduction into a cell of nucleotide sequences encoding vector components including gag, env, rev and the RNA genome of the lentiviral vector, using conventional techniques in molecular and cell biology is within the capabilities of a person skilled in the art.

Stable production cells may be packaging or producer cells. To generate producer cells from packaging cells the vector genome DNA construct may be introduced stably or transiently. Packaging/producer cells can be generated by transducing a suitable cell line with a retroviral vector which expresses one of the components of the vector, i.e. a genome, the gag-pol components and an envelope as described in WO 2004/022761.

Alternatively, the nucleotide sequence can be transfected into cells and then integration into the production cell genome occurs infrequently and randomly. The transfection methods may be performed using methods well known in the art. For example, a stable transfection process may employ constructs which have been engineered to aid concatemerisation. In another example, the transfection process may be performed using calcium phosphate or commercially available formulations such as Lipofectamine™ 2000CD (Invitrogen, CA), FuGENE® HD or polyethylenimine (PEI). Alternatively nucleotide sequences may be introduced into the production cell via electroporation. The skilled person will be aware of methods to encourage integration of the nucleotide sequences into production cells. For example, linearising a nucleic acid construct can help if it is naturally circular. Less random integration methodologies may involve the nucleic acid construct comprising of areas of shared homology with the endogenous chromosomes of the mammalian host cell to guide integration to a selected site within the endogenous genome. Furthermore, if recombination sites are present on the construct then these can be used for targeted recombination. For example, the nucleic acid construct may contain a loxP site which allows for targeted integration when combined with Cre recombinase (i.e. using the Crellox system derived from P1 bacteriophage). Alternatively or additionally, the recombination site is an att site (e.g. from λ phage), wherein the att site permits site-directed integration in the presence of a lambda integrase. This would allow the lentiviral genes to be targeted to a locus within the host cellular genome which allows for high and/or stable expression.

Other methods of targeted integration are well known in the art. For example, methods of inducing targeted cleavage of genomic DNA can be used to encourage targeted recombination at a selected chromosomal locus. These methods often involve the use of methods or systems to induce a double strand break (DSB) e.g. a nick in the endogenous genome to induce repair of the break by physiological mechanisms such as non-homologous end joining (NHEJ). Cleavage can occur through the use of specific nucleases such as engineered zinc finger nucleases (ZFN), transcription-activator like effector nucleases (TALENs), using CRISPR/Cas9 systems with an engineered crRNA/tracr RNA (‘single guide RNA’) to guide specific cleavage, and/or using nucleases based on the Argonaute system (e.g., from T. thermophilus).

Packaging/producer cell lines can be generated by integration of nucleotide sequences using methods of just lentiviral transduction or just nucleic acid transfection, or a combination of both can be used.

Methods for generating retroviral vectors from production cells and in particular the processing of retroviral vectors are described in WO 2009/153563.

The production cell may comprise the RNA-binding protein (e.g. tryptophan RNA-binding attenuation protein, TRAP) and/or the Tet Repressor (TetR) protein or alternative regulatory proteins (e.g. CymR).

Production of lentiviral vector from production cells can be via transfection methods, from production from stable cell lines which can include induction steps (e.g. doxycycline induction) or via a combination of both. The transfection methods may be performed using methods well known in the art, and examples have been described previously.

Production cells, either packaging or producer cell lines or those transiently transfected with the lentiviral vector encoding components are cultured to increase cell and virus numbers and/or virus titres. Culturing a cell is performed to enable it to metabolize, and/or grow and/or divide and/or produce viral vectors of interest according to the invention. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture flasks, tissue culture multiwell plates, dishes, roller bottles, wave bags or in bioreactors, using batch, fed-batch, continuous systems and the like. In order to achieve large scale production of viral vector through cell culture it is preferred in the art to have cells capable of growing in suspension. Suitable conditions for culturing cells are known (see e.g. Tissue Culture, Academic Press, Kruse and Paterson, editors (1973), and R.I. Freshney, Culture of animal cells: A manual of basic technique, fourth edition (Wiley-Liss Inc., 2000, ISBN 0-471-34889-9).

Preferably cells are initially ‘bulked up’ in tissue culture flasks or bioreactors and subsequently grown in multi-layered culture vessels or large bioreactors (greater than 50L) to generate the vector producing cells for use in the present invention.

Preferably cells are grown in a suspension mode to generate the vector producing cells for use in the present invention.

The following sections describe viral vector components that may be suitably combined to comprise the viral vector production system of the invention. Such components may represent components that are common in the art of viral vectors or inventive components previously discovered by the present inventors.

### Sequence-Upgraded Polyadenylation LTRs

In eukaryotes, polyadenylation is part of the maturation of mRNA for translation and involves the addition of a polyadenine (poly(A)) tail to an mRNA transcript. The poly(A) tail comprises multiple adenosine monophosphates and is important for the nuclear export, translation and stability of mRNA. The process of polyadenylation begins as the transcription of a gene terminates. A set of cellular proteins binds to the polyA sequence elements such that the 3′ segment of the transcribed pre-mRNA is first cleaved followed by synthesis of the poly(A) sitail at the 3′ end of the mRNA. In alternative polyadenylation, a poly(A) tail is added at one of several possible sites, producing multiple transcripts from a single gene.

Native retroviral vector genomes are typically flanked by 3′ and 5′ long terminal repeats (LTRs). Native retrovirus LTRs comprise a U3 region (containing the enhancer/promoter activities necessary for transcription), and an R-U5 region that comprises important cis-acting sequences regulating a number of functions, including packaging, splicing, polyadenylation and translation. Retrovirus polyadenylation (polyA) sequences required for efficient transcriptional termination also reside within native retrovirus LTRs.

The typical structure and spacing of functional elements of polyadenylation sequences for terminating pol-II transcription have been well characterized (Proudfoot (2011), Genes & Dev. 25:1770-1782), and can be simply summarized as having: [1] a core polyadenylation signal (PAS; canonical sequence AAUAAA), [2] a cleavage site typically 15-30 nucleotides downstream of the PAS (often a ‘CA’ motif), [3] a downstream GU-rich downstream enhancer (DSE), broadly within ~100 nucleotides of the PAS (typically with 20 nucleotides for strong polyadenylation sequences), and [4] an upstream enhancer (USE), broadly within ~60 nucleotides of the PAS.

Host cell and viral gene expression levels can be regulated by the presence/absence or strength of an USE and/or DSE, and so it is recognized that there is great diversity in examples of polyadenylation sequences. Very strong viral polyadenylation sequences such as Simian Virus 40 (SV40) late polyA contain all four of these elements within a sequence less than 130 nucleotides, and strong synthetic polyA sequences based on the rabbit beta-globin polyadenylation sequence that lack a USE entirely, and is less than 50 nucleotides in total have been described (Proudfoot (2011), Genes & Dev. 25:1770-1782). Nevertheless, these four common elements are widely accepted to contribute to transcription termination efficiency, and have been shown to be employed in retroviral LTRs, including HIV-1.

As summarized above, the PAS, cleavage site and DSE for HIV-1 polyadenylation are all located across the R-U5 region of the LTR, which also forms part of the broader packaging signal for assembly of genomic vRNA in to virions. Retroviruses typically do not utilize very strong polyadenylation sequences due to the need to balance transcriptional activity driven from the 5′ LTR and efficient polyadenylation at the 3′ LTR, despite the LTRs being identical in sequence. This may be partly addressed by the fact that the 5′ LTR vRNA sequence may adopt a subtly different structure compared to the 3′ LTR due to the presence of RNA immediately downstream (which would not be present downstream of the 3′ LTR due to termination), and the lack of U3-encoded RNA at the 5′ LTR, which is present in 3′ LTR transcribed RNA (Das et al. (1999), Journal of virology 73:81-91; and Klasens et al. (1999), Nucleic Acids Res. 27:446-54). Indeed, it has been shown that the HIV-1 U3 also contains polyA enhancer sequences that overlay the promoter sequences (DeZazzo et al. (1991), Mol. Cell. Biol. 11:1624-30; and Gilmartin et al. (1995) Genes Dev. 9:72-83).

The self-inactivating LTR (SIN-LTR) feature essentially introduces a deletion within the U3 region such that enhancer/promoter activity is abolished; due to the LTR copying mechanism during reverse transcription, this results in an integrated LV genome expression cassette with no or very minimal transcriptional activity at either the 5′ or 3′ LTRs (since they are identical in sequence). This means that the only transcriptionally active component of a SIN-LTR containing LV once integrated, is from the transgene cassette. U3-deleted LTRs have been shown to have less polyadenylation activity compared to wild type, non-U3 deleted LTRs (Yang et al. (2007), Retrovirology 4:4), indicating that SIN-LTRs within LVs would be limited in the same fashion.

There are several consequences of weak PAS within LVs, and in particular SIN-LTR-containing LVs, as described below:
1) Transcriptional read-through the 3′-LTR (such as a 3′ SIN-LTR) of the LV expression cassette during vector production. This will lead to an elongated vector genome RNA (VRNA) that may be too long to package or result in low steady state abundance of vRNA. The current solution to this problem is to employ a strong heterologous polyadenylation sequence (such as the late SV40 polyA) within a few hundred nucleotides downstream of the 3′ LTR as a back-up, thus reducing the size of the vRNA in the event of transcriptional read-through the 3′-LTR.
2) Transcriptional read-through of the transgene cassette in transduced cells. This will lead to an elongated 3′ UTR for the transgene mRNA, which may lead to lower steady-state levels of the transgene mRNA.
3) Transcription of cellular genes downstream of the 3′ LTR (such as a 3′ SIN-LTR) in transduced cells due to transcriptional read-through. This will result in inappropriate expression of functional RNA or translation of the (‘dark’) proteome. Typically, by ‘dark’ proteome it is meant either ‘junk’ open-reading frames (which have no function, and will likely trigger mis-folding responses) or an ORF that was no longer transcribed into RNA (due to mutation e.g. of its upstream promoter). Unwanted transcription read-through might therefore result in expression of a gain-of-function of such ‘junk’ ORFs.
4) Transcriptional read-in through the 5′ LTR (such as a 5′ SIN-LTR) from cellular genes in transduced cells. This will lead to production of RNA encoding the LV packaging signal and RRE sequences, as well as to potential interference of the internal transgene promoter. The same mechanism may result in inappropriate expression of transgene protein in transduced cells, for example in transduced cells where the transgene expression would otherwise be restricted by a tissue specific promoter.
5) Transcriptional read-in through the 5′ LTR (such as a 5′ SIN-LTR) to the major splice donor site within the LV genome in transduced cells (this might also be possible during LV production when using circular (i.e. plasmid) DNA, as transcriptional ‘read-around’ may occur). This may allow inappropriate splicing to downstream transgene RNA and/or trans-splicing to other cellular pre-mRNA transcripts.

The present inventors previously found that modified polyA sequences can be designed to reduce (e.g. greatly minimise) and/or eliminate transcriptional read-out and/or read-in through the LV LTRs. Such modified polyA sequences may be used in the present invention. In brief, to overcome the stated problems, the invention can be defined by four major facets resulting in the new modified LTRs (termed ‘sequence-upgraded polyA’ LTRs or ‘supA-LTRs’):
1. Introducing a new PAS into the SIN/U3 region such that it becomes the primary functional PAS for polyadenylation. Another way of stating this is to ‘move’ the primary functional PAS across the transcriptional start site (TSS) boundary (the TSS is also defined as the U3/R boundary). Yet another way of describing the modification is that in the modified 3′ SIN-LTR (unlike current HIV-1 based LVs) all retained R region sequence is located downstream of the primary functional PAS.
2. Encoding a minimal but ‘sufficient’ length of R region sequence between the primary functional PAS (positioned according to 1) and a polyadenylation cleavage site, such that [1] the polyadenylation activity is high (or transcriptional read-through is demonstrated to be very low), and [2] the process of first strand transfer is efficiently retained, leading to (i.e. maintaining) high titre vector production.
3. Insertion of a sequence comprising a USE within the SIN/U3 region, thus positioning a USE close to the new, primary PAS.
4. Engineering of the 5′ R region-preferably the first stem loop (i.e. the TAR loop)—to encode a cleavage region and GU-rich DSE such that the DSE will be positioned close to the new, primary PAS within both 5′ and 3′ LTRs (i.e. they will both be supA-LTRs) after transduction/integration of the LV. The new DSE sequence must be positioned within the first stem loop such that at least the same ‘minimal but sufficient length of R region sequence is retained such that first strand transfer can occur efficiently, and ideally the engineered 5’ R region is predicted to retain a stem loop structure.

### CARe

Of the nucleotide sequences of the invention a nucleotide sequence may be provided comprising a transgene expression cassette wherein the 3′ UTR of the transgene expression cassette comprises at least one cis-acting sequence selected from (a) a cis-acting Cytoplasmic Accumulation Region (CAR) sequence; and/or (b) a cis-acting ZCCHC14 protein-binding sequence

Accordingly, in some embodiments of the nucleotide sequence comprising a lentiviral vector genome expression cassette of the invention, the lentiviral vector genome expression cassette comprises a transgene expression cassette wherein the 3′ UTR of the transgene expression cassette comprises at least one cis-acting sequence selected from (a) a cis-acting Cytoplasmic Accumulation Region (CAR) sequence; and/or (b) a cis-acting ZCCHC14 protein-binding sequence.

The term “nucleotide sequence” is synonymous with the term “polynucleotide” and/or the term “nucleic acid sequence”. The “nucleotide sequence” can be a double stranded or single stranded molecule and includes genomic DNA, cDNA, synthetic DNA, RNA and a chimeric DNA/RNA molecule. Polynucleotides may be produced recombinantly, synthetically or by any means available to those of skill in the art. They may also be cloned by standard techniques.

The nucleotide sequence may comprise a transgene expression cassette. An expression cassette is a distinct component of a vector, comprising a gene (in this case a transgene) and regulatory sequence(s) to be expressed by a transfected, transduced or infected cell. As used herein, “transgene” refers to a segment of DNA or RNA that contains a gene sequence that has been isolated from one organism and is introduced into a different organism, is a non-native segment of DNA or RNA, or is a recombinant sequence that has been made using genetic engineering techniques. The terms “transgene”, “transgene construct”, “GOI” (gene of interest) and “NOI” (nucleotide of interest) are used interchangeably herein.

The transgene expression cassettes described herein are preferably lentiviral vector transgene expression cassettes. Suitable lentiviral vector transgene expression cassettes are described in more detail elsewhere herein.

The 3′ UTR of the transgene expression cassettes described herein may comprise at least one of the novel cis-acting sequences described herein. Cis-acting sequences affect the expression of genes that are encoded in the same nucleotide sequence (i.e. the one in which the cis-acting sequence is also present). In the context of viral vectors, cis-acting sequences include the typical post-transcriptional regulatory elements (PREs) such as that from the woodchuck hepatitis virus (wPRE). General examples of cis-acting sequences are provided elsewhere herein. The terms “cis-acting element” and “cis-acting sequence” are used interchangeably herein.

In one embodiment, the 3′ UTR of the transgene expression cassette described herein comprises at least one cis-acting sequence selected from (a) a cis-acting Cytoplasmic Accumulation Region (CAR) sequence; and/or (b) a cis-acting ZCCHC14 protein-binding sequence.

As used herein, a “Cytoplasmic Accumulation Region (CAR) sequence” is a nucleotide sequence that is transcribed into mRNA and increases the stability and/or export of the mRNA to the cytoplasm and accumulation of the mRNA in the cytoplasm of a cell by sequence-dependent recruitment of the mRNA export machinery. CAR sequences have been described previously, see for example Lei et al., 2013, which describes that insertion of a CAR sequence upstream (i.e. at the 5′ end) of a naturally intronless gene can promote the cytoplasmic accumulation of the mRNA transcript.

The inventors have previously found that insertion of a CAR sequence into the 3′ UTR of a transgene expression cassette enhances gene expression. Surprisingly, these CAR sequences are shown to enhance the transgene expression from transgene cassettes utilizing introns as well as from transgene cassettes that are intronless, as well as boosting expression from cassettes already containing a full length wPRE.

Suitable CAR sequences for insertion into the 3′ UTR of a transgene expression cassette may be readily identifiable by a person of skill in the art, based on their common general knowledge (see e.g. the disclosure in Lei et al., 2013, which is incorporated herein in its entirety).

The CAR sequences may comprise at least one CAR element (CARe) sequence. As would be understood by a person of skill in the art, a CARe sequence is a core sequence that is present within a CAR sequence (and typically, wherein the CARe sequence is repeated a number of times within the CAR sequence). Examples of CARe sequences are described in Lei et al., 2013.

### Interfering RNA

Introns within the transgene expression cassette (e.g. the intron within the EF1a promoter if employed as the internal transgene promoter) provide a boost to expression in certain target cells. However, such additional introns within the transgene expression cassette may be spliced-out during transcription. A way of ensuring transgene intron retention within vRNA is to invert the transgene cassette so that the intron within the transgene expression cassette is not recognised as such because it is in the anti-sense direction with respect to the retroviral vector genome expression cassette.

Accordingly, the transgene expression cassette may be inverted with respect to the retroviral vector genome expression cassette. In other words, the internal transgene promoter and transgene sequences oppose the retroviral vector genome cassette promoter such that the retroviral vector genome and transgene are in opposed transcriptional orientations. Thus, the transgene expression cassette may be in the antisense orientation (i.e. encoded on the antisense strand/the bottom strand) with respect to the retroviral vector genome expression cassette.

Alternatively, the transgene expression cassette may be in the forward orientation with respect to the retroviral vector genome expression cassette. In other words, the internal transgene promoter and transgene sequences are in the same orientation as the retroviral vector genome cassette promoter such that the retroviral vector genome and transgene are in the same transcriptional orientation. Thus, the transgene expression cassette may be in the sense orientation (i.e. encoded on the sense strand/on the top strand) with respect to the retroviral vector genome expression cassette.

Hence, the transgene expression cassette can be inverted or non-inverted (i.e. in the forward orientation) with respect to the retroviral vector genome cassette.

If a tissue specific promoter is utilized as the internal transgene promoter that is not/minimally active during retroviral vector production then the inverted transgene approach requires no further considerations. However, should the transgene promoter generate sufficient levels of transgene mRNA during retroviral vector production then the possibility of generating long dsRNA products via vRNA: mRNA annealing increases, and this will trigger innate dsRNA sensing pathways, such as those involving oligoadenylate synthetase-ribonuclease L (OAS-RNase L), protein kinase R (PKR), and interferon (IFN)/melanoma differentiation-associated protein 5 (MDA-5). Whilst a number of these pathways are likely to be (partly) defective in HEK293 (T) cells (Ferreira, C., B., et. al., Mol Ther Methods Clin Dev. (2019); 17:209-219.), the inventors have previously shown that generation of cytoplasmic dsRNA results in suppression of de novo protein synthesis.

The present inventors previously found that RNAi can be employed in retroviral vector production cells to suppress the expression of the NOI (i.e. transgene) during retroviral vector production in order to minimize unwanted effects of the transgene protein during vector production and/or to rescue titres of retroviral vectors harbouring an actively transcribed inverted transgene cassette. The inventors surprisingly found that RNAi can be employed during vector production to minimize and/or eliminate mRNA encoding the transgene but not vector genome RNA (vRNA) required for packaging. Thus, the amount of transgene mRNA produced to be available for forming dsRNA with the vRNA is reduced, minimizing the impact on titres of retroviral vectors harbouring an actively transcribed inverted transgene cassette. This feature also provides a mechanism for transgene repression during LV production, which has previously shown to be an effective way of rescuing titres for genomes encoding toxic or ‘problematic’ transgene proteins.

In other words, the use of interfering RNA(s) specific for the transgene mRNA provides a mechanism for avoiding de novo protein synthesis inhibition and/or the consequences of other dsRNA sensing pathway and enables rescue of inverted transgene retroviral vector titres. The interfering RNA is targeted to the transgene mRNA so that any mRNA that does locate to the cytoplasm is a target for RNAi-mediated degradation and/or cleavage, preferably cleavage.

Preferably, the interfering RNA(s) employed result in cleavage of mRNA encoding the transgene in order to minimize and/or eliminate the formation of dsRNA. Suitably, the interfering RNA(s) target the mRNA encoding the transgene for cleavage. The interfering RNA(s) may target the mRNA encoding the transgene for cleavage by the RNA-induced silencing complex (RISC).

As used herein, the term “is specific for” means that the interfering RNA preferentially binds to mRNA encoding the transgene over an mRNA molecule which does not encode the transgene. Thus, the interfering RNA targets the mRNA encoding the transgene.

As used herein, the term “interfering RNA” means an RNA which is capable of mediating RNA interference (RNAi).

Interfering RNAs may be, for example, a siRNA; a sisiRNA; a tsiRNA; a RNA-DNA chimeric duplex; a tkRNA; a Dicer-substrate dsRNA; a shRNA; a tRNA-shRNA; an aiRNA; a pre-miRNA; a pri-miRNA mimic; a pri-miRNA mimic cluster; and combinations thereof.

The interfering may be synthetic. Synthetic interfering RNAs are suitable for use by transient co-transfection during lentiviral vector production, i.e. may be provided in trans to the viral vector genome expression cassette in a retroviral vector production cell.

Suitably, the interfering RNA may be provided in cis to the viral vector genome expression cassette in a lentiviral vector production cell, i.e. the vector intron may comprise the interfering RNA. Examples of interfering RNAs which maybe provided in cis include a siRNA; a shRNA; a tRNA-shRNA; a pre-miRNA; a pri-miRNA mimic; a pri-miRNA mimic cluster; and combinations thereof.

Preferably, the interfering RNA is siRNA, shRNA or miRNA. Preferably, the interfering RNA is shRNA or miRNA. Preferably, the interfering RNA for use according to the invention is a miRNA.

In some embodiments, the guide strand of the interfering RNA (preferably, miRNA) is fully complementary to the target sequence of the transgene mRNA. In this instance, the guide strand is 100% matched, i.e. 100% complementary, to its target. Thus, the guide strand may target the RISC complex to the target sequence of the mRNA encoding the transgene (i.e. transgene mRNA) Watson-Crick base pairing. The vRNA is not targeted by the guide strand in order to avoid degradation and/or cleavage of vRNA required for packaging.

In one embodiment, the guide strand of the interfering RNA, preferably miRNA, does not comprise a mismatch with the target sequence within the mRNA encoding the transgene.

As used herein, the term “mismatch” refers to the presence of an uncomplimentary base. Thus, a “mismatch” refers to an uncomplimentary base in the guide strand or passenger strand which is not capable of Watson-Crick base pairing with the complementary sequence within the mRNA encoding the transgene or vRNA, respectively.

Preferably, when employing the interfering RNA to target mRNA encoding the transgene derived from an inverted transgene cassette, the interfering RNA (preferably miRNA) is designed such that the passenger strand is mismatched (i.e. not 100% complementary) to its complementary sequence in the vRNA. In this way, should the passenger strand be used as the guide strand in a RISC, it will not lead to cleavage of the vRNA, which would otherwise lead to reduction in vRNA available for packaging.

In some embodiments, the interfering RNA (preferably miRNA) comprises a passenger strand which comprises at least one mismatch (suitably, at least two, or at least three)—preferably at position 2, 9, 10, or 11 of the passenger stand-with its complimentary sequence within the RNA genome of the retroviral vector.

In one embodiment, the passenger strand of the interfering RNA (preferably miRNA) imperfectly matches its target vector genome sequence resulting in a central bulge.

In some embodiments, the set of nucleic acid sequences comprises multiple nucleic acid sequences encoding a plurality of interfering RNAs specific for multiple target nucleotide sequences. Preferably, each interfering RNA is specific for a different target nucleotide sequence.

The interfering RNA(s) can be provided in trans or in cis during lentiviral vector production. Cis elements are present on the same molecule of DNA as the gene they affect whereas trans elements can affect genes distant from the gene from which they were transcribed.

Thus, an interfering RNA expression cassette may be co-expressed with lentiviral vector components during lentiviral vector production. The interfering RNA is an interfering RNA as described herein. Thus, the interfering RNA targets the mRNA encoding the transgene, leading to mRNA degradation and concomitant reduction in dsRNA and transgene expression. Such an interfering RNA expression cassette may be easily constructed by those skilled in the art, for example driven by a U6 pol-III promoter or tRNA promoter. The target of the interfering RNA may be the sequence of the 3′ UTR of the transgene mRNA encompassed by the VI.

In some alternative embodiments, the lentiviral vector genome expression cassette further comprises a vector intron. Suitably, the vector intron comprises the nucleic acid sequence encoding the interfering RNA. Thus, the interfering RNA may be provided in cis during lentiviral vector production.

Inverted transgene expression cassettes are able to retain an intron (herein termed “vector intron” (VI)) within the transgene cassette. By contrast, in non-inverted cassettes the transgene cassette would be spliced out together with the VI, which is undesirable. Thus, the VI is provided in the forward orientation (i.e. on the sense strand) with respect to the retroviral vector genome expression cassette. This permits out-splicing of the VI during transcription of the lentiviral vector genome. The inverted transgene expression cassettes of the invention may make use of the VI to reduce transgene mRNA expression during vector production, since an interfering RNA which is specific for the mRNA encoding the transgene can be inserted within the 3′ UTR of the inverted transgene expression cassette, such that the interfering RNA is also encompassed by the VI on the sense strand. Thus, transgene mRNA is destabilized and/or degraded within vector production cells. By contrast, upon splicing-out of the VI, the interfering RNA is also removed from the packaged genome of the lentiviral vector (e.g. vRNA) such that the transgene mRNA will lack the interfering RNA in transduced cells. As such, critically, the interfering RNA are only active during lentiviral vector production because they are lost from the packaged vRNA by splicing-out of the VI.

One or more interfering RNAs can be inserted within the antisense VI sequence, i.e. the inverted VI.

By way of illustrative example, the interfering RNA may be one or more ‘self-cleaving’ miRNAs that are located within the 3′ UTR of the transgene expression cassette. The one or more miRNAs (i.e. pre-miRNAs) are therefore cleaved from the 3′ UTR of the mRNA encoding the transgene (thus removing the polyA tail of the mRNA, leading to destabilisation of the mRNA encoding the transgene), and then are processed by DROSHA/Dicer into mature miRNAs and loaded into the RISC to target sequences within the transgene mRNA such that further cleavage can occur. dsRNA fragments are loaded into RISC with each strand having a different fate based on the asymmetry rule phenomenon, the selection of one strand as the guide strand over the other based on thermodynamic stability. The newly generated miRNA or siRNA act as single-stranded guide sequences for RISC to target mRNA for degradation. The strand with the less thermodynamically stable 5′ end is selected by the protein Argonaute and integrated into RISC. This strand is known as the guide strand and targets mRNA for degradation. The other strand, known as the passenger strand, is degraded by RISC.

By ‘self-cleaving’ it is meant that, since the miRNA(s) are located within the 3′ UTR of the mRNA encoding the transgene and target the mRNA encoding the transgene, the miRNA(s) are self-targeting for cleavage.

The interfering RNA may be specific for a sequence within the 5′ UTR and/or coding-region and/or 3′UTR of the transgene expression cassette (i.e. the 5′ UTR and/or coding-region and/or 3′UTR of the transgene expression cassette comprises a target nucleotide sequence). Suitably, the target nucleotide sequence may be within the 5′ UTR of the transgene expression cassette. Alternatively, the target nucleotide sequence may be within the 3′ UTR. If a plurality of interfering RNAs are employed, the target nucleotide sequences may be within the 3′ UTR and the 5′ UTR of the transgene expression cassette.

In some embodiments, the 3′ UTR or the 5′ UTR of the transgene expression cassette comprises at least one target nucleotide sequence.

In some embodiments, the transgene expression cassette is genetically engineered to comprise at least one target nucleotide sequence within the 3′ UTR or 5′ UTR. The at least one target nucleotide sequence may be at least one predetermined heterologous target nucleotide sequence for which efficient interfering RNAs are already available.

Molecular cloning methods to introduce a nucleotide sequence into a target sequence are known in the art. For example, conventional techniques of molecular biology (described elsewhere herein) may be employed.

In some embodiments, the interfering RNA is specific for the at least one target nucleotide sequence.

As used herein, the term “target nucleotide sequence” means a sequence within the transgene expression cassette to which the interfering RNA binds. Suitably, the target nucleotide sequence is 100% complementary to the guide strand of the interfering RNA, which is preferably a miRNA.

As described herein, the interfering RNA may be provided in cis during retroviral vector production.

Accordingly, the invention provides an expression cassette encoding a lentiviral vector genome comprising:
(i) a transgene expression cassette; and
(ii) a vector intron comprising at least one interfering RNA as described herein.

In a further embodiment, the invention provides a retroviral vector genome comprising a transgene expression cassette and a vector intron, wherein the vector intron comprises at least one interfering RNA as described herein.

In some embodiments, the transgene expression cassette is inverted with respect to the retroviral vector genome expression cassette.

In one embodiment, the invention provides a nucleotide sequence comprising the expression cassette of the invention.

An illustrative example of the use of one or more interfering RNAs to target transgene mRNA for cleavage by the RISC complex in order to [1] reduce transgene expression during retroviral vector production and [2] avoid the production of dsRNA when employing transgene cassettes containing active transcription units that are inverted with respect to the vector genome cassette, and thus would otherwise lead to dsRNA sensing pathways detrimental to vector production is provided below.

### Illustrative Example

The illustrative example of a process flow of implementing this approach can be summarized as follows:

1. Identify One or More Target Sites within the Transgene mRNA Sequence to Enable Efficient mRNA Cleavage(s), Leading to Reduced Steady-State Levels of mRNA, Reduction of Transgene Protein Expression and Rescue of Titres (for Inverted Transgenes).

This can be achieved in a number of ways. Firstly, the transgene mRNA sequence can be in silico screened for potential sites that are predicted to be good targets for microRNA (such services are commercially available, for example:
iScore (http://www.med.nagoya-u.ac.jp/neurogenetics/i_Score/i_score.html);
Invitrogen (http://rnaidesigner.invitrogen.com/rnaiexpress/design.do);
Thermo Fisher (http://www.thermoscientificbio.com/design-center/);
siSPOTR (https:/sispotr.icts.uiowa.edu/sispotr/tools/sispotr).

This process involves:
I. Target sequence analysis
II. Thermodynamic profiling of guide strand
III. Addition of passenger strand mis-matches if required
IV. Assessment of microRNA secondary structure (e.g. via m-fold)
V. Selected siRNA candidates tested for off target effects by blasting (e.g. NCBI BLAST) against human transcriptome using the following settings: word size-7; threshold 1000; mismatch penalty-1 or 0; gap open 3; extension 2. (Such loose criteria ensure an effective search for siRNA off-targets, unlike using the default BLAST settings.)

Several (for example, up to 10) siRNAs or shRNAs can be generated and screened against a simple transgene expression cassette within the production cells, thus identifying one or more siRNA/shRNAs that can be used in the invention.

Alternatively, a predetermined heterologous target sequence for which there is already available efficient siRNA/shRNAs can be cloned within the 5′ or 3′ UTRs of the transgene cassette and empirically tested for mRNA cleavage and transgene protein knock-down by supplying these siRNA/shRNAs in co-transfection experiments.

Alternatively, the production cell may be characterized for endogenous microRNAs that are highly expressed constitutively, and/or under vector production conditions (for example microRNAs upregulated by sodium butyrate induction). The target sequence(s) of these endogenous microRNAs can be cloned into the 5′ or 3′ UTRs of the transgene cassette and empirically tested for mRNA cleavage and transgene protein knock-down.

Multiple target sites of one or more microRNAs can optionally be cloned into 5′ and/or 3′ UTRs of the transgene cassette.

It is desirable for the guide strand of the microRNA to be designed such that the resultant active RISC mediates transgene mRNA cleavage i.e. the guide strand is fully complementary to the target sequence. Optionally, microRNAs can be designed for use with inverted transgene cassettes such that the passenger strand is mis-matched in order to minimize any possible cleavage of vRNA should the passenger strand be loaded into a RISC.

2. Choice of microRNA Modality

The siRNA/shRNA/miRNA identified to induce the desired levels of transgene repression and/or mRNA cleavage can be used directly in co-transfection production of retroviral vectors. Alternatively, the interfering RNA can be designed as part of an expression cassette in order to be de novo transcribed during vector production, for example, by a polymerase-Ill promoter such as U6 or a tRNA promoter. Thus, a plasmid encoding the miRNA cassette can be co-transfected into the production cell together with vector component plasmids. The miRNA plasmid may contain multiple single miRNA expression cassettes, or a single expression cassette encoding multiple tandem miRNAs processed from a single transcript. Alternatively, the miRNA expression cassette(s) may be stably integrated into the host cell DNA or stably maintained as an episome.

Alternatively, such miRNA expression cassette(s) may be cloned into the vector genome or packaging plasmids in cis.

### 3. Optimisation of Vector Production

Depending on the mode of microRNA being employed for the implementation of transgene mRNA knock-down during vector production, the process can be optimized to achieve the maximal effect i.e. efficient transgene repression and/or recovery in titres of vectors containing an inverted transgene cassette.

For transient production utilizing siRNA/shRNA/miRNA, this will involve empirically testing different amounts and ratios of interfering RNA effectors relative to plasmids encoding vector components and transfection reagent, as well as harvest times and/or sodium butyrate induction levels/timings.

For the approach where the miRNA cassette(s) is inserted in cis on one or more plasmids encoding vector components, the number, position and orientation of the miRNA(s) should be empirically tested.

For development of stable a cell line expressing miRNA(s), the screening process will empirically test and identify clones that have low transgene levels and high vector titres.

### Use of an Interfering RNA

The invention provides the use of a nucleic acid sequence encoding an interfering RNA as described herein for repressing expression of a transgene and/or increasing retroviral titre during retroviral vector production.

The invention provides the use of a nucleic acid sequence encoding an interfering RNA as described herein for repressing expression of a transgene and/or increasing retroviral titre in a retroviral vector production cell.

Suitably, the nucleic acid sequence encoding an interfering RNA as described herein is used in conjunction with nucleotide sequences encoding retroviral vector components. In other words, the nucleic acid sequence encoding an interfering RNA may be used as part of a set of nucleic acid sequences as described herein.

Titres of vectors containing actively expressed inverted transgene cassettes may be negatively impacted due to the triggering of innate dsRNA sensing pathways within the cell leading to loss of de novo protein synthesis as described above. Thus, titres of vectors containing actively expressed inverted transgene cassettes may be enhanced by the use of an interfering RNA as described herein to target the transgene mRNA during retroviral vector production, thereby preventing the triggering of innate dsRNA sensing pathways and the loss of de novo protein synthesis.

In addition, certain transgenes may be toxic to the cell or have other deleterious properties when expressed in a cell. Thus, the use of an interfering RNA as described herein to target the transgene mRNA during retroviral vector production may further boost titres of vectors harbouring such a transgene.

Suitably, titres of vectors containing actively expressed inverted transgene cassettes may be restored to the titre levels seen during production of a retroviral vector harbouring a reporter gene construct (e.g. a GFP transgene) by the use of an interfering RNA as described herein to target the transgene mRNA during retroviral vector production.

Accordingly, the use of an interfering RNA as described herein may enhance the titre of a retroviral vector containing an actively expressed inverted transgene cassette during retroviral vector production relative to retroviral vector production of the corresponding retroviral vector in the absence of an interfering RNA as described herein. Thus, production of a retroviral vector containing an actively expressed inverted transgene cassette in the presence of an interfering RNA as described herein enhances retroviral vector titre relative to retroviral vector production of the corresponding retroviral vector in the absence of an interfering RNA as described herein. The present invention is particularly advantageous for enhancing the titre of retroviral vectors harbouring an actively transcribed inverted transgene cassette and a transgene which is, for example, toxic to the cell.

A suitable assay for the measurement of retroviral vector titre is as described herein. Suitably, the retroviral vector production involves co-expression of said interfering RNA with vector components including gag, env, rev and the genome of the retroviral vector. Alternatively, the retroviral vector production involves provision of said interfering RNA in cis.

In some embodiments, the use of an interfering RNA as described herein may increase retroviral vector titre of a retroviral vector containing an actively expressed inverted transgene cassette during retroviral vector production by at least 30% relative to retroviral vector production of the corresponding retroviral vector in the absence of an interfering RNA as described herein. Suitably, the use of an interfering RNA as described herein may increase retroviral vector titre of a retroviral vector containing an actively expressed inverted transgene cassette during retroviral vector production by at least 35% (suitably at least 40%, 45%, 50%, 60%, 70%, 100%, 150%, 200%, 250%, 300%, 350%, 400%, 450%, 500%, 550%, 600%, 650%, 700%, 750%, 800%, 850%, 900%, 950% or 1000%) relative to retroviral vector production of the corresponding retroviral vector in the absence of an interfering RNA as described herein.

As described above, expression of the transgene protein during retroviral vector production may have unwanted effects on vector virion assembly, vector virion activity, process yields and/or final product quality. Therefore, it is desirable to repress expression of the transgene in viral vector production cells. Suitably, the translation of the mRNA encoding the transgene may be repressed.

Repression or prevention of the translation of the NOI (i.e. transgene) is to be understood as alteration of the amount of the product (e.g. transgene protein) encoded by the NOI that is translated during viral vector production in comparison to the amount translated in the absence of the interfering RNA as described herein at the equivalent time point.

In one embodiment, expression of the transgene is repressed or prevented in a retroviral vector production cell.

The expression of the protein from the transgene at any given time during vector production may be reduced to 90% (suitably, to 80%, 70%, 60%, 50%, 40%, 30%, 20%, 10%, 5%, 4%, 3%, 2%, 1%, 0.5% or 0.1%) of the amount expressed in the absence of the interfering RNA as described herein at the same time-point in vector production.

The expression of the protein from the transgene at any given time during vector production may be reduced to less than 90% (suitably, to less than 80%, 70%, 60%, 50%, 40%, 30%, 20%, 10%, 5%, 4%, 3%, 2%, 1%, 0.5% or 0.1%) of the amount expressed in the absence of the interfering RNA as described herein at the same time-point in vector production.

In one embodiment, translation of the transgene is repressed or prevented in a retroviral vector production cell.

Preventing the expression of the protein from the transgene is to be understood as reducing the amount of the protein that is expressed to substantially zero (suitably, to zero).

The translation of the transgene at any given time during vector production may be reduced to 90% (suitably, to 80%, 70%, 60%, 50%, 40%, 30%, 20%, 10%, 5%, 4%, 3%, 2%, 1%, 0.5% or 0.1%) of the amount translated in the absence of the interfering RNA as described herein at the same time-point in vector production.

The translation of the NOI at any given time during vector production may be reduced to less than 90% (suitably, to less than 80%, 70%, 60%, 50%, 40%, 30%, 20%, 10%, 5%, 4%, 3%, 2%, 1%, 0.5% or 0.1%) of the amount translated in the absence of the interfering RNA as described herein at the same time-point in vector production.

Preventing the translation of the NOI is to be understood as reducing the amount of translation to substantially zero (suitably, to zero).

Methods for the analysis and/or quantification of the translation of a transgene are well known in the art.

A protein product from lysed cells may be analysed using methods such as SDS-PAGE analysis with visualisation by Coomassie or silver staining. Alternatively a protein product may be analysed using Western blotting or enzyme-linked immunosorbent assays (ELISA) with antibody probes which bind the protein product. A protein product in intact cells may be analysed by immunofluorescence.

### RNA Splicing

The present invention, as disclosed herein, may be combined with major splice donor (MSD) site knock out lentiviral vector genomes. The invention may employ lentiviral vector genomes in which the major splice donor site, and optionally the cryptic splice donor site 3′ to the major splice donor site, are inactivated.

Thus, in some embodiments, the major splice donor site in the genome of the lentiviral vector, and optionally the cryptic splice donor site 3′ to the major splice donor site in the genome of the lentiviral vector are inactivated.

In some embodiments, the inactivated major splice donor site has the sequence set forth in SEQ ID NO: 12.

Suitable inactivated splice sites for use according to the present invention are described in WO 2021/160993 and incorporated herein by reference.

RNA splicing is catalysed by a large RNA-protein complex called the spliceosome, which is comprised of five small nuclear ribonucleoproteins (snRNPs). The borders between introns and exons are marked by specific nucleotide sequences within a pre-mRNA, which delineate where splicing will occur. Such boundaries are referred to as “splice sites”. The term “splice site” refers to polynucleotides that are capable of being recognized by the splicing machinery of a eukaryotic cell as suitable for being cut and/or ligated to another splice site.

Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site”. Splice sites include, for example, naturally occurring splice sites, engineered or synthetic splice sites, canonical or consensus splice sites, and/or non-canonical splice sites, for example, cryptic splice sites.

Splice acceptor sites generally consist of three separate sequence elements: the branch point or branch site, a polypyrimidine tract and the acceptor consensus sequence. The branch point consensus sequence in eukaryotes is YNYTRAC (where Y is a pyrimidine, N is any nucleotide, and R is a purine). The 3′ acceptor splice site consensus sequence is YAG (where Y is a pyrimidine) (see, e.g., Griffiths et al., eds., Modern Genetic Analysis, 2nd edition, W.H. Freeman and Company, New York (2002)). The 3′ splice acceptor site typically is located at the 3′ end of an intron.

The terms “canonical splice site” or “consensus splice site” may be used interchangeably and refer to splice sites that are conserved across species.

Consensus sequences for the 5′ donor splice site and the 3′ acceptor splice site used in eukaryotic RNA splicing are well known in the art. These consensus sequences include nearly invariant dinucleotides at each end of the intron: GT at the 5′ end of the intron, and AG at the 3′ end of an intron.

The canonical splice donor site consensus sequence may be (for DNA) AG/GTRAGT (where A is adenosine, T is thymine, G is guanine, C is cytosine, R is a purine and “/” indicates the cleavage site). This conforms to the more general splice donor consensus sequence MAGGURR described herein. It is well known in the art that a splice donor may deviate from this consensus, especially in viral genomes where other constraints bear on the same sequence, such as secondary structure for example within a vRNA packaging region. Non-canonical splice sites are also well known in the art, albeit they occur rarely compared to the canonical splice donor consensus sequence.

By “major splice donor site” is meant the first (dominant) splice donor site in the viral vector genome, encoded and embedded within the native viral RNA packaging sequence typically located in the 5′ region of the viral vector nucleotide sequence.

In one embodiment, the lentiviral vector genome does not contain an active major splice donor site, that is splicing does not occur from the major splice donor site, and splicing activity from the major splice donor site is ablated.

The major splice donor site is located in the 5′ packaging region of a lentiviral genome.

In the case of the HIV-1 virus, the major splice donor consensus sequence is (for DNA) TG/GTRAGT (where A is adenosine, T is thymine, G is guanine, C is cytosine, R is a purine and “/” indicates the cleavage site).

In one embodiment, the splice donor region, i.e. the region of the vector genome which comprises the major splice donor site prior to mutation, may have the following sequence:

(SEQ ID NO: 9)
GGGGCGGCGACTGGTGAGTACGCCAAAAAT

In one embodiment, the mutated splice donor region may comprise the sequence:

(SEQ ID NO: 10-MSD-2KO)
GGGGCGGCGACTGCAGACAACGCCAAAAAT

In one embodiment, the mutated splice donor region may comprise the sequence:

(SEQ ID NO: 11-MSD-2KOv2)
GGGGCGGCGAGTGGAGACTACGCCAAAAAT

In one embodiment, the mutated splice donor region may comprise the sequence:

(SEQ ID NO: 12-MSD-2KOm5)
GGGGAAGGCAACAGATAAATATGCCTTAAAAT

In one embodiment, prior to modification the splice donor region may comprise the sequence:

(SEQ ID NO: 13)
GGCGACTGGTGAGTACGCC

This sequence is also referred to herein as the “stem loop 2” region (SL2). This sequence may form a stem loop structure in the splice donor region of the vector genome. In one embodiment of the invention this sequence (SL2) may have been deleted from the nucleotide sequence according to the invention as described herein.

As such, the invention encompasses the use of a lentiviral vector genome that does not comprise SL2. The invention encompasses the use of a lentiviral vector genome that does not comprise a sequence according to SEQ ID NO: 13.

In one embodiment of the invention the major splice donor site may have the following consensus sequence, wherein R is a purine and “/” is the cleavage site:

TG/GTRAGT

In one embodiment, R may be guanine (G).

In one embodiment of the invention, the major splice donor and cryptic splice donor region may have the following core sequence, wherein “/” are the cleavage sites at the major splice donor and cryptic splice donor sites:

/GTGA/GTA.

In one embodiment of the invention the MSD-mutated vector genome may have at least two mutations in the major splice donor and cryptic splice donor ‘region’ (/GTGA/GTA), wherein the first and second ‘GT’ nucleotides are the immediately 3′ of the major splice donor and cryptic splice donor nucleotides respectively

In one embodiment of the invention the major splice donor consensus sequence is CTGGT. The major splice donor site may contain the sequence CTGGT.

In one embodiment the nucleotide sequence encoding the lentiviral vector genome, prior to inactivation of the splice sites, comprises the sequence as set forth in any of SEQ ID NOs: 9, 13 and/or the sequence TG/GTRAGT, CTGGT, TGAGT and/or/GTGA/GTA.

In one embodiment the nucleotide sequence comprises an inactivated major splice donor site which would otherwise have a cleavage site between nucleotides corresponding to nucleotides 13 and 14 of SEQ ID NO: 9.

According to the invention as described herein, the nucleotide sequence encoding the lentiviral vector genome also contains an inactive cryptic splice donor site. In one embodiment the nucleotide sequence does not contain an active cryptic splice donor site adjacent to (3′ of) the major splice donor site, that is to say that splicing does not occur from the adjacent cryptic splice donor site, and splicing from the cryptic splice donor site is ablated.

The term “cryptic splice donor site” refers to a nucleic acid sequence which does not normally function as a splice donor site or is utilised less efficiently as a splice donor site due to the adjacent sequence context (e.g. the presence of a nearby ‘preferred’ splice donor), but can be activated to become a more efficient functioning splice donor site by mutation of the adjacent sequence (e.g. mutation of the nearby ‘preferred’ splice donor).

In one embodiment the cryptic splice donor site is the first cryptic splice donor site 3′ of the major splice donor.

In one embodiment the cryptic splice donor site is within 6 nucleotides of the major splice donor site on the 3′ side of the major splice donor site. Preferably the cryptic splice donor site is within 4 or 5, preferably 4, nucleotides of the major splice donor cleavage site.

In one embodiment of the invention the cryptic splice donor site has the consensus sequence

TGAGT.

In one embodiment the inactivated cryptic splice donor site which would otherwise have a cleavage site between nucleotides corresponding to nucleotides 17 and 18 of SEQ ID NO: 9.

In one embodiment of the invention the major splice donor site and/or adjacent cryptic splice donor site contain a “GT” motif. In one embodiment of the invention both the major splice donor site and adjacent cryptic splice donor site contain a “GT” motif which is mutated. The mutated GT motifs may inactivate splice activity from both the major splice donor site and adjacent cryptic splice donor site. An example of such a mutation is referred to herein as “MSD-2KO”.

In one embodiment the splice donor region may comprise the following sequence:

CAGACA

For example, in one embodiment the mutated splice donor region may comprise the following sequence:

(SEQ ID NO: 14)
GGCGACTGCAGACAACGCC

A further example of an inactivating mutation is referred to herein as “MSD-2KOv2”.

In one embodiment the mutated splice donor region may comprise the following sequence:

GTGGAGACT

For example, in one embodiment the mutated splice donor region may comprise the following sequence:

(SEQ ID NO: 15)
GGCGAGTGGAGACTACGCC

For example, in one embodiment the mutated splice donor region may comprise the following sequence:

(SEQ ID NO: 16)
AAGGCAACAGATAAATATGCCTT

In one embodiment the stem loop 2 region as described above may be deleted from the splice donor region, resulting in inactivation of both the major splice donor site and the adjacent cryptic splice donor site. Such a deletion is referred to herein as “ASL2”.

A variety of different types of mutations can be introduced into the nucleic acid sequence in order to inactivate the major and adjacent cryptic splice donor sites.

In one embodiment the mutation is a functional mutation to ablate or suppress splicing activity in the splice region. The nucleotide sequence may contain a mutation or deletion in any of the nucleotides in any of SEQ ID NOs: 9, 13 and/or the sequence TG/GTRAGT, CTGGT, TGAGT and/or/GTGA/GTA. Suitable mutations will be known to one skilled in the art, and are described herein.

For example, a point mutation can be introduced into the nucleic acid sequence. The term “point mutation,” as used herein, refers to any change to a single nucleotide. Point mutations include, for example, deletions, transitions, and transversions; these can be classified as nonsense mutations, missense mutations, or silent mutations when present within protein coding sequence. A “nonsense” mutation produces a stop codon. A “missense” mutation produces a codon that encodes a different amino acid. A “silent” mutation produces a codon that encodes either the same amino acid or a different amino acid that does not alter the function of the protein. One or more point mutations can be introduced into the nucleic acid sequence comprising the cryptic splice donor site. For example, the nucleic acid sequence comprising the cryptic splice site can be mutated by introducing two or more point mutations therein.

At least two point mutations can be introduced in several locations within the nucleic acid sequence comprising the major splice donor and cryptic splice donor sites to achieve attenuation of splicing from the splice donor region. In one embodiment the mutations may be within the four nucleotides at the splice donor cleavage site; in the canonical splice donor consensus sequence this is A¹G²/G³T⁴, wherein “/” is the cleavage site. It is well known in the art that a splice donor cleavage site may deviate from this consensus, especially in viral genomes where other constraints bear on the same sequence, such as secondary structure for example within a vRNA packaging region. It is well known that the G³T⁴ dinucleotide is generally the least variable sequence within the canonical splice donor consensus sequence, and mutations to the G³ and or T⁴ will most likely achieve the greatest attenuating effect. For example, for the major splice donor site in HIV-1 viral vector genomes this can be T¹G²/G³T⁴, wherein “/” is the cleavage site. For example, for the cryptic splice donor site in HIV-1 viral vector genomes this can be G¹A²/G³T⁴, wherein “/” is the cleavage site. Additionally, the point mutation(s) can be introduced adjacent to a splice donor site. For example, the point mutation can be introduced upstream or downstream of a splice donor site. In embodiments where the nucleic acid sequence comprising a major and/or cryptic splice donor site is mutated by introducing multiple point mutations therein, the point mutations can be introduced upstream and/or downstream of the cryptic splice donor site.

As described herein, the nucleotide sequence encoding the RNA genome of the lentiviral vector for use according to the invention may optionally further comprise a mutation in a cryptic splice donor site within the SL4 loop of the packaging sequence. In one embodiment a GT dinucleotide in said cryptic splice donor site within the SL4 loop of the packaging sequence is mutated to GC.

In one embodiment, the nucleotide sequence encoding the lentiviral vector genome may be suitable for use in a lentiviral vector in a U3 or tat-independent system for vector production. As described herein, 3^{rd} generation lentiviral vectors are U3/tat-independent, and the nucleotide sequences according to the present invention may be used in the context of a 3^{rd} generation lentiviral vector. In one embodiment of the invention tat is not provided in the lentiviral vector production system, for example tat is not provided in trans. In one embodiment the cell or vector or vector production system as described herein does not comprise the tat protein. In one embodiment of the invention HIV-1 U3 is not present in the lentiviral vector production system, for example HIV-1 U3 is not provided in cis to drive transcription of vector genome expression cassette.

In one embodiment the major splice donor site in the lentiviral vector genome is inactivated and the cryptic splice donor site 3′ to the major splice donor site is inactivated, and said nucleotide sequence is for use in a tat-independent lentiviral vector.

In one embodiment the major splice donor site in the RNA genome of the lentiviral vector is inactivated and the cryptic splice donor site 3′ to the major splice donor site is inactivated, and said nucleotide sequence is produced in the absence of tat.

In one embodiment the major splice donor site in the RNA genome of the lentiviral vector is inactivated and the cryptic splice donor site 3′ to the major splice donor site is inactivated, and said nucleotide sequence has been transcribed independently of tat.

In one embodiment the major splice donor site in the RNA genome of the lentiviral vector is inactivated and the cryptic splice donor site 3′ to the major splice donor site is inactivated, and said nucleotide sequence is for use in a U3-independent lentiviral vector.

In one embodiment the major splice donor site in the RNA genome of the lentiviral vector is inactivated and the cryptic splice donor site 3′ to the major splice donor site is inactivated, and said nucleotide sequence has been transcribed independently of the U3 promoter.

In one embodiment the major splice donor site in the RNA genome of the lentiviral vector is inactivated and the cryptic splice donor site 3′ to the major splice donor site is inactivated, and said nucleotide sequence has been transcribed by a heterologous promoter.

In one embodiment, transcription of the nucleotide sequence as described herein is not dependent on the presence of U3. The nucleotide sequence may be derived from a U3-independent transcription event. The nucleotide sequence may be derived from a heterologous promoter. A nucleotide sequence as described herein may not comprise a native U3 promoter.

### Construction of Splice Site Mutants

Splice site mutants of the present invention may be constructed using a variety of techniques. For example, mutations may be introduced at particular loci by synthesising oligonucleotides containing a mutant sequence, flanked by restriction sites enabling ligation to fragments of the native sequence. Following ligation, the resulting reconstructed sequence comprises a derivative having the desired nucleotide insertion, substitution, or deletion. By way of further example, splice site mutants may be constructed as described in WO 2021/160993 (which is incorporated herein by reference in its entirety).

Other known techniques allowing alterations of DNA sequence include recombination approaches such as Gibson assembly, Golden-gate cloning and In-fusion.

Alternatively, oligonucleotide-directed site-specific (or segment specific) mutagenesis procedures may be employed to provide an altered sequence according to the substitution, deletion, or insertion required. Deletion or truncation derivatives of splice site mutants may also be constructed by utilising convenient restriction endonuclease sites adjacent to the desired deletion. Subsequent to restriction, overhangs may be filled in, and the DNA religated. Exemplary methods of making the alterations set forth above are disclosed by Sambrook et al. (Molecular cloning: A Laboratory Manual, 2d Ed., Cold Spring Harbor Laboratory Press, 1989).

Splice site mutants may also be constructed utilising techniques of PCR mutagenesis, chemical mutagenesis, chemical mutagenesis (Drinkwater and Klinedinst, 1986) by forced nucleotide misincorporation (e.g., Liao and Wise, 1990), or by use of randomly mutagenised oligonucleotides (Horwitz et al., 1989).

The present invention also provides a method for producing a lentiviral vector nucleotide sequence, comprising the steps of:

providing a nucleotide sequence encoding the RNA genome of a lentiviral vector as described herein; and mutating the major splice donor site and cryptic splice donor site as described herein in said nucleotide sequence.

Combination with Modified U1 snRNA to Improve Vector Titre

MSD-mutated lentiviral vectors are preferable to current standard lentiviral vectors for use as gene therapy vectors due to their reduced capacity to partake in aberrant splicing events both during LV production and in target cells. However, the production of MSD mutated vectors has either relied upon supply of the HIV-1 tat protein (1^{st} and 2^{nd} generation U3-dependent lentiviral vectors), has been of lower efficiency due to the destabilising effect of mutating the MSD on vector RNA levels (in 3^{rd} generation vectors), or, as discovered by the present inventors, is improved by co-expression of modified U1 snRNA. The present inventors have previously found that MSD-mutated, 3^{rd} generation (i.e. U3/tat-independent) LVs could be produced to high titre by co-expression of a modified U1 snRNA directed to bind to the 5′packaging region of the vector genome RNA during production (see WO 2021/014157 and WO 2021/160993, incorporated herein by reference).

The amount of vRNA produced from so-called MSD-mutated (or MSD-2KO) lentiviral vector genomes is typically substantially reduced, leading to lower vector titres. It is theorized that an ‘early’ interaction with the MSD and U1 snRNA (prior to splicing decisions) is important for transcription elongation from the external promoter. The inventors previously found that one solution to this problem was to provide a modified U1 snRNA in trans during LV production to stabilize the vRNA (see WO 2021/014157 and WO 2021/160993).

These modified U1 snRNAs can enhance the production titres of MSD-mutated LVs in a manner that is independent of the presence of the 5′polyA signal within the 5′R region, indicating a novel mechanism over others' use of modified U1 snRNAs to suppress polyadenylation (so called U1-interference, [Ui]). Targeting the modified U1 snRNAs to critical sequences of the packaging region produced the greatest enhancement in MSD-mutated LV titres.

The present inventors previously showed that the output titres of lentiviral vectors can be enhanced by co-expressing non-coding RNAs based on U1 snRNAs, which have been modified so that they no longer target the endogenous sequence (a splice donor site) but now target a sequence within the vRNA molecule. As demonstrated in WO 2021/014157 and WO 2021/160993, the relative enhancement in output titres of lentiviral vectors harbouring attenuating mutations within the major splice donor region (containing the major splice donor and cryptic splice donor sites) by said modified U1 snRNAs are greater than standard lentiviral vectors containing a non-mutated major splice donor region.

Vector genomes harbouring a broad range of mutation types within the major splice donor region (point mutations, region deletion, and sequence replacement) that lead to reduced titres may be used in combination with a modified U1 snRNA. The approach may comprise co-expression of modified U1 snRNAs together with the other vector components during vector production. The modified U1 snRNAs are designed such that binding to the consensus splice donor site has been ablated by replacing it with a heterologous sequence that is complementary to a target sequence within the vector genome vRNA.

In some embodiments, the nucleotide sequence of the invention is used in combination with a modified U1 snRNA.

In some embodiments, the nucleotide sequence of the invention further comprises a nucleotide sequence encoding a modified U1 snRNA.

In some embodiments, the nucleotide sequence encoding the lentiviral vector genome further encodes a modified U1 snRNA.

In some embodiments, the nucleotide sequence encoding the lentiviral vector genome is operably linked to the nucleotide sequence encoding the modified U1 snRNA.

In some embodiments, wherein said modified U1 snRNA has been modified to bind to a nucleotide sequence within the packaging region of the lentiviral vector genome.

In one embodiment, the nucleotide sequence encoding a modified U1 snRNA may be provided on a different nucleotide sequence, for example on a different plasmid. In other words, the nucleotide sequence encoding a modified U1 snRNA may be provided in trans during production of a lentiviral vector as described herein.

Splicing and polyadenylation are key processes for mRNA maturation, particularly in higher eukaryotes where most protein-coding transcripts contain multiple introns. The elements within a pre-mRNA that are required for splicing include the 5′ splice donor signal, the sequence surrounding the branch point and the 3′ splice acceptor signal. Interacting with these three elements is the spliceosome, which is formed by five small nuclear RNAs (snRNAs), including U1 snRNA, and associated nuclear proteins (snRNP). U1 snRNA is expressed by a polymerase II promoter and is present in most eukaryotic cells (Lund et al., 1984, J. Biol. Chem., 259:2013-2021). Human U1 snRNA (small nuclear RNA) is 164 nt long with a well-defined structure consisting of four stem-loops (West, S., 2012, Biochemical Society Transactions, 40:846-849). U1 snRNA contains a short sequence at its 5′-end that is broadly complementary to the 5′ splice donor sites at exon-intron junctions. U1 snRNA participates in splice-site selection and spliceosome assembly by base pairing to the 5′ splice donor site. A known function for U1 snRNA outside splicing is in the regulation of 3′-end mRNA processing: it suppresses premature polyadenylation (polyA) at early polyA signals (particularly within introns).

Human U1 snRNA (small nuclear RNA) is 164 nt long with a well-defined structure consisting of four stem-loops. The endogenous non-coding RNA, U1 snRNA, binds to the consensus 5′ splice donor site (e.g. 5′-MAGGURR-3′, wherein M is A or C and R is A or G) via the native splice donor annealing sequence (e.g. 5′-ACUUACCUG-3′) during early steps of intron splicing. Stem loop I binds to U1A-70K protein that has been shown to be important for polyA suppression. Stem loop II binds to U1A protein, and the 5′-AUUUGUGG-3′ sequence binds to Sm proteins, which together with Stem loop IV, is important for U1 snRNA processing. As defined herein, the modified U1 snRNA for use according to the present invention is modified to introduce a heterologous sequence that is complementary to a target sequence within the vector genome vRNA molecule at the site of the native splice donor targeting/annealing sequence.

Suitable modified U1 snRNAs for use according to the present invention are described in WO 2021/014157 and WO 2021/160993 and are incorporated herein by reference.

The modified U1 snRNAs can be manufactured according to methods generally known in the art. For example, the modified U1 snRNAs can be manufactured by chemical synthesis or recombinant DNA/RNA technology. By way of further example, the modified U1 snRNAs as described herein can be manufactured as described in WO 2021/014157 and WO 2021/160993.

The introduction of a nucleotide sequence encoding a modified U1 snRNA as described herein into a cell using conventional molecular and cell biology techniques is within the capabilities of a person of ordinary skill in the art.

### TRIP System

The present invention, as disclosed herein, may be combined with the ‘TRIP’ system.

The expression of transgenes during viral vector production can be detrimental to vector yields and/or quality by negatively affecting one or more aspects of the production phase. For example, a transgene protein may be cytotoxic or may, directly or indirectly, impair vector virion assembly and/or infectivity, or its presence during downstream processing or in the final product may be problematic. Thus, expression of the protein encoded by the NOI within viral vector production cells can adversely affect therapeutic vector titres (as shown in WO2015/092440). In addition, an NOI encoding a transmembrane POI may, for example, lead to high surface expression of the transmembrane protein in the viral vector virion, potentially altering the physical properties of the virions. Furthermore, this incorporation may present the POI to the patient's immune system at the site of delivery, which may negatively affect transduction and/or the long-term expression of the therapeutic gene in vivo.

WO2015/092440 and WO2021/094752, which are incorporated in their entirety herein by reference, disclose the use of a heterologous translation control system in eukaryotic cell cultures to repress the translation of the NOI (repress transgene expression) during viral vector production and thus repress or prevent expression of the protein encoded by the NOI. This system is referred to as the Transgene Repression In vector Production cell system or TRIP system.

In one form, the TRIP system utilises the bacterial trp operon regulation protein, tryptophan RNA-binding attenuation protein (TRAP), and the TRAP binding site/sequence (tbs) to mediate transgene repression. The use of this system does not impede the production of packageable vector genome molecules nor the activity of vector virions, and does not interfere with the long-term expression of the NOI in the target cell.

The term “binding site” is to be understood as a nucleic acid sequence that is capable of interacting with a certain protein.

By “capable of interacting” it is to be understood that the nucleic acid binding site (e.g. tbs or portion thereof) is capable of binding to a protein, for example TRAP, under the conditions that are encountered in a cell, for example a eukaryotic viral vector production cell. Such an interaction with an RNA-binding protein such as TRAP results in the repression or prevention of translation of a NOI to which the nucleic acid binding site (e.g. the tbs or portion thereof) is operably linked.

A consensus TRAP binding site sequence that is capable of binding TRAP is [KAGNN] repeated multiple times (e.g. 6, 7, 8, 9, 10, 11, 12 or more times); such sequence is found in the native trp operon. In the native context, occasionally AAGNN is tolerated and occasionally additional “spacing” N nucleotides result in a functional sequence. In vitro experiments have demonstrated that at least 6 or more consensus repeats are required for TRAP-RNA binding (Babitzke P, Y. J., Campanelli D. (1996) Journal of Bacteriology 178 (17): 5159-5163). Therefore, preferably there are 6 or more continuous [KAGN_{≥2}] sequences present within the tbs, wherein “K” may be T or G in DNA and U or G in RNA and “N” is to be understood as specifying any nucleotide at that position in the sequence (for example, “N” could be G, A, T, C or U).

In some embodiments, the lentiviral vector genome further comprises a tbs.

In some embodiments, the nucleotide sequence of the invention further comprises a TRAP binding site (tbs).

In some embodiments, a nucleotide sequence encoding TRAP is present during production of the lentiviral vector as described herein.

Suitable tbs are described in WO2015/092440 and WO2021/094752 and are incorporated herein by reference. Suitably, the nucleotide sequence may further comprise a tbs, and also may comprise a Kozak sequence, wherein said tbs overlaps the Kozak sequence, or wherein said Kozak sequence comprises a portion of a tbs. Suitably, the nucleotide sequence may further comprise a multiple cloning site and a Kozak sequence, wherein said multiple cloning site is overlapping with or located downstream to the 3′ KAGN2-3 repeat of the tbs and upstream of the Kozak sequence.

As used herein, a “multiple cloning site” is to be understood as a DNA region which contains several restriction enzyme recognition sites (restriction enzyme sites) very close to each other. The RE sites may be overlapping in the MCS for use in the invention.

As used herein, a “restriction enzyme site” or “restriction enzyme recognition site” is a location on a DNA molecule containing specific sequences of nucleotides, 4-8 nucleotides in length, which are recognised by restriction enzymes. A restriction enzyme recognises a specific RE site (i.e. a specific sequence) and cleaves the DNA molecule within, or nearby, the RE site.

In some embodiments of the present invention, the nucleotide of interest (i.e. transgene) is operably linked to the tbs. In some embodiments, the nucleotide of interest is translated in a target cell which expresses TRAP. In some embodiments, the nucleotide of interest is translated in a target cell which lacks TRAP.

By “operably linked” it is to be understood that the components described are in a relationship permitting them to function in their intended manner. Therefore a tbs or portion thereof for use in the invention operably linked to a NOI is positioned in such a way that translation of the NOI is modified when as TRAP binds to the tbs or portion thereof.

The tbs may be capable of interacting with TRAP such that translation of the nucleotide of interest is repressed or prevented in a viral vector production cell.

### Viral Cis-Acting Sequences

ORFs present in the vector backbone delivered in transduced (e.g. patient) cells could be transcribed, for example, when read-through transcription from upstream cellular promoters occurs (lentiviral vectors target active transcription sites), leading to potential aberrant transcription of genetic material located in the vector backbone in patient cells. This potential aberrant transcription of genetic material located in the vector backbone following read-through transcription could also occur during lentiviral vector production in production cells.

The viral cis-acting sequence present within lentiviral vector genomes may contain multiple internal ORFs. These internal ORFs may be found between an internal ATG sequence of the viral cis-acting sequence and the stop codon immediately 3′ to the ATG sequence.

Modifications in a viral cis-acting sequence to disrupt at least one internal ORF, for example by mutating the ATG sequence which denotes the start of the at least one internal ORF, are tolerated. Thus, the modified viral cis-acting sequence described herein retains its function.

Accordingly, in some embodiments of the present invention, the lentiviral vector genome comprises at least one modified viral cis-acting sequence, wherein at least one internal open reading frame (ORF) in the viral cis-acting sequence is disrupted (see WO 2021/181108 A¹, incorporated herein by reference in its entirety). The at least one internal ORF may be disrupted by mutating at least one ATG sequence (ATG sequences may function as translation initiation codons).

In some embodiments of the present invention, the lentiviral vector genome comprises a modified nucleotide sequence encoding gag, wherein at least one internal ORF in the modified nucleotide sequence encoding gag is disrupted (see WO 2021/181108 A1, incorporated herein by reference in its entirety). The at least one internal ORF in the modified nucleotide sequence encoding gag may be disrupted by mutating at least one ATG sequence as described herein.

Suitable modified viral cis-acting sequences and modified nucleotide sequences encoding gag for use according to the present invention are described in WO 2021/181108 A1 and are incorporated herein by reference.

In some embodiments, the lentiviral vector genome comprises at least two (suitably at least three, at least four, at least five, at least six, at least seven) modified viral cis-acting sequences.

In some embodiments, at least two (suitably at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty) internal ORFs in the at least one viral cis-acting sequence and/or in the nucleotide sequence encoding gag may be disrupted. In some embodiments, at least three internal ORFs in the at least one viral cis-acting sequence and/or in the nucleotide sequence encoding gag may be disrupted.

In some embodiments, one (suitably, two, three, four, five, six, seven, eight, nine, ten, eleven, twelve, thirteen, fourteen, fifteen, sixteen, seventeen, eighteen, nineteen or twenty) internal ORFs in the at least one viral cis-acting sequence and/or the nucleotide sequence encoding gag may be disrupted.

In some embodiments, the at least one internal ORF may be disrupted such that the internal ORF is not expressed. In some embodiments, the at least one internal ORF may be disrupted such that the internal ORF is not translated. In some embodiments, the at least one internal ORF may be disrupted such that no protein is expressed from the internal ORF. In some embodiments, the at least one internal ORF may be disrupted such that no protein is translated from the internal ORF. Thus, the at least one internal ORF present in the modified viral cis-acting sequence and/or in the modified nucleotide sequence encoding gag in the vector backbone delivered in transduced cells may be disrupted such that aberrant transcription of the internal ORF is prevented when there is read-through transcription from upstream cellular promoters.

In one embodiment, the at least one internal ORF may be disrupted by mutating at least one ATG sequence. A “mutation” of an ATG sequence may comprise one or more nucleotide deletions, additions, or substitutions.

In one embodiment, the at least one ATG sequence may be mutated in the modified viral cis-acting sequence and/or in the modified nucleotide sequence encoding gag to a sequence selected from the group consisting of:
a) an ATTG sequence;
b) an ACG sequence;
c) an A-G sequence;
d) an AAG sequence;
e) a TTG sequence; and/or
f) an ATT sequence.

The at least one ATG sequence may be mutated to an ATTG sequence in the modified viral cis-acting sequence and/or the modified nucleotide sequence encoding gag. The at least one ATG sequence may be mutated to an ACG sequence in the modified viral cis-acting sequence and/or the modified nucleotide sequence encoding gag. The at least one ATG sequence may be mutated to an A-G sequence in the modified viral cis-acting sequence and/or the modified nucleotide sequence encoding gag. The at least one ATG sequence may be mutated to an AAG sequence in the modified viral cis-acting sequence and/or the modified nucleotide sequence encoding gag. The at least one ATG sequence may be mutated to a TTG sequence in the modified viral cis-acting sequence and/or the modified nucleotide sequence encoding gag. The at least one ATG sequence may be mutated to an ATT sequence in the modified viral cis-acting sequence and/or the modified nucleotide sequence encoding gag.

In one embodiment, the at least one modified viral cis-acting element and/or the modified nucleotide sequence encoding gag may lack ATG sequences.

In some embodiments, all ATG sequences within viral cis-acting sequences and/or the nucleotide sequence encoding gag in the lentiviral vector genome are mutated.

Lentiviral vectors typically comprise multiple viral cis-acting sequences. Example viral cis-acting sequences include gag-p17, Rev response element (RRE), central polypurine tract (cppt) and Woodchuck hepatitis virus (WHV) post-transcriptional regulatory element (WPRE).

In some embodiments, the at least one viral cis-acting sequence may be at least one lentiviral cis-acting sequence. Example lentiviral cis-acting sequences include the RRE and cppt.

In some embodiments, the at least one viral cis-acting sequence may be at least one non-lentiviral cis-acting sequence.

In some embodiments, the at least one viral cis-acting sequence may be at least one lentiviral cis-acting sequence and at least one non-lentiviral cis-acting sequence.

In some embodiments, the at least one viral cis-acting sequence is:
a) gag-p17; and/or
b) a Rev response element (RRE); and/or
c) a Woodchuck hepatitis virus (WHV) post-transcriptional regulatory element (WPRE).

In some embodiments, the at least one viral cis-acting sequence is a RRE.

In some embodiments, the at least one viral cis-acting sequence is a WPRE.

In some embodiments, the lentiviral vector genome comprises at least two (suitably, at least 3, at least 4, at least 5) modified viral cis-acting sequences.

In some embodiments, the lentiviral vector genome comprises a modified RRE as described herein and a modified WPRE as described herein.

In some embodiments, the lentiviral vector genome comprises a modified RRE as described herein, a modified WPRE as described herein and a modified nucleotide sequence encoding gag as described herein.

In one embodiment, the lentiviral vector genome as described herein lacks ATG sequences in the backbone of the vector genome. In one embodiment, the lentiviral vector genome as described herein lacks ATG sequences except in the NOI (i.e. transgene).

In one embodiment, the lentiviral vector genome comprises at least one modified viral cis-acting sequence and/or a modified nucleotide sequence encoding gag, wherein at least one internal open reading frame (ORF) in the viral cis-acting sequence or in the nucleotide sequence encoding gag is ablated.

In one embodiment, the lentiviral vector genome comprises at least one modified viral cis-acting sequence and/or a modified nucleotide sequence encoding gag, wherein at least one internal open reading frame (ORF) in the viral cis-acting sequence or in the nucleotide sequence encoding gag is silenced.

### Modified Gag and Viral Gag-p17 Protein

A further preferred but optional feature of the invention is the minimization of gag sequences included within the packaging sequences used in combination with the aforementioned features. The amount of gag typically included within HIV-1 lentiviral vector packaging sequences can be reduced by at least 270 nucleotides, but may be reduced by up to the entire gag sequence. The deleted gag nucleotide sequence may be that of the gag-p17 instability sequence. Deletion of the gag-p17 instability sequence typically results in reduced vector titres unless the first ATG codon of the remaining gag sequence is mutated.

In some embodiments the reduced packaging sequences comprise deleted gag sequences wherein only the first 80 nucleotides of gag remain.

In some embodiment the reduced packaging sequences comprise deleted gag sequences wherein only the first 70 nucleotides of gag remain.

In some embodiment the reduced packaging sequences comprise deleted gag sequences wherein only the first 60 nucleotides of gag remain.

In some embodiment the reduced packaging sequences comprise deleted gag sequences wherein only the first 50 nucleotides of gag remain.

In some embodiment the reduced packaging sequences comprise deleted gag sequences wherein only the first 40 nucleotides of gag remain.

In some embodiment the reduced packaging sequences comprise deleted gag sequences wherein only the first 30 nucleotides of gag remain.

In some embodiment the reduced packaging sequences comprise deleted gag sequences wherein only the first 20 nucleotides of gag remain.

In some embodiment the reduced packaging sequences comprise deleted gag sequences wherein only the first 10 nucleotides of gag remain.

In some embodiment the reduced packaging sequences comprise deleted gag sequences wherein no nucleotides of gag remain.

In some embodiment, nucleotide sequences of the invention comprise ablated gag sequences wherein the gag sequences comprise only up to the first 10, up to the first 20, up to the first 30, up to the first 40, up to the first 50, up to the first 60, up to the first 70, or up to the first 80 nucleotides of gag.

The nucleotide sequence encoding gag may be a truncated nucleotide sequence encoding a part of gag. The nucleotide sequence encoding gag may be a minimal truncated nucleotide sequence encoding a part of gag. The part of gag may be a contiguous sequence. The truncated nucleotide sequence or minimal truncated nucleotide sequence encoding a part of gag may also contain at least one frameshift mutation.

An example truncated nucleotide sequence encoding a part of gag and which contains a frameshift mutation at position 45-46 is as follows:

(SEQ ID NO: 17)
ATGGGTGCGAGAGCGTCAGTATTAAGCGGGGGAGAATTAGATCGCGATGG
GAAAAAATTCGGTTAAGGCCAGGGGGAAAGAAAAAATATAAATTAAAACA
TATAGTATGGGCAAGCAGGGAGCTAGAACGATTCGCAGTTAATCCTGGCC
TGTTAGAAACATCAGAAGGCTGTAGACAAATACTGGGACAGCTACAACCA
TCCCTTCAGACAGGATCAGAAGAACTTAGATCATTATATAATACAGTAGC
AACCCTCTATTGTGTGCATCAAAGGATAGAGATAAAAGACACCAAGGAAG
CTTTAGACAAGATAGAGGGAGAGCAAAACAAAAGTAAGA.

An example minimal truncated nucleotide sequence encoding a part of gag and which contains a frameshift mutation at position 45-46 is as follows:

(SED ID NO: 18)
ATGGGTGCGAGAGCGTCAGTATTAAGCGGGGGAGAATTAGATCGCGATGG
GAAAAAATTCGGTTAAGGCCAGGGGGAAAGA.

The nucleotide sequence encoding gag may, for example, comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 17; or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 18.

The modified nucleotide sequence encoding gag may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 17; or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 18.

The modified nucleotide sequence encoding gag may comprise the sequence as set forth in SEQ ID NO: 17 or SEQ ID NO: 18, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein at least one ATG sequence selected from (a) to (c) is mutated:
a) ATG corresponding to positions 1-3 of SEQ ID NO: 17;
b) ATG corresponding to positions 47-49 of SEQ ID NO: 17; and/or
c) ATG corresponding to positions 107-109 of SEQ ID NO: 17.

An example modified truncated nucleotide sequence encoding part of gag and which contains a frameshift mutation is as follows:

(SEQ ID NO: 19)
ACGGGTGCGAGAGCGTCAGTATTAAGCGGGGGAGAATTAGATCGCGATTG
GGAAAAAATTCGGTTAAGGCCAGGGGGAAAGAAAAAATATAAATTAAAAC
ATATAGTATTGGGCAAGCAGGGAGCTAGAACGATTCGCAGTTAATCCTGG
CCTGTTAGAAACATCAGAAGGCTGTAGACAAATACTGGGACAGCTACAAC
CATCCCTTCAGACAGGATCAGAAGAACTTAGATCATTATATAATACAGTA
GCAACCCTCTATTGTGTGCATCAAAGGATAGAGATAAAAGACACCAAGGA
AGCTTTAGACAAGATAGAGGGAGAGCAAAACAAAAGTAAGA.

An example modified minimal truncated nucleotide sequence encoding a part of gag and which contains a frameshift mutation is as follows:

(SED ID NO: 20)
ACGGGTGCGAGAGCGTCAGTATTAAGCGGGGGAGAATTAGATCGCGATTG
GGAAAAAATTCGGTTAAGGCCAGGGGGAAAGA.

The modified nucleotide sequence encoding gag may comprise the sequence as set forth in SEQ ID NO: 19, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than three (suitably less than two or less than one) ATG sequences.

The modified nucleotide sequence encoding gag may comprise the sequence as set forth in SEQ ID NO: 20, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than two (suitably less than one) ATG sequences.

The modified nucleotide sequence encoding gag may comprise less than three ATG sequences. Suitably, the modified nucleotide sequence encoding gag may comprise less than two or less than one ATG sequence(s). The modified nucleotide sequence encoding gag may lack an ATG sequence.

Lentiviral vector genomes lacking a nucleotide sequence encoding Gag-p17 or a fragment thereof are described in WO 2021/181108 A1, incorporated herein by reference in its entirety. Such lentiviral vector genomes as described in WO 2021/181108 are suitable for use according to the present invention.

The lentiviral vector genome as described herein may lack a nucleotide sequence encoding Gag-p17 or a fragment thereof. The lentiviral vector genome may, for example, not express Gag-p17 or a fragment thereof. In one embodiment, the lentiviral vector genome may lack the sequence as set forth in SEQ ID NO: 13.

The viral protein Gag-p17 surrounds the capsid of the lentiviral vector particle, and is in turn surrounded by the envelope protein. A nucleotide sequence encoding Gag-p17 has historically been included in lentiviral vector genomes for the production of therapeutic lentiviral vectors. The nucleotide sequence encoding Gag-p17 present within lentiviral vector genomes is typically embedded within the packaging region containing highly structured RNA towards the 5′ region of the RNA (the 5′UTR). The nucleotide sequence encoding Gag-p17 typically comprises an RNA instability sequence (INS), herein referred to as p17-INS.

Deletion of p17-INS from the backbone of the lentiviral vector genome does not significantly impact vector titres during lentiviral vector production.

The lentiviral vector genome lacking a nucleotide sequence encoding Gag-p17 or p17-INS is of a smaller size compared to a lentiviral vector genome comprising a nucleotide sequence encoding Gag-p17 or p17-INS. Thus, the amount of viral DNA contained within the viral vector backbone delivered in transduced cells is reduced when a lentiviral vector genome lacking a nucleotide sequence encoding Gag-p17 or p17-INS is used. Further, the lentiviral vector genome lacking a nucleotide sequence encoding Gag-p17 or p17-INS may be used to deliver a transgene of larger size than the transgenes which can be delivered using a lentiviral vector genome containing a nucleotide sequence encoding Gag-p17 or p17-INS. Therefore, there are several reasons why it may be desirable to delete nucleotide sequence encoding Gag-p17 or p17-INS within the vector backbone. Deletion of gag sequences in order to reduce the size of lentiviral vector genome sequences has been reported (Sertkaya, H., et al., Sci Rep 11:12067 (2021)).

In some embodiments, the lentiviral vector genome lacks either (i) a nucleotide sequence encoding Gag-p17 or (ii) a fragment of a nucleotide sequence encoding Gag-p17.

In some embodiments, the lentiviral vector genome lacks a nucleotide sequence encoding p17-INS or a fragment thereof.

An example p17-INS is as follows:

(SEQ ID NO: 21)
AAAAATATAAATTAAAACATATAGTATGGGCAAGCAGGGAGCTAGAACGA
TTCGCAGTTAATCCTGGCCTGTTAGAAACATCAGAAGGCTGTAGACAAAT
ACTGGGACAGCTACAACCATCCCTTCAGACAGGATCAGAAGAACTTAGAT
CATTATATAATACAGTAGCAACCCTCTATTGTGTGCATCAAAGGATAGAG
ATAAAAGACACCAAGGAAGCTTTAGACAAGATAGAGGGAGAGCAAAACAA
AAGTA.

In one embodiment, the lentiviral vector genome may lack the sequence as set forth in SEQ ID NO: 21.

In one embodiment, the fragment of a nucleotide sequence encoding Gag-p17 is a part of a full-length nucleotide sequence encoding Gag-p17. In one embodiment, the fragment comprises or consists of at least about 10 nucleotides (suitably at least about 20, at least about 30, at least about 40, at least about 50, at least about 100, at least about 150, at least about 200, at least about 250, at least about 300, at least about 350 nucleotides).

In one embodiment, the fragment of a nucleotide sequence encoding Gag-p17 may have a length which is between 1% and 99% of full-length nucleotide sequence encoding Gag-p17. Suitably, the fragment of a nucleotide sequence encoding Gag-p17 may have a length which is at least about 10% (suitably at least about 20%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%) of a full-length nucleotide sequence encoding Gag-p17, such as a native nucleotide sequence encoding Gag-p17. The fragment may be a contiguous region of a full-length nucleotide sequence encoding Gag-p17, such as a native nucleotide sequence encoding Gag-p17.

In one embodiment, the fragment of a nucleotide sequence encoding Gag-p17 may have a length which is between 1% and 99% of full-length nucleotide sequence encoding p17-INS. Suitably, the fragment of a nucleotide sequence encoding Gag-p17 may have a length which is at least about 10% (suitably at least about 20%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%) of a full-length nucleotide sequence encoding p17-INS, such as a native nucleotide sequence encoding p17-INS (e.g. SEQ ID NO: 21). The fragment may be a contiguous region of a full-length nucleotide sequence encoding p17-INS, such as a native nucleotide sequence encoding p17-INS (e.g. SEQ ID NO: 21).

In one embodiment, the fragment of a nucleotide sequence encoding Gag-p17 comprises or consists of the INS located in the nucleotide sequence encoding Gag-p17.

In one embodiment, the lentiviral vector genome lacking either (i) a nucleotide sequence encoding Gag-p17 or (ii) a fragment of a nucleotide sequence encoding Gag-p17 comprises at least one modified viral cis-acting sequence as described herein.

In one embodiment, the lentiviral vector genome lacking either (i) a nucleotide sequence encoding Gag-p17 or (ii) a fragment of a nucleotide sequence encoding Gag-p17 may comprise a modified RRE, a modified WPRE and/or a modified nucleotide sequence encoding gag as described herein.

In one embodiment, the lentiviral vector genome lacking either (i) a nucleotide sequence encoding Gag-p17 or (ii) a fragment of a nucleotide sequence encoding Gag-p17 may comprise a modified RRE as described herein, a modified WPRE as described herein and a modified nucleotide sequence encoding gag as described herein.

### Modified Rev Response Element (RRE)

In some embodiments, the lentiviral vector genome comprises a modified Rev response element (RRE), wherein at least one internal open reading frame (ORF) in the RRE is disrupted as described herein.

The RRE is an essential viral RNA element that is well conserved across lentiviral vectors and across different wild-type HIV-1 isolates. The RRE present within lentiviral vector genomes may contain multiple internal ORFs. These internal ORFs may be found between an internal ATG sequence of the RRE and the stop codon immediately 3′ to the ATG sequence.

The RRE present within lentiviral vector genomes is typically embedded within the packaging region containing highly structured RNA towards the 5′ region of the RNA (the 5′UTR). The 5′ UTR structure consists of series of stem-loop structures connected by small linkers. These stem-loops include the RRE. Thus, the RRE itself has a complex secondary structure, involving complementary base-pairing, to which Rev binds.

Modifications in the RRE to disrupt at least one internal ORF, for example by mutating the ATG sequence which denotes the start of the at least one internal ORF, are tolerated. Thus, the modified RREs described herein retain Rev binding capacity.

The modified RRE may comprise less than eight ATG sequences.

Accordingly, in some embodiments, the lentiviral vector genome comprises a modified Rev response element (RRE), wherein the modified RRE comprises less than eight ATG sequences.

Suitably, the modified RRE may comprise less than seven, less than six, less than five, less than four, less than three, less than two or less than one ATG sequence(s). The modified RRE may lack an ATG sequence.

The RRE may be a minimal functional RRE. An example minimal functional RRE is as follows:

(SEQ ID NO: 22)
AGGAGCTTTGTTCCTTGGGTTCTTGGGAGCAGCAGGAAGCACTATGGGCG
CAGCGTCAATGACGCTGACGGTACAGGCCAGACAATTATTGTCTGGTATA
GTGCAGCAGCAGAACAATTTGCTGAGGGCTATTGAGGCGCAACAGCATCT
GTTGCAACTCACAGTCTGGGGCATCAAGCAGCTCCAGGCAAGAATCCTGG
CTGTGGAAAGATACCTAAAGGATCAACAGCTCCT.

By “minimal functional RRE” or “minimal RRE” is meant a truncated RRE sequence which retains the function of the full-length RRE. Thus, the minimal functional RRE retains Rev binding capacity.

The RRE may be the core RRE. An example core RRE is as follows:

(SEQ ID NO: 23)
GGAGCTTTGTTCCTTGGGTTCTTGGGAGCAGCAGGAAGCACTATGGGCGC
AGCGTCAATGACGCTGACGGTACAGGCCAGACAATTATTGTCTGGTATAG
TGCAGCAGCAGAACAATTTGCTGAGGGCTATTGAGGCGCAACAGCATCTG
TTGCAACTCACAGTCTGGGGCATCAAGCAGCTCCAGGCAAGAATCCTGGC
TGTGGAAAGATACCTAAAGGATCAACAGCTCCTGGGGATTTGGGGTTGCT
CTGGAAAACTCATTTGCACCACTGCTGTGCCTTGGAATGCTAGTTGGAGT
AATAAATCTCTGGAACAGATTTGGAATCACACGACCTGGATGGAGTGGGA
C

The RRE may be a full-length RRE. An example full-length RRE is as follows:

(SEQ ID NO: 24)
TGATCTTCAGACCTGGAGGAGGAGATATGAGGGACAATTGGAGAAGTGAA
TTATATAAATATAAAGTAGTAAAAATTGAACCATTAGGAGTAGCACCCAC
CAAGGCAAAGAGAAGAGTGGTGCAGAGAGAAAAAAGAGCAGTGGGAATAG
GAGCTTTGTTCCTTGGGTTCTTGGGAGCAGCAGGAAGCACTATGGGCGCA
GCGTCAATGACGCTGACGGTACAGGCCAGACAATTATTGTCTGGTATAGT
GCAGCAGCAGAACAATTTGCTGAGGGCTATTGAGGCGCAACAGCATCTGT
TGCAACTCACAGTCTGGGGCATCAAGCAGCTCCAGGCAAGAATCCTGGCT
GTGGAAAGATACCTAAAGGATCAACAGCTCCTGGGGATTTGGGGTTGCTC
TGGAAAACTCATTTGCACCACTGCTGTGCCTTGGAATGCTAGTTGGAGTA
ATAAATCTCTGGAACAGATTTGGAATCACACGACCTGGATGGAGTGGGAC
AGAGAAATTAACAATTACACAAGCTTAATACACTCCTTAATTGAAGAATC
GCAAAACCAGCAAGAAAAGAATGAACAAGAATTATTGGAATTAGATAAAT
GGGCAAGTTTGTGGAATTGGTTTAACATAACAAATTGGCTGTGGTATATA
AAATTATTCATAATGATAGTAGGAGGCTTGGTAGGTTTAAGAATAGTTTT
TGCTGTACTTTCTATAGTGAATAGAGTTAGGCAGGGATATTCACCATTAT
CGTTTCAGACCCACCTCCCAACCCCGAGGGGAC.

The RRE may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 22;
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 23; and/or
c) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 24.

The modified RRE may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 22;
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 23; and/or
c) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 24.

The modified RRE may comprise the sequence as set forth in SEQ ID NO: 22, SEQ ID NO: 23 or SEQ ID NO: 24, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein at least one ATG sequence selected from the group (a)-(h) is mutated:
a) ATG corresponding to positions 27-29 of SEQ ID NO: 24;
b) ATG corresponding to positions 192-194 of SEQ ID NO: 24;
c) ATG corresponding to positions 207-209 of SEQ ID NO: 24;
d) ATG corresponding to positions 436-438 of SEQ ID NO: 24;
e) ATG corresponding to positions 489-491 of SEQ ID NO: 24;
f) ATG corresponding to positions 571-573 of SEQ ID NO: 24;
g) ATG corresponding to positions 599-601 of SEQ ID NO: 24; and/or
h) ATG corresponding to positions 663-665 of SEQ ID NO: 24.

An example modified RRE sequence is as follows:

(SEQ ID NO: 25)
AGGAGCTTTGTTCCTTGGGTTCTTGGGAGCAGCAGGAAGCACTATTGGG
CGCAGCGTCAATTGACGCTGACGGTACAGGCCAGACAATTATTGTCTGG
TATAGTGCAGCAGCAGAACAATTTGCTGAGGGCTATTGAGGCGCAACAG
CATCTGTTGCAACTCACAGTCTGGGGCATCAAGCAGCTCCAGGCAAGAA
TCCTGGCTGTGGAAAGATACCTAAAGGATCAACAGCTCCT.

A further example modified RRE sequence is as follows:

(SEQ ID NO: 26)
GGAGCTTTGTTCCTTGGGTTCTTGGGAGCAGCAGGAAGCACTATTGGGCG
CAGCGTCAATTGACGCTGACGGTACAGGCCAGACAATTATTGTCTGGTAT
AGTGCAGCAGCAGAACAATTTGCTGAGGGCTATTGAGGCGCAACAGCATC
TGTTGCAACTCACAGTCTGGGGCATCAAGCAGCTCCAGGCAAGAATCCTG
GCTGTGGAAAGATACCTAAAGGATCAACAGCTCCTGGGGATTTGGGGTTG
CTCTGGAAAACTCATTTGCACCACTGCTGTGCCTTGGAATTGCTAGTTGG
AGTAATAAATCTCTGGAACAGATTTGGAATCACACGACCTGGATTGGAGT
GGGAC

A further example modified RRE sequence is as follows:

(SEQ ID NO: 27)
TGATCTTCAGACCTGGAGGAGGAGATATTGAGGGACAATTGGAGAAGTGA
ATTATATAAATATAAAGTAGTAAAAATTGAACCATTAGGAGTAGCACCCA
CCAAGGCAAAGAGAAGAGTGGTGCAGAGAGAAAAAAGAGCAGTGGGAATA
GGAGCTTTGTTCCTTGGGTTCTTGGGAGCAGCAGGAAGCACTATTGGGCG
CAGCGTCAATTGACGCTGACGGTACAGGCCAGACAATTATTGTCTGGTAT
AGTGCAGCAGCAGAACAATTTGCTGAGGGCTATTGAGGCGCAACAGCATC
TGTTGCAACTCACAGTCTGGGGCATCAAGCAGCTCCAGGCAAGAATCCTG
GCTGTGGAAAGATACCTAAAGGATCAACAGCTCCTGGGGATTTGGGGTTG
CTCTGGAAAACTCATTTGCACCACTGCTGTGCCTTGGAATTGCTAGTTGG
AGTAATAAATCTCTGGAACAGATTTGGAATCACACGACCTGGATTGGAGT
GGGACAGAGAAATTAACAATTACACAAGCTTAATACACTCCTTAATTGAA
GAATCGCAAAACCAGCAAGAAAAGAATGAACAAGAATTATTGGAATTAGA
TAAATGGGCAAGTTTGTGGAATTGGTTTAACATAACAAATTGGCTGTGGT
ATATAAAATTATTCATAATTGATAGTAGGAGGCTTGGTAGGTTTAAGAAT
AGTTTTTGCTGTACTTTCTATAGTGAATAGAGTTAGGCAGGGATATTCAC
CATTATCGTTTCAGACCCACCTCCCAACCCCGAGGGGAC.

An example of a modified RRE sequence lacking an ATG sequence is as follows:

(SEQ ID NO: 28)
TGATCTTCAGACCTGGAGGAGGAGATATTGAGGGACAATTGGAGAAGTGA
ATTATATAAATATAAAGTAGTAAAAATTGAACCATTAGGAGTAGCACCCA
CCAAGGCAAAGAGAAGAGTGGTGCAGAGAGAAAAAAGAGCAGTGGGAATA
GGAGCTTTGTTCCTTGGGTTCTTGGGAGCAGCAGGAAGCACTATTGGGCG
CAGCGTCAATTGACGCTGACGGTACAGGCCAGACAATTATTGTCTGGTAT
AGTGCAGCAGCAGAACAATTTGCTGAGGGCTATTGAGGCGCAACAGCATC
TGTTGCAACTCACAGTCTGGGGCATCAAGCAGCTCCAGGCAAGAATCCTG
GCTGTGGAAAGATACCTAAAGGATCAACAGCTCCTGGGGATTTGGGGTTG
CTCTGGAAAACTCATTTGCACCACTGCTGTGCCTTGGAATTGCTAGTTGG
AGTAATAAATCTCTGGAACAGATTTGGAATCACACGACCTGGATTGGAGT
GGGACAGAGAAATTAACAATTACACAAGCTTAATACACTCCTTAATTGAA
GAATCGCAAAACCAGCAAGAAAAGAATtGAACAAGAATTATTGGAATTAG
ATAAATtGGGCAAGTTTGTGGAATTGGTTTAACATAACAAATTGGCTGTG
GTATATAAAATTATTCATAATTGATAGTAGGAGGCTTGGTAGGTTTAAGA
ATAGTTTTTGCTGTACTTTCTATAGTGAATAGAGTTAGGCAGGGATATTC
ACCATTATCGTTTCAGACCCACCTCCCAACCCCGAGGGGAC.

The modified RRE may comprise the sequence as set forth in SEQ ID NO: 25, SEQ ID NO: 26, SEQ ID NO: 27 or SEQ ID NO: 28, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than eight (suitably less than seven, less than six, less than five, less than four, less than three, less than two or less than one) ATG sequences.

### Modified Woodchuck Hepatitis Virus (WHV) Post-Transcriptional Regulatory Element (WPRE)

In some embodiments, the lentiviral vector genome comprises a modified Woodchuck hepatitis virus (WHV) post-transcriptional regulatory element (WPRE), wherein at least one internal open reading frame (ORF) in the WPRE is disrupted as described herein.

The WPRE can enhance expression from a number of different vector types including lentiviral vectors (U.S. Pat. Nos. 6,136,597; 6,287,814; Zufferey, R., et al. (1999) J. Virol. 73:2886-92). Without wanting to be bound by theory, this enhancement is thought to be due to improved RNA processing at the post-transcriptional level, resulting in increased levels of nuclear transcripts. A two-fold increase in mRNA stability also contributes to this enhancement (Zufferey, R., et al. ibid). The level of enhancement of protein expression from transcripts containing the WPRE versus those without the WPRE has been reported to be around 2-to-5 fold, and correlates well with the increase in transcript levels. This has been demonstrated with a number of different transgenes (Zufferey, R., et al. ibid).

The WPRE contains three cis-acting sequences important for its function in enhancing expression levels. In addition, it contains a fragment of approximately 180 bp comprising the 5′-end of the WHV X protein ORF (full length ORF is 425 bp), together with its associated promoter. The full-length X protein has been implicated in tumorigenesis (Flajolet, M. et al, (1998) J. Virol. 72:6175-6180). Translation from transcripts initiated from the X promoter results in formation of a protein representing the NH2-terminal 60 amino acids of the X protein. This truncated X protein can promote tumorigenesis, particularly if the truncated X protein sequence is integrated into the host cell genome at specific loci (Balsano, C. et al, (1991) Biochem. Biophys Res. Commun. 176:985-92; Flajolet, M. et al, (1998) J. Virol. 72:6175-80; Zheng, Y. W., et al, (1994) J. Biol. Chem. 269:22593-8; Runkel, L., et al, (1993) Virology 197:529-36). Therefore, expression of the truncated X protein could promote tumorigenesis if delivered to cells of interest, precluding safe use of wild-type WPRE sequences.

US 2005/0002907 discloses that mutation of a region of the WPRE corresponding to the X protein ORF ablates the tumorigenic activity of the X protein, thereby allowing the WPRE to be used safely in retroviral and lentiviral expression vectors to enhance expression levels of heterologous genes or nucleotides of interest.

As used herein, the “X region” of the WPRE is defined as comprising at least the first 60-amino acids of the X protein ORF, including the translation initiation codon, and its associated promoter. A “functional” X protein is defined herein as a truncated X protein that is capable of promoting tumorigenesis, or a transformed phenotype, when expressed in cells of interest. A “non-functional” X protein in the context of this application is defined as an X protein that is incapable of promoting tumorigenesis in cells of interest.

The modified WPREs described herein retain the capacity to enhance expression from the lentiviral vector.

The modified WPRE may comprise less than seven ATG sequences. The modified WPRE may comprise less than six ATG sequences.

Accordingly, in some embodiments, the lentiviral vector genome comprises a modified Woodchuck hepatitis virus (WHV) post-transcriptional regulatory element (WPRE), wherein the modified WPRE comprises less than seven ATG sequences, preferably less than six ATG sequences.

Suitably, the modified WPRE may comprise less than seven, less than six, less than five, less than four, less than three, less than two or less than one ATG sequence(s). The modified WPRE may lack ATG sequences.

In some embodiments, at least one ATG sequence in the X region of the WPRE is mutated, whereby expression of a functional X protein is prevented. In preferred embodiments, the mutation is in the translation initiation codon of the X region. As a result of the mutation of the at least one ATG sequence, the X protein may not be expressed.

In some embodiments, the modified WPRE does not comprise a mutation in an ATG sequence in the X region of the WPRE.

An example WPRE sequence is as follows:

(SEQ ID NO: 29)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCC.

An example WPRE sequence which contains a disrupted X-protein ORF is as follows:

(SEQ ID NO: 30)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCC.

The WPRE may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 29; and/or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 30.

The modified WPRE may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 29; and/or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 30.

The modified WPRE may comprise the sequence as set forth in SEQ ID NO: 29 or SEQ ID NO: 30, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein at least one ATG sequence selected from the group (a)-(g) is mutated:
a) ATG corresponding to positions 53-55 of SEQ ID NO: 29;
b) ATG corresponding to positions 72-74 of SEQ ID NO: 29;
c) ATG corresponding to positions 91-93 of SEQ ID NO: 29;
d) ATG corresponding to positions 104-106 of SEQ ID NO: 29;
e) ATG corresponding to positions 121-123 of SEQ ID NO: 29;
f) ATG corresponding to positions 170-172 of SEQ ID NO: 29; and/or
g) ATG corresponding to positions 411-413 of SEQ ID NO: 29.

The WRPE typically contains a retained Pol ORF. An example retained Pol ORF sequence is as follows:

(SEQ ID NO: 31)
ATGCTATTGCTTCCCGTATGGCTTTCATTTTCTCCTCCTTGTATAAATCC
TGGTTGCTGTCTCTTTATGAGGAGTTGTGGCCCGTTGTCAGGCAACGTGG
CGTGGTGTGCACTGTGTTTGCTGACGCAACCCCCACTGGTTGGGGCATTG
CCACCACCTGTCAGCTCCTTTCCGGGACTTTCGCTTTCCCCCTCCCTATT
GCCACGGCGGAACTCATCGCCGCCTGCCTTGCCCGCTGCTGGACAGGGGC
TCGGCTGTTGGGCACTGACAATTCCGTGGTGTTGTCGGGGAAATCATCGT
CCTTTCCTTGGCTGCTCGCCTGTGTTGCCACCTGGATTCTGCGCGGGACG
TCCTTCTGCTACGTCCCTTCGGCCCTCAATCCAGCGGACCTTCCTTCCCG
CGGCCTGCTGCCGGCTCTGCGGCCTCTTCCGCGTCTTCGCCTTCGCCCTC
AGACGAGTCGGATCTCCCTTTGGGCCGCCTCC.

In one embodiment, at least one (suitably at least two or at least three) ATG sequence within the retained Pol ORF sequence in the WPRE is mutated. In one embodiment, all ATG sequences within the retained Pol ORF sequence in the WPRE are mutated.

In one embodiment, the modified WPRE comprises less than three (suitably less than two or less than one) ATG sequences in the retained Pol ORF sequence in the WPRE. In one embodiment, the modified WPRE lacks an ATG sequence in the retained Pol ORF sequence in the WPRE.

An example modified WPRE sequence in which all ATG codons within the retained Pol ORF are mutated is as follows:

(SEQ ID NO: 32)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATTGCTATTGCTTCCCGTATGGCTTTCATTTTCTCCTCCTTGTATAA
ATCCTGGTTGCTGTCTCTTTATTGAGGAGTTGTGGCCCGTTGTCAGGCAA
CGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACA
GGGGCTCGGCTGTTGGGCACTGACAATTCCGTGGTGTTGTCGGGGAAATC
ATCGTCCTTTCCTTGGCTGCTCGCCTGTGTTGCCACCTGGATTCTGCGCG
GGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCG
CCCTCAGACGAGTCGGATCTCCCTTTGGGCCGCCTCC.

An example of a modified WPRE sequence lacking an ATG sequence is as follows:

(SEQ ID NO: 33)
AATCAACCTCTGGATTACAAAATTTGTGAAAGATTGACTGGTATTCTTAA
CTATtGTTGCTCCTTTTACGCTATtGTGGATACGCTGCTTTAATtGCCTT
TGTATCATtGCTATTGCTTCCCGTATtGGCTTTCATTTTCTCCTCCTTGT
ATAAATCCTGGTTGCTGTCTCTTTATtGAGGAGTTGTGGCCCGTTGTCAG
GCAACGTGGCGTGGTGTGCACTGTGTTTGCTGACGCAACCCCCACTGGTT
GGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTG
GACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTG
CGCGGGACGTCCTTCTGCTACGTCCCTTCGGCCCTCAATCCAGCGGACCT
TCCTTCCCGCGGCCTGCTGCCGGCTCTGCGGCCTCTTCCGCGTCTTCGCC
TTCGCCCTCAGACGAGTCGGATCTCCCTTTGGGCCGCCTCC.

The modified WPRE may comprise the sequence as set forth in SEQ ID NO: 32 or SEQ ID NO: 33, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than six (suitably less than five, less than four, less than three, less than two or less than one) ATG sequences.

### Vector/Expression Cassette

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into and expressed by a target cell. The vector may facilitate the integration of the nucleotide sequence encoding a viral vector component to maintain the nucleotide sequence encoding the viral vector component and its expression within the target cell.

The vector may be or may include an expression cassette (also termed an expression construct). Expression cassettes as described herein comprise regions of nucleic acid containing sequences capable of being transcribed. Thus, sequences encoding mRNA, tRNA and rRNA are included within this definition.

The vector may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell. The vector may further comprise a nucleotide sequence enabling the vector to replicate in the host cell in question, such as a conditionally replicating oncolytic vector.

The term “cassette”-which is synonymous with terms such as “conjugate”, “construct” and “hybrid”-includes a polynucleotide sequence directly or indirectly attached to a promoter. Preferably the cassette comprises at least a polynucleotide sequence operably linked to a promoter. For example, expression cassettes for use in the invention may comprise a promoter for the expression of the nucleotide sequence encoding a viral vector component and optionally a regulator of the nucleotide sequence encoding the viral vector component.

The choice of expression cassette, e.g. plasmid, cosmid, virus or phage vector, will often depend on the host cell into which it is to be introduced. The expression cassette can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

### Lentiviral Vectors

Lentiviruses are part of a larger group of retroviruses. A detailed list of lentiviruses may be found in Coffin et al (1997) “Retroviruses” Cold Spring Harbour Laboratory Press Eds: J M Coffin, SM Hughes, HE Varmus pp 758-763). In brief, lentiviruses can be divided into primate and non-primate groups. Examples of primate lentiviruses include but are not limited to: the human immunodeficiency virus (HIV), the causative agent of human acquired-immunodeficiency syndrome (AIDS), and the simian immunodeficiency virus (SIV). The non-primate lentiviral group includes the prototype “slow virus” Visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). In one embodiment, the lentiviral vector is derived from HIV-1, HIV-2, SIV, FIV, BIV, EIAV, CAEV or Visna lentivirus.

The lentivirus family differs from retroviruses in that lentiviruses have the capability to infect both dividing and non-dividing cells (Lewis et al (1992) EMBO J 11 (8): 3053-3058 and Lewis and Emerman (1994) J Virol 68 (1): 510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells and expresses a nucleotide of interest (NOI), or nucleotides of interest.

The lentiviral vector may be used to replicate the NOI in a compatible target cell in vitro. Thus, described herein is a method of making proteins in vitro by introducing a vector of the invention into a compatible target cell in vitro and growing the target cell under conditions which result in expression of the NOI. Protein and NOI may be recovered from the target cell by methods well known in the art. Suitable target cells include mammalian cell lines and other eukaryotic cell lines.

In some embodiment, the vectors may have “insulators”-genetic sequences that block the interaction between promoters and enhancers, and act as a barrier reducing read-through from an adjacent gene.

In one embodiment, the insulator is present between one or more of the lentiviral nucleic acid sequences to prevent promoter interference and read-thorough from adjacent genes. If the insulators are present in the vector between one or more of the lentiviral nucleic acid sequences, then each of these insulated genes may be arranged as individual expression units.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components-these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical retroviral vector as described herein, at least part of one or more protein coding regions essential for replication may be removed from the virus; for example, gag/pol and env may be absent or not functional. This makes the viral vector replication-defective.

The lentiviral vector may be derived from either a primate lentivirus (e.g. HIV-1) or a non-primate lentivirus (e.g. EIAV).

In general terms, a typical retroviral vector production system involves the separation of the viral genome from the essential viral packaging functions. These viral vector components are normally provided to the production cells on separate DNA expression cassettes (alternatively known as plasmids, expression plasmids, DNA constructs or expression constructs).

The vector genome comprises the NOI. Vector genomes typically require a packaging signal (w), the internal expression cassette harbouring the NOI, (optionally) a post-transcriptional element (PRE), typically a central polypurine tract (cppt), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665, which allows for vector production in the absence of rev.

The packaging functions include the gag/pol and env genes. These are required for the production of vector particles by the production cell. Providing these functions in trans to the genome facilitates the production of replication-defective viral vectors.

Production systems for gamma-retroviral vectors are typically 3-component systems requiring genome, gag/pol and env expression constructs. Production systems for HIV-1-based lentiviral vectors may additionally require the accessory gene rev to be provided and for the vector genome to include the rev-responsive element (RRE). EIAV-based lentiviral vectors do not require rev to be provided in trans if an open-reading frame (ORF) is present within the genome (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1a, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human α1-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAlb promoter, SV40/CD43, SV40/CD45, NSE/RU5′ promoter, ICAM-2 promoter, GPllb promoter, GFAP promoter, Fibronectin promoter, Endoglin promoter, Elastase-1 promoter, Desmin promoter, CD68 promoter, CD14 promoter and B29 promoter may be used to drive transcription.

Production of retroviral vectors involves either the transient co-transfection of the production cells with these DNA components or use of stable production cell lines wherein all the components are stably integrated within the production cell genome (e.g. Stewart H J, Fong-Wong L, Strickland I, Chipchase D, Kelleher M, Stevenson L, Thoree V, McCarthy J, Ralph G S, Mitrophanous K A and Radcliffe P A. (2011). Hum Gene Ther. March; 22 (3): 357-69). An alternative approach is to use a stable packaging cell (into which the packaging components are stably integrated) and then transiently transfect in the vector genome plasmid as required (e.g. Stewart, H. J., M. A. Leroux-Carlucci, C. J. Sion, K. A. Mitrophanous and P. A. Radcliffe (2009). Gene Ther. June; 16 (6):805-14). It is also feasible that alternative, not complete, packaging cell lines could be generated (just one or two packaging components are stably integrated into the cell lines) and to generate vector the missing components are transiently transfected. The production cell may also express regulatory proteins such as a member of the tet repressor (TetR) protein group of transcription regulators (e.g. T-Rex, Tet-On, and Tet-Off), a member of the cumate inducible switch system group of transcription regulators (e.g. cumate repressor (CymR) protein), or an RNA-binding protein (e.g. TRAP-tryptophan-activated RNA-binding protein).

In one embodiment of the present invention, the viral vector is derived from EIAV. EIAV has the simplest genomic structure of the lentiviruses and is particularly preferred for use in the present invention. In addition to the gag/pol and env genes, EIAV encodes three other genes: tat, rev, and S2. Tat acts as a transcriptional activator of the viral LTR (Derse and Newbold (1993) Virology 194 (2): 530-536 and Maury et al (1994) Virology 200 (2): 632-642) and rev regulates and coordinates the expression of viral genes through rev-response elements (RRE) (Martarano et al. (1994) J Virol 68 (5): 3102-3111). The mechanisms of action of these two proteins are thought to be broadly similar to the analogous mechanisms in the primate viruses (Martarano et al. (1994) J Virol 68 (5): 3102-3111). The function of S2 is unknown. In addition, an ElAV protein, Ttm, has been identified that is encoded by the first exon of tat spliced to the env coding sequence at the start of the transmembrane protein. In an alternative embodiment of the present invention the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of transducing a target cell. Transduction of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector of the present invention has a minimal viral genome.

As used herein, the term “minimal viral genome” means that the viral vector has been manipulated so as to remove the non-essential elements whilst retaining the elements essential to provide the required functionality to infect, transduce and deliver a NOI to a target cell. Further details of this strategy can be found in WO 1998/17815 and WO 99/32646. A minimal ElAV vector lacks tat, rev and S2 genes and neither are these genes provided in trans in the production system. A minimal HIV vector lacks vif, vpr, vpu, tat and nef.

The expression plasmid used to produce the vector genome within a production cell may include transcriptional regulatory control sequences operably linked to the retroviral genome to direct transcription of the genome in a production cell/packaging cell. All 3rd generation lentiviral vectors are deleted in the 5′ U3 enhancer-promoter region, and transcription of the vector genome RNA is driven by heterologous promoter such as another viral promoter, for example the CMV promoter, as discussed below. This feature enables vector production independently of tat. Some lentiviral vector genomes require additional sequences for efficient virus production. For example, particularly in the case of HIV, RRE sequences may be included. However the requirement for RRE on the (separate) GagPol cassette (and dependence on rev which is provided in trans) may be reduced or eliminated by codon optimisation of the GagPol ORF. Further details of this strategy can be found in WO 2001/79518.

Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents of the Rev protein which are known or become available may be relevant to the invention. For example, it is also known that the Rex protein of HTLV-I can functionally replace the Rev protein of HIV-1. Rev and RRE may be absent or non-functional in the vector for use in the methods of the present invention; in the alternative rev and RRE, or functionally equivalent system, may be present.

It is therefore understood that ‘rev’ may refer to a sequence encoding the HIV-1 Rev protein or a sequence encoding any functional equivalent thereof. Thus, the invention provides a viral vector production system and/or a cell comprising a set of nucleotide sequences, wherein the nucleotide sequences encode vector components including gag-pol, env, optionally rev, and the nucleotide sequences of the invention.

As used herein, the term “functional substitute” means a protein or sequence having an alternative sequence which performs the same function as another protein or sequence. The term “functional substitute” is used interchangeably with “functional equivalent” and “functional analogue” herein with the same meaning.

### SIN Vectors

The lentiviral vectors as described herein may be used in a self-inactivating (SIN) configuration in which the viral enhancer and promoter sequences have been deleted. SIN vectors can be generated and transduce non-dividing target cells in vivo, ex vivo or in vitro with an efficacy similar to that of non-SIN vectors. The transcriptional inactivation of the long terminal repeat (LTR) in the SIN provirus should prevent mobilisation of vRNA, and is a feature that further diminishes the likelihood of formation of replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is important to prevent the adventitious activation of any endogenous oncogene. Yu et al., (1986) PNAS 83:3194-98; Marty et al., (1990) Biochimie 72:885-7; Naviaux et al., (1996) J. Virol. 70:5701-5; Iwakuma et al., (1999) Virol. 261:120-32; Deglon et al., (2000) Human Gene Therapy 11:179-90. SIN lentiviral vectors are described in U.S. Pat. Nos. 6,924,123 and 7,056,699.

### Replication-Defective Lentiviral Vectors

In the genome of a replication-defective lentiviral vector the sequences of gag/pol and/or env may be mutated and/or not functional.

In a typical lentiviral vector as described herein, at least part of one or more coding regions for proteins essential for virus replication may be removed from the vector. This makes the viral vector replication-defective. Portions of the viral genome may also be replaced by a NOI in order to generate a vector comprising an NOI which is capable of transducing a non-dividing target cell and/or integrating its genome into the target cell genome.

In one embodiment the lentiviral vectors are non-integrating vectors as described in WO 2006/010834 and WO 2007/071994.

In a further embodiment the vectors have the ability to deliver a sequence which is devoid of or lacking viral RNA. In a further embodiment a heterologous binding domain (heterologous to gag) located on the RNA to be delivered and a cognate binding domain on Gag or GagPol can be used to ensure packaging of the RNA to be delivered. Both of these vectors are described in WO 2007/072056.

### NOI and Polynucleotides

Polynucleotides of the invention may comprise DNA or RNA. They may be single-stranded or double-stranded. A nucleotide, or nucleotides, of interest is/are commonly referred to as NOI. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing PCR under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Common Retroviral Vector Elements

### Promoters and Enhancers

Expression of a NOI and polynucleotide may be controlled using control sequences for example transcription regulation elements or translation repression elements, which include promoters, enhancers and other expression regulation signals (e.g. tet repressor (TetR) system) or the Transgene Repression In vector Production cell system (TRIP) or other regulators of NOIs described herein.

Prokaryotic promoters and promoters functional in eukaryotic cells may be used. Tissue-specific or stimuli-specific promoters may be used. Chimeric promoters may also be used comprising sequence elements from two or more different promoters.

Suitable promoting sequences are strong promoters including those derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters, such as the actin promoter, EF1a, CAG, TK, SV40, ubiquitin, PGK or ribosomal protein promoter. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2),

Tyrosine hydroxylase, neuronal-specific neuronal-specific enolase (NSE) promoter, astrocyte-specific glial fibrillary acidic protein (GFAP) promoter, human α1-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAlb promoter, SV40/CD43, SV40/CD45, NSE/RU5′ promoter, ICAM-2 promoter, GPllb promoter, GFAP promoter, Fibronectin promoter, Endoglin promoter, Elastase-1 promoter, Desmin promoter, CD68 promoter, CD14 promoter and B29 promoter may be used to drive transcription.

Transcription of a NOI may be increased further by inserting an enhancer sequence into the vector. Enhancers are relatively orientation- and position-independent; however, one may employ an enhancer from a eukaryotic cell virus, such as the SV40 enhancer and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable target cell. For example, the features can be conserved regions e.g. a Pribnow Box or a TATA box. The promoter may contain other sequences to affect (such as to maintain, enhance or decrease) the levels of expression of a nucleotide sequence. Suitable other sequences include the Sh1-intron or an ADH intron. Other sequences include inducible elements, such as temperature, chemical, light or stress inducible elements. Also, suitable elements to enhance transcription or translation may be present.

### Regulators of NOIs

A complicating factor in the generation of retroviral packaging/producer cell lines and retroviral vector production is that constitutive expression of certain retroviral vector components and NOIs are cytotoxic leading to death of cells expressing these components and therefore inability to produce vector. Therefore, the expression of these components (e.g. gag-pol and envelope proteins such as VSV-G) can be regulated. The expression of other non-cytotoxic vector components, e.g. rev, can also be regulated to minimise the metabolic burden on the cell. The modular constructs and/or cells as described herein may comprise cytotoxic and/or non-cytotoxic vector components associated with at least one regulatory element. As used herein, the term “regulatory element” refers to any element capable of affecting, either increasing or decreasing, the expression of an associated gene or protein. A regulatory element includes a gene switch system, transcription regulation element and translation repression element.

A number of prokaryotic regulator systems have been adapted to generate gene switches in mammalian cells. Many retroviral packaging and producer cell lines have been controlled using gene switch systems (e.g. tetracycline and cumate inducible switch systems) thus enabling expression of one or more of the retroviral vector components to be switched on at the time of vector production. Gene switch systems include those of the (TetR) protein group of transcription regulators (e.g.T-Rex, Tet-On, and Tet-Off), those of the cumate inducible switch system group of transcription regulators (e.g. CymR protein) and those involving an RNA-binding protein (e.g. TRAP).

One such tetracycline-inducible system is the tetracycline repressor (TetR) system based on the T-REx™ system. By way of example, in such a system tetracycline operators (TetO2) are placed in a position such that the first nucleotide is 10 bp from the 3′ end of the last nucleotide of the TATATAA element of the human cytomegalovirus major immediate early promoter (hCMVp) then TetR alone is capable of acting as a repressor (Yao F, Svensjo T, Winkler T, Lu M, Eriksson C, Eriksson E. Tetracycline repressor, tetR, rather than the tetR-mammalian cell transcription factor fusion derivatives, regulates inducible gene expression in mammalian cells. 1998. Hum Gene Ther, 9:1939-1950). In such a system the expression of the NOI can be controlled by a CMV promoter into which two copies of the TetO2 sequence have been inserted in tandem. TetR homodimers, in the absence of an inducing agent (tetracycline or its analogue doxycycline [dox]), bind to the TetO2 sequences and physically block transcription from the upstream CMV promoter. When present, the inducing agent binds to the TetR homodimers, causing allosteric changes such that it can no longer bind to the TetO2 sequences, resulting in gene expression. The TetR gene may be codon optimised as this may improve translation efficiency resulting in tighter control of TetO2 controlled gene expression.

The TRIP system is described in WO 2015/092440 and provides another way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) March 27; 8).

Briefly, the TRAP-tbs interaction forms a translational block, repressing translation of the transgene protein (Maunder et al, Nat Commun. (2017) March 27; 8). The translational block is only effective in production cells and as such does not impede the DNA-or RNA-based vector systems. The TRIP system is able to repress translation when the transgene protein is expressed from a constitutive and/or strong promoter, including a tissue-specific promoter from single- or multi cistronic mRNA. It has been demonstrated that unregulated expression of transgene protein can reduce vector titres and affect vector product quality. Repression of transgene protein for both transient and stable PaCL/PCL vector production systems is beneficial for production cells to prevent a reduction in vector titres: where toxicity or molecular burden issues may lead to cellular stress; where transgene protein elicits an immune response in vivo due to viral vector delivery of transgene-derived protein; where the use of gene-editing transgenes may result in on/off target affects; where the transgene protein may affect vector and/or envelope glycoprotein exclusion.

### Other Envelopes and Pseudotyping

Any suitable envelope may be used in accordance with the present invention. Whilst Nipah envelopes, as described herein, are preferred and exemplified, other envelopes can be utilised to form mixed envelope vectors as described herein.

A non-exhaustive set of example envelopes are described below.

### VSV-G

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207. WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72 (8) 6356-6361 teach that non-infectious retroviral particles can be made infectious by the addition of VSV-G.

Burns et al. (1993) Proc. Natl. Acad. Sci. USA 90:8033-7 successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages for both efficient target cell infection/transduction and during manufacturing processes.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

### Ross River Virus

The Ross River viral envelope has been used to pseudotype a non-primate lentiviral vector (FIV) and following systemic administration predominantly transduced the liver (Kang et al., 2002, J. Virol., 76:9378-9388). Efficiency was reported to be 20-fold greater than obtained with VSV-G pseudotyped vector, and caused less cytotoxicity as measured by serum levels of liver enzymes suggestive of hepatotoxicity.

### Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1): 67-77). Compared with VSV-G-pseudotyped vectors, GP64-pseudotyped vectors have a similar broad tropism and similar native titres. Because, GP64 expression does not kill cells, HEK293T-based cell lines constitutively expressing GP64 can be generated.

### Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

### Packaging Sequence

As utilized within the context of the present invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon (some or all of the 5′ sequence of gag to nucleotide 688 may be included). In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag (Kaye et al., J Virol. October;69(10):6588-92 (1995).

### Internal Ribosome Entry Site (IRES)

Insertion of IRES elements allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44:292-309). When located between open reading frames in an RNA, IRES elements allow efficient translation of the downstream open reading frame by promoting entry of the ribosome at the IRES element followed by downstream initiation of translation.

A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334:320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES. The IRES(s) may be of viral origin (such as EMCV IRES, PV IRES, or FMDV 2A-like sequences) or cellular origin (such as FGF2 IRES, NRF IRES, Notch 2 IRES or EIF4 IRES).

In order for the IRES to be capable of initiating translation of each polynucleotide it should be located between or prior to the polynucleotides in the modular construct.

The nucleotide sequences utilised for development of stable cell lines require the addition of selectable markers for selection of cells where stable integration has occurred. These selectable markers can be expressed as a single transcription unit within the nucleotide sequence or it may be preferable to use IRES elements to initiate translation of the selectable marker in a polycistronic message (Adam et al 1991 J.Virol. 65, 4985).

### Genetic Orientation and Insulators

It is well known that nucleic acids are directional and this ultimately affects mechanisms such as transcription and replication in the cell. Thus genes can have relative orientations with respect to one another when part of the same nucleic acid construct.

In certain embodiments of the present invention, at least two nucleic acid sequences present at the same locus in the cell or construct can be in a reverse and/or alternating orientations. In other words, in certain embodiments of the invention at this particular locus, the pair of sequential genes will not have the same orientation. This can help prevent both transcriptional and translational read-through when the region is expressed within the same physical location of the host cell.

Having the alternating orientations benefits retroviral vector production when the nucleic acids required for vector production are based at the same genetic locus within the cell. This in turn can also improve the safety of the resulting constructs in preventing the generation of replication-competent retroviral vectors.

When nucleic acid sequences are in reverse and/or alternating orientations the use of insulators can prevent inappropriate expression or silencing of a NOI from its genetic surroundings.

The term “insulator” refers to a class of nucleotide, e.g. DNA, sequence elements that when bound to insulator-binding proteins possess an ability to protect genes from surrounding regulator signals. There are two types of insulators: an enhancer blocking function and a chromatin barrier function. When an insulator is situated between a promoter and an enhancer, the enhancer-blocking function of the insulator shields the promoter from the transcription-enhancing influence of the enhancer (Geyer and Corces 1992; Kellum and Schedl 1992). The chromatin barrier insulators function by preventing the advance of nearby condensed chromatin which would lead to a transcriptionally active chromatin region turning into a transcriptionally inactive chromatin region and resulting in silencing of gene expression. Insulators which inhibit the spread of heterochromatin, and thus gene silencing, recruit enzymes involved in histone modifications to prevent this process (Yang J, Corces V G. 2011; 110:43-76; Huang, Li et al. 2007; Dhillon, Raab et al. 2009). An insulator can have one or both of these functions and the chicken β-globin insulator (cHS4) is one such example. This insulator is the most extensively studied vertebrate insulator, is highly rich in G+C and has both enhancer-blocking and heterochromatic barrier functions (Chung J H, Whitely M, Felsenfeld G. Cell. 1993; 74:505-514). Other such insulators with enhancer blocking functions are not limited to but include the following: human β-globin insulator 5 (HS5), human β-globin insulator 1 (HS1), and chicken β-globin insulator (cHS3) (Farrell CM1, West A G, Felsenfeld G., Mol Cell Biol. 2002 June;22 (11): 3820-31; J Ellis et al. EMBO J. 1996 Feb. 1; 15 (3): 562-568). In addition to reducing unwanted distal interactions the insulators also help to prevent promoter interference (i.e. where the promoter from one transcription unit impairs expression of an adjacent transcription unit) between adjacent retroviral nucleic acid sequences. If the insulators are used between each of the retroviral vector nucleic acid sequences, then the reduction of direct read-through will help prevent the formation of replication-competent retroviral vector particles.

The insulator may be present between each of the retroviral nucleic acid sequences. In one embodiment, the use of insulators prevents promoter-enhancer interactions from one NOI expression cassette interacting with another NOI expression cassette in a nucleotide sequence encoding vector components.

An insulator may be present between the vector genome and gag-pol sequences. This therefore limits the likelihood of the production of a replication-competent retroviral vector and ‘wild-type’ like RNA transcripts, improving the safety profile of the construct. The use of insulator elements to improve the expression of stably integrated multigene vectors is cited in Moriarity et al, Nucleic Acids Res. 2013 April;41 (8): e92.

### Vector Titre

The skilled person will understand that there are a number of different methods of determining the titre of lentiviral vectors. Titre is often described as transducing units/mL (TU/mL). Titre may be increased by increasing the number of vector particles and by increasing the specific activity of a vector preparation.

### Therapeutic Use

The lentiviral vector production system or lentiviral vector as described herein or a cell or tissue transduced with the lentiviral vector as described herein may be used in medicine.

In addition, the lentiviral vector as described herein, a production cell of the invention or a cell or tissue transduced with the lentiviral vector as described herein may be used for the preparation of a medicament to deliver a nucleotide of interest to a target site in need of the same. Such uses of the lentiviral vector or transduced cell of the invention may be for therapeutic or diagnostic purposes, as described previously.

Accordingly, there is provided a cell transduced by the lentiviral vector as described herein.

A “cell transduced by a viral vector particle” is to be understood as a cell, in particular a target cell, into which the nucleic acid carried by the viral vector particle has been transferred.

### Nucleotide of Interest

In one embodiment of the invention, the nucleotide of interest (i.e. transgene) is translated in a target cell which lacks TRAP.

“Target cell” is to be understood as a cell in which it is desired to express the NOI. The NOI may be introduced into the target cell using a viral vector of the present invention. Delivery to the target cell may be performed in vivo, ex vivo or in vitro.

In a preferred embodiment, the nucleotide of interest gives rise to a therapeutic effect.

The NOI may have a therapeutic or diagnostic application. Suitable NOIs include, but are not limited to sequences encoding enzymes, co-factors, cytokines, chemokines, hormones, antibodies, anti-oxidant molecules, engineered immunoglobulin-like molecules, single chain antibodies, fusion proteins, immune co-stimulatory molecules, immunomodulatory molecules, chimeric antigen receptors a transdomain negative mutant of a target protein, toxins, conditional toxins, antigens, transcription factors, structural proteins, reporter proteins, subcellular localization signals, tumour suppressor proteins, growth factors, membrane proteins, receptors, vasoactive proteins and peptides, anti-viral proteins and ribozymes, and derivatives thereof (such as derivatives with an associated reporter group). The NOIs may also encode micro-RNA . . .

In one embodiment, the NOI may be useful in the treatment of a neurodegenerative disorder.

In another embodiment, the NOI may be useful in the treatment of Parkinson's disease and/or multiple system atrophy.

In another embodiment, the NOI may encode an enzyme or enzymes involved in dopamine synthesis. For example, the enzyme may be one or more of the following: tyrosine hydroxylase, GTP-cyclohydrolase I and/or aromatic amino acid dopa decarboxylase. The sequences of all three genes are available (GenBank® Accession Nos. X05290, U19523 and M76180, respectively).

In another embodiment, the NOI may encode the vesicular monoamine transporter 2 (VMAT2). In an alternative embodiment, the viral genome may comprise a NOI encoding aromatic amino acid dopa decarboxylase and a NOI encoding VMAT2. Such a genome may be used in the treatment of Parkinson's disease, in particular in conjunction with peripheral administration of L-DOPA.

In another embodiment, the NOI may encode a therapeutic protein or combination of therapeutic proteins.

In another embodiment, the NOI may encode a protein or proteins selected from the group consisting of glial cell derived neurotrophic factor (GDNF), brain derived neurotrophic factor (BDNF), ciliary neurotrophic factor (CNTF), neurotrophin-3 (NT-3), acidic fibroblast growth factor (aFGF), basic fibroblast growth factor (bFGF), interleukin-1 beta (IL-1β), tumor necrosis factor alpha (TNF-α), insulin growth factor-2, VEGF-A, VEGF-B, VEGF-C/VEGF-2, VEGF-D, VEGF-E, PDGF-A, PDGF-B, hetero- and homo-dimers of PDFG-A and PDFG-B.

In another embodiment, the NOI may encode an anti-angiogenic protein or anti-angiogenic proteins selected from the group consisting of angiostatin, endostatin, platelet factor 4, pigment epithelium derived factor (PEDF), placental growth factor, restin, interferon-α, interferon-inducible protein, gro-beta and tubedown-1, interleukin (IL)-1, IL-12, retinoic acid, anti-VEGF antibodies or fragments/variants thereof such as aflibercept, thrombospondin, VEGF receptor proteins such as those described in U.S. Pat. Nos. 5,952,199 and 6,100,071, and anti-VEGF receptor antibodies.

In another embodiment, the NOI may encode anti-inflammatory proteins, antibodies or fragment/variants of proteins or antibodies selected from the group consisting of NF-kB inhibitors, IL1beta inhibitors, TGFbeta inhibitors, IL-6 inhibitors, IL-23 inhibitors, IL-18 inhibitors, Tumour necrosis factor alpha and Tumour necrosis factor beta, Lymphotoxin alpha and Lymphotoxin beta, LIGHT inhibitors, alpha synuclein inhibitors, Tau inhibitors, beta amyloid inhibitors, IL-17 inhibitors, IL-33 inhibitors, IL-33 receptor inhibitors and TSLP inhibitors.

In another embodiment the NOI may encode cystic fibrosis transmembrane conductance regulator (CFTR).

In another embodiment the NOI may encode a protein normally expressed in an ocular cell.

In another embodiment, the NOI may encode a protein normally expressed in a photoreceptor cell and/or retinal pigment epithelium cell.

In another embodiment, the NOI may encode a protein selected from the group comprising RPE65, arylhydrocarbon-interacting receptor protein like 1 (AIPL1), CRB1, lecithin retinal acetyltransferace (LRAT), photoreceptor-specific homeo box (CRX), retinal guanylate cyclise (GUCY2D), RPGR interacting protein 1 (RPGRIP1), LCA2, LCA3, LCA5, dystrophin, PRPH2, CNTF, ABCR/ABCA4, EMP1, TIMP3, MERTK, ELOVL4, MYO7A, USH2A, VMD2, RLBP1, COX-2, FPR, harmonin, Rab escort protein 1, CNGB2, CNGA3, CEP 290, RPGR, RS1, RP1, PRELP, glutathione pathway enzymes and opticin.

In other embodiments, the NOI may encode the human clotting Factor VIII or Factor IX.

In other embodiments, the NOI may encode protein or proteins involved in metabolism selected from the group comprising phenylalanine hydroxylase (PAH), Methylmalonyl CoA mutase, Propionyl CoA carboxylase, Isovaleryl CoA dehydrogenase, Branched chain ketoacid dehydrogenase complex, Glutaryl CoA dehydrogenase, Acetyl CoA carboxylase, propionyl CoA carboxylase, 3 methyl crotonyl CoA carboxylase, pyruvate carboxylase, carbamoyl-phophate synthase ammonia, ornithine transcarbamylase, glucosylceramidase beta, alpha galactosidase A, glucosylceramidase beta, cystinosin, glucosamine (N-acetyl)-6-sulfatase, N-acetyl-alpha-glucosaminidase, glucose-6-phosphatase, ATP7B, ATP8B1, ABCB11, ABCB4, TJP2, N-sulfoglucosamine sulfohydrolase, Galactosamine-6 sulfatase, arylsulfatase A, cytochrome B-245 beta, ABCD1, ornithine carbamoyltransferase, argininosuccinate synthase, argininosuccinate lysase, arginase 1, alanine glycoxhylate amino transferase, ATP-binding cassette, sub-family B members.

In other embodiments, the NOI may encode a chimeric antigen receptor (CAR) or a T cell receptor (TCR). In one embodiment, the CAR is an anti-5T4 CAR. In other embodiments, the NOI may encode B-cell maturation antigen (BCMA), CD19, CD22, CD20, CD138, CD30, CD33, CD123, CD70, prostate specific membrane antigen (PSMA), Lewis Y antigen (LeY), Tyrosine-protein kinase transmembrane receptor (ROR1), Mucin 1, cell surface associated (Muc1), Epithelial cell adhesion molecule (EpCAM), endothelial growth factor receptor (EGFR), insulin, protein tyrosine phosphatase, non-receptor type 22, interleukin 2 receptor, alpha, interferon induced with helicase C domain 1, human epidermal growth factor receptor (HER2), glypican 3 (GPC3), disialoganglioside (GD2), mesiothelin, vesicular endothelial growth factor receptor 2 (VEGFR2), Smith antigen, Ro60, double stranded DNA, phospholipids, proinsulin, insulinoma antigen 2 (IA-2), 65 kDa isoform of glutamic acid decarboxylase (GAD65), chromogranin A (CHGA), islet amyloid polypeptide (IAPP), islet-specific glucose-6-phosphatase catalytic subunit-related protein (IGRP), zinc transporter 8 (ZnT8).

In other embodiments, the NOI may encode a chimeric antigen receptor (CAR) against NKG2D ligands selected from the group comprising ULBP1, 2 and 3, H60, Rae-1a, b, g, d, MICA, MICB.

In further embodiments the NOI may encode SGSH, SUMF1, GAA, the common gamma chain (CD132), adenosine deaminase, WAS protein, globins, alpha galactosidase A, δ-aminolevulinate (ALA) δ-aminolevulinate synthase, dehydratase (ALAD), Hydroxymethylbilane (HMB) synthase, Uroporphyrinogen (URO) synthase, Uroporphyrinogen (URO) decarboxylase, Coproporphyrinogen (COPRO) oxidase, Protoporphyrinogen (PROTO) oxidase, Ferrochelatase, α-L-iduronidase, Iduronate sulfatase, Heparan sulfamidase, N-acetylglucosaminidase, Heparan-α-glucosaminide N-acetyltransferase, 3 N-acetylglucosamine 6-sulfatase, Galactose-6-sulfate sulfatase, β-galactosidase, N-acetylgalactosamine-4-sulfatase, β-glucuronidase and Hyaluronidase.

In addition to the NOI the vector may also comprise or encode a siRNA, shRNA, or regulated shRNA (Dickins et al. (2005) Nature Genetics 37:1289-1295, Silva et al. (2005) Nature Genetics 37:1281-1288).

### Indications

The vectors, including retroviral and AAV vectors, according to the present invention may be used to deliver one or more NOI(s) useful in the treatment of the disorders listed in WO 1998/05635, WO 1998/07859, and WO 1998/09985. The nucleotide of interest may be DNA or RNA. Examples of such diseases are given below:

A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells.

Malignancy disorders, including cancer, leukaemia, benign and malignant tumour growth, invasion and spread, angiogenesis, metastases, ascites and malignant pleural effusion.

Autoimmune diseases including arthritis, including rheumatoid arthritis, hypersensitivity, psoriasis, Sjogren's syndrome, allergic reactions, asthma, chronic obstructive pulmonary disease, systemic lupus erythematosus, Type 1 diabetes mellitus, Crohn's disease, ulcerative colitis, collagen diseases and other diseases.

Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases.

Diseases of the gastrointestinal tract including peptic ulcer, ulcerative colitis, Crohn's disease and other diseases.

Hepatic diseases including hepatic fibrosis, liver cirrhosis, amyloidosis.

Inherited metabolic disorders including phenylketonuria PKU, Wilson disease, organic acidemias, glycogen storage diseases, urea cycle disorders, cholestasis, and other diseases.

Renal and urologic diseases including thyroiditis or other glandular diseases, glomerulonephritis, lupus nephritis or other diseases.

Ear, nose and throat disorders including otitis or other oto-rhino-laryngological diseases, dermatitis or other dermal diseases.

Dental and oral disorders including periodontal diseases, periodontitis, gingivitis or other dental/oral diseases.

Testicular diseases including orchitis or epididimo-orchitis, infertility, orchidal trauma or other testicular diseases.

Gynaecological diseases including placental dysfunction, placental insufficiency, habitual abortion, eclampsia, pre-eclampsia, endometriosis and other gynaecological diseases.

Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia.

Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wiscott Aldrich Syndrome, adrenoleukodystrophy, beta-thalassemia, sickle cell disease, Guillaim-Barre syndrome, Sydenham chorea, myasthenia gravis, pseudo-tumour cerebri, Down's Syndrome, Huntington's disease, Frontotemporal dementia, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury.

Other diseases and conditions such as cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.

SIRNA, micro-RNA and shRNA

In certain other embodiments, the NOI comprises a micro-RNA. The micro-RNA which is the NOI (i.e. transgene) is distinct from the self-destabilisation or self-decay element described herein. Thus, a micro-RNA which is the transgene typically does not target the mRNA encoding the transgene. A micro-RNA which is the transgene may target the mRNA encoding another transgene (i.e. a second transgene) in order to regulate the second transgene mRNA, for example, as part of a gene switch system.

In addition to the NOI, the vector may also comprise or encode a miRNA, siRNA, shRNA, or regulated shRNA (Dickins et al. (2005) Nature Genetics 37:1289-1295, Silva et al. (2005) Nature Genetics 37:1281-1288). Preferably, the vector may comprise or encode a miRNA or shRNA in addition to the NOI. The additional miRNA, siRNA, shRNA, or regulated shRNA is distinct from the self-destabilisation or self-decay element described herein. Thus, the additional miRNA, siRNA, shRNA, or regulated shRNA does not target the mRNA encoding the transgene.

### Pharmaceutical Compositions

The present disclosure provides a pharmaceutical composition comprising the viral vector production system, viral vector, lentiviral vector as described herein or a cell or tissue transduced with the viral vector as described herein, in combination with a pharmaceutically acceptable carrier, diluent or excipient.

The present disclosure provides a pharmaceutical composition for treating an individual by gene therapy, wherein the composition comprises a therapeutically effective amount of a lentiviral vector. The pharmaceutical composition may be for human or animal usage.

The composition may comprise a pharmaceutically acceptable carrier, diluent, excipient or adjuvant. The choice of pharmaceutical carrier, excipient or diluent can be made with regard to the intended route of administration and standard pharmaceutical practice. The pharmaceutical compositions may comprise, or be in addition to, the carrier, excipient or diluent any suitable binder(s), lubricant(s), suspending agent(s), coating agent(s), solubilising agent(s) and other carrier agents that may aid or increase vector entry into the target site (such as for example a lipid delivery system).

Where appropriate, the composition can be administered by any one or more of inhalation; in the form of a suppository or pessary; topically in the form of a lotion, solution, cream, ointment or dusting powder; by use of a skin patch; orally in the form of tablets containing excipients such as starch or lactose, or in capsules or ovules either alone or in admixture with excipients, or in the form of elixirs, solutions or suspensions containing flavouring or colouring agents; or they can be injected parenterally, for example intracavernosally, intravenously, intramuscularly, intracranially, intraoccularly intraperitoneally, or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

The lentiviral vector as described herein may also be used to transduce target cells or target tissue ex vivo prior to transfer of said target cell or tissue into a patient in need of the same. An example of such cell may be autologous T cells and an example of such tissue may be a donor cornea.

### Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The term “homologue” means an entity having a certain homology with the wild type amino acid sequence and the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 97 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 97%, 98% or 99% identical to the subject sequence. Although homology can also be considered in terms of similarity, in the context of the present invention it is preferred to express homology in terms of sequence identity.

Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (see Ausubel et al. (1999) ibid-Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol Lett (1999) 174(2): 247-50; FEMS Microbiol Lett (1999) 177(1): 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software usually does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the break. The DNA is then expressed in accordance with the invention to make the encoded protein. These methods are only illustrative of the numerous standard techniques known in the art for manipulation of DNA sequences and other known techniques may also be used.

All variants, fragments or homologues of the regulatory protein suitable for use in the cells and/or modular constructs of the invention will retain the ability to bind the cognate binding site of the NOI such that translation of the NOI is repressed or prevented in a viral vector production cell.

All variants fragments or homologues of the binding site will retain the ability to bind the cognate RNA-binding protein, such that translation of the NOI is repressed or prevented in a viral vector production cell.

### Codon Optimisation

The polynucleotides used in the present invention (including the NOI and/or components of the vector production system) may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including retroviruses, use a large number of rare codons and changing these to correspond to commonly used mammalian codons, increases expression of a gene of interest, e.g. a NOI or packaging components in mammalian production cells, can be achieved. Codon usage tables are known in the art for mammalian cells, as well as for a variety of other organisms.

Codon optimisation of viral vector packaging components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vector gag/pol expression cassettes codon optimisation also overcomes the Rev/RRE requirement for export, rendering optimised sequences Rev-independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with some exceptions, for example the sequence encompassing the frameshift site of gag-pol (see below).

The gag-pol gene of lentiviral vectors comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the Gag-Pol proteins. For EIAV the beginning of the overlap has been taken to be nt 1262 (where nucleotide 1 is the A of the gag ATG) and the end of the overlap to be nt 1461. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the Gag-Pol proteins.

In one embodiment, codon optimisation is based on lightly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon-optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at http://hiv-web.lanl.gov. Details of EIAV clones may be found at the National Center for Biotechnology Information (NCBI) database located at http://www.ncbi.nlm.nih.gov.

The strategy for codon-optimised gag-po/sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimising vector genome components. Advantageously, these modifications also lead to the production of a safer system absent of all additional proteins both in the producer and in the transduced cell.

It is to be understood that features disclosed herein may be used in combination with one another. Furthermore, it is to be understood that such features may possess different functionalities by virtue of the nucleotide sequence comprising them.

The practice of the present invention will employ, unless otherwise indicated, conventional techniques of chemistry, molecular biology, microbiology and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, NY; B. Roe, J. Crabtree, and A. Kahn (1996) DNA Isolation and Sequencing: Essential Techniques, John Wiley & Sons; J. M. Polak and James O′D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

### Aspects of the Invention

The invention may further be described by the following numbered aspects:

Aspect 1. A viral vector production system comprising a set of nucleotide sequences, wherein the nucleotide sequences encode heterologous envelope proteins comprising at least one target binding protein and at least one viral envelope protein.

Aspect 2. The viral vector production system according to aspect 1, wherein the target binding protein is a non-viral protein.

Aspect 3. The viral vector production system according to aspect 1 or aspect 2, wherein the target binding protein is a chimeric protein.

Aspect 4. The viral vector production system according to any one of aspects 1 to 3, wherein the viral envelope protein is a fusion protein.

Aspect 5. The viral vector production system according to any one of aspects 1 to 4, wherein the viral envelope protein is receptor-blinded.

Aspect 6. The viral vector production system according to any one of aspects 1 to 5, wherein the viral envelope protein is a negative sense RNA virus fusion protein or modified version thereof.

Aspect 7. The viral vector production system according to any one of aspects 1 to 6, wherein the viral envelope protein is a mononegavirus fusion protein or modified version thereof.

Aspect 8. The viral vector production system according to any one of aspects 1 to 7, wherein the viral envelope protein is a paramyxovirus fusion protein or modified version thereof.

Aspect 9. The viral vector production system according to any one of aspects 1 to 8, wherein the viral envelope protein is a henipavirus fusion protein or modified version thereof.

Aspect 10. The viral vector production system according to any one of aspects 1 to 9, wherein the viral envelope protein is a Nipah virus fusion protein or modified version thereof.

Aspect 11. The viral vector production system according to any one of aspects 1 to 10, wherein the viral envelope protein:
(a) is a Δ22 mutant of the Nipah virus fusion protein; and/or
(b) comprises a sequence of SEQ ID NO: 3 or SEQ ID NO: 4.

Aspect 12. The viral vector production system according to any one of aspects 1 to 7, wherein the viral envelope protein is a rhabdovirus fusion protein or a modified version thereof.

Aspect 13. The viral vector production system according to any one of aspects 1 to 7, and aspect 12, wherein the viral envelope protein is a Vesicular stomatitis virus fusion protein (VSV-G), or a modified version thereof.

Aspect 14. The viral vector production system according to any one of aspects 1 to 7, and aspects 12 and 13, wherein the viral envelope protein:
(a) is a K47Q mutant of VSV-G;
(b) is a R354A mutant of VSV-G; and/or
(c) comprises a sequence of SEQ ID NO: 34 or SEQ ID NO: 35.

Aspect 15. A viral vector production system comprising a set of nucleotide sequences, wherein the nucleotide sequences encode heterologous envelope protein sequences comprising at least one viral attachment protein, wherein the nucleotide sequences separately encode:
(i) a non-retargeted viral attachment protein; and
(ii) a retargeted viral attachment protein.

Aspect 16. The viral vector production system according to aspect 15, wherein the attachment protein is receptor-blinded such that it does not bind to one or more of its endogenous receptors.

Aspect 17. The viral vector production system according to aspect 15 or aspect 16, wherein the non-retargeted attachment protein and retargeted attachment protein are derived from the same virus.

Aspect 18. The viral vector production system according to any one of aspects 15 to 17, wherein the viral attachment protein is a negative sense RNA virus attachment protein, or a modified version thereof.

Aspect 19. The viral vector production system according to any one of aspects 15 to 18, wherein the viral attachment protein is a mononegavirus attachment protein, or a modified version thereof.

Aspect 20. The viral vector production system according any one of aspects 15 to 19, wherein the viral attachment protein is a paramyxovirus attachment protein, or a modified version thereof.

Aspect 21. The viral vector production system according to any one of aspects 15 to 20, wherein the viral attachment protein is a henipavirus attachment protein, or a modified version thereof.

Aspect 22. The viral vector production system according to any one of aspects 15 to 21, wherein the viral attachment protein is a Nipah virus attachment protein, or a modified version thereof.

Aspect 23. The viral vector production system according to any one of aspects 15 to 22, wherein the viral attachment protein:
(a) is a mA34 mutant of the Nipah virus attachment protein; and/or
(b) comprises a sequence of SEQ ID NO: 1 or SEQ ID NO: 2.

Aspect 24. The viral vector production system according to any one of aspects 15 to 19, wherein the viral attachment protein is a rhabdovirus attachment protein, or a modified version thereof.

Aspect 25. The viral vector production system according to any one of aspects 15 to 19, and aspect 24, wherein the viral attachment protein is a Vesicular stomatitis virus (VSV) protein, or a modified version thereof.

Aspect 26. The viral vector production system according to any one of aspects 15 to 19, and aspects 24 and 25, wherein the viral attachment protein:
(a) is a K47Q mutant of VSV-G;
(b) is a R354A mutant of VSV-G; and/or
(c) comprises a sequence of SEQ ID NO: 34 or SEQ ID NO: 35.

Aspect 27. The viral vector production system according to any one of aspects 15 to 26, wherein the retargeted viral attachment protein comprises a retargeting moiety.

Aspect 28. The viral vector production system according to any one of aspects 15 to 27, wherein the retargeting moiety is a protein.

Aspect 29. The viral vector production system according to any one of aspects 15 to 28, wherein the retargeting moiety is a protein selected from the group consisting of: an antibody, an scFV, a nanobody, a DARPin, and a glycoprotein.

Aspect 30. The viral vector production system according to any one of aspects 15 to 29, wherein the retargeting moiety has affinity for CD8 or CD3.

Aspect 31. The viral vector production system according to aspect 30, wherein the retargeting moiety has affinity for CD8.

Aspect 32. The viral vector production system according to aspect 30, wherein the retargeting moiety has affinity for CD3.

Aspect 33. The viral vector production system according to any one of aspects 15 to 32, wherein the retargeted attachment protein is a chimeric protein.

Aspect 34. The viral vector production system according to any one of aspects 15 to 33, wherein the nucleotide sequences encode a viral fusion protein.

Aspect 35. The viral vector production system according to any one of aspects 15 to 34, wherein the viral fusion protein is a negative sense RNA virus fusion protein or modified version thereof.

Aspect 36. The viral vector production system according to any one of aspects 15 to 35, wherein the viral fusion protein is a mononegavirus fusion protein or modified version thereof.

Aspect 37. The viral vector production system according to any one of aspects 15 to 36, wherein the viral fusion protein is a paramyxovirus fusion protein or modified version thereof.

Aspect 38. The viral vector production system according to any one of aspects 15 to 37, wherein the viral fusion protein is a henipavirus fusion protein or modified version thereof.

Aspect 39. The viral vector production system according to any one of aspects 15 to 38, wherein the viral fusion protein is a Nipah virus fusion protein or modified version thereof.

Aspect 40. The viral vector production system according to any one of aspects 15 to 39, wherein the viral fusion protein:
(a) is a Δ22 mutant of the Nipah virus fusion protein; and/or
(b) comprises a sequence of SEQ ID NO: 3 or SEQ ID NO: 4.

Aspect 41. The viral vector production system according to any one of aspects 15 to 36, wherein the viral attachment protein is a rhabdovirus fusion protein, or a modified version thereof.

Aspect 42. The viral vector production system according to any one of aspects 15 to 36, and aspect 41, wherein the viral fusion protein is a Vesicular stomatitis virus (VSV) protein, or a modified version thereof.

Aspect 43. The viral vector production system according to any one of aspects 15 to 36, and aspects 41 and 42, wherein the viral fusion protein:
(a) is a K47Q mutant of VSV-G;
(b) is a R354A mutant of VSV-G; and/or
(c) comprises a sequence of SEQ ID NO: 34 or SEQ ID NO: 35.

Aspect 44. The viral vector production system according to any one of aspects 15 to 43, wherein the ratio of non-retargeted viral attachment protein sequences to retargeted viral attachment protein sequences is within the range of 0:1 to 1:0 but is neither 0:1 or 1:0, such as within the range of from 0.95:0.05 to 0.05:0.95, such as 0.125:0.875, 0.25:0.75, 0.375:0.625, 0.5:0.5, or 0.75:0.25.

Aspect 45. The viral vector production system according to any one of aspects 15 to 44, wherein the ratio of non-retargeted viral attachment protein sequences to retargeted viral attachment protein sequences is 0.125:0.875, 0.25:0.75, 0.375:0.625, 0.5:0.5, or 0.75:0.25.

Aspect 46. A cell comprising the viral vector production system according to any one of aspects 1 to 45.

Aspect 47. A cell for producing viral vectors comprising the viral vector production system according to any one of aspects 1 to 45.

Aspect 48. A method for producing a viral vector, comprising the steps of:
(a) introducing the viral vector production system according to any one of aspects 1 to 45 into a cell;
(b) optionally, selecting for a cell which comprises the nucleotide sequences of the viral vector production system according to any one of aspects 1 to 45; and
(c) culturing the cell under conditions suitable for the production of the viral vector.

Aspect 49. A method for producing a viral vector, comprising the steps of:
(a) introducing the viral vector production system according to any one of aspects 15 to 45 into a cell;
(b) optionally, selecting for a cell which comprises the nucleotide sequences of the viral vector production system according to any one of aspects 15 to 45; and
(c) culturing the cell under conditions suitable for the production of the viral vector.

Aspect 50. A viral vector produced by the method according to aspect 48 or aspect 49.

Aspect 51. The viral vector produced by the method according to aspect 49, wherein the viral vector envelope comprises:
(i) a non-retargeted attachment protein; and
(ii) a retargeted attachment protein; and, optionally
(iii) a viral fusion protein.

Aspect 52. The viral vector according to aspect 51, wherein the ratio of non-retargeted attachment protein sequences or envelope displayed proteins to retargeted attachment protein sequences or envelope displayed proteins is within the range of 0:1 to 1:0, but is neither 0:1 or 1:0, such as within the range of from 0.95:0.05 to 0.05:0.95, such as 0.125:0.875, 0.25:0.75, 0.375:0.625, 0.5:0.5, or 0.75:0.25.

Aspect 53. Use of the viral vector production system to any one of aspects 1 to 45, or the cell according to aspect 46 or aspect 47, for producing a viral vector.

Aspect 54. Use of the viral vector production system to any one of aspects 15 to 45, or a cell comprising the same, for producing a viral vector.

Aspect 55. A viral vector produced by the use according to aspect 54.

Aspect 56. The viral vector according to aspect 55, wherein the viral vector envelope comprises:
(ii) a non-retargeted attachment protein; and
(iii) a retargeted attachment protein; and, optionally
(iii) a viral fusion protein.

Aspect 57. The viral vector according to aspect 56, wherein the ratio of non-retargeted viral attachment protein sequences or envelope displayed proteins to retargeted viral attachment protein sequences or envelope displayed proteins is within the range of 0:1 to 1:0, but is neither 0:1 or 1:0, such as within the range of from 0.95:0.05 to 0.05:0.95, such as 0.125:0.875, 0.25:0.75, 0.375:0.625, 0.5:0.5, or 0.75:0.25.

Aspect 58. The viral vector production system according to any one of aspects 1 to 45, the method of aspects 48 and 49, and the viral vectors of aspects 50 to 52 and 55 to 57, wherein the viral vector is a lentiviral vector.

The invention will now be further described by way of Examples, which are meant to serve to assist one of ordinary skill in the art in carrying out the invention and are not intended in any way to limit the scope of the invention.

### Examples

### Materials and Methods

### Cell Culture Conditions

HEK293 Ts (HEK293 Ts) suspension cells were grown in FreeStyle™ 293 Expression Medium (Gibco) supplemented with 0.1% of Cholesterol Lipid Concentrate (Gibco) and incubated at 37° C. in 5% CO2, in a shaking incubator (25 mm orbit set at 190 RPM).

### Transfection

Cells were seeded in serum-free media and were incubated at 37° C. in 5% CO2, shaking, throughout vector production. Approximately 24 hours after seeding the cells were transfected. 23 mL flasks were transfected with 21.85 μg of genome, 2.3 μg of Gag/pol, and 1.38 μg of Rev plasmids. The remaining plasmid(s) encoding envelope proteins were transfected in varying mass ratios to a total mass of 1.61 μg. Envelope plasmids used were: VSV-G; NiV GmΔ34h (non-retargeted receptor-blinded NiV G); NiV GmΔ34 h_ScFv (receptor-blinded NIV G retargeted with an anti CD8 ScFv); NiV GmΔ34 h_DARPin (receptor-blinded NiV G retargeted with an anti CD8 DARPin); receptor blinded VSV-G K47Q and NiV FΔ22a.

NIV F (FΔ22a) was used at 1.15 μg. NiV G plasmid combinations (GmΔ34 h-GmΔ34 h_ScFv or GmΔ34 h-GmΔ34 h_DARPin were used at the indicated mass ratios to a total concentration of 0.46 ug. VSV-G K47Q was used at 1.84 ug.

Transfection was mediated by combining DNA with a Lipofectamine 2000CD and FreeStyle™ 293 Expression Medium mixture.

Sodium butyrate (Sigma) was added ~18 hrs later to 10 mM final concentration. Typically, vector supernatant was harvested 20-24 hours later, cells were pelleted by centrifugation at 1000 rpm for 5 minutes. Vectors and supernatants were aspirated from cell pellet and filtered through 0.45 μm filters.

Vectors were concentrated by overnight centrifugation using Nalgene Oak Ridge tubes (6000 xg for 18 hours at 4° C.). A 20% sucrose cushion was used in order to reduce the shear forces and preserve the integrity of the vector particles. Following supernatant removal, pellets were resuspended in TSSM.

### Preparation of PBMCs and Transduction

Peripheral blood mononuclear cells (PBMCs) were thawed from −150° C. storage into RPMI media supplemented with 5% human serum and 1% L-glutamine (cRPMI). The cells were washed twice and pelleted by centrifugation for 5 minutes at 400 xg and re-suspended in a 10 mL cell suspension. The cells were diluted to a working concentration of 1×10⁶/mL media with IL-7 and IL-15 (final concentration of 10 ng/ml each).

Human T activator beads (11132D) were washed 3 times in wash buffer (PBS with 10% RPMI media) and added to the cells using a 1:1 bead to cell ratio.

72 h after activation cells were pelleted by centrifugation at 300×g for 5 minutes then resuspended in cRPMI, counted and seeded in a 48well plate at 1.95×10⁵ cells/well in 250 μL.

The vectors were diluted 1:12.5 in cRPMI, and 250 μL of each vector dilution was added in duplicate to the appropriate well containing 250 μL cells making the final vector dilution 1:25 After vector addition, IL-7 and IL-15 were added to account for the increase in volume in each well. The plates were then incubated at 37° C. for 3 days.

For maintaining transduced PBMCs, cells were observed regularly under the microscope and samples were taken for a cell count monitoring. Cells from selected wells were resupended, counted and passaged using media containing IL-7 and IL-15 to maintain a concentration of 1E6 cells/mL.

12 days after transduction cells were analysed by flow cytometry: for staining, approximately 5E5 cells from each condition were transferred to a 96U bottom plate and pelleted for 5 minutes at 300 xg before being washed once with stain buffer. Cells were then resuspended in stain buffer with anti-CD8 PE-Cy7 antibody (Biolegend, 344712) diluted 1:100 and incubated for 20 minutes at room temperature. Cells were then washed twice with stain buffer and resuspended in 200 μL stain buffer with SYTOX™ AADvanced™ Dead Cell Stain (Thermo, S10349) diluted 1:1000. The samples and controls were run on a 2-laser Attune flow cytometer.

### Example 1: Evaluation of Transduction Efficiency of Vectors Pseudotyped with Different Ratios of Envelope Proteins

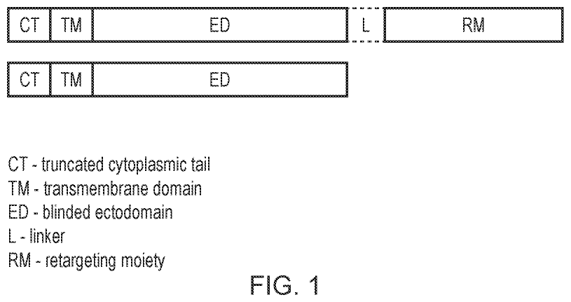

Evaluation of transduction efficiency of vectors pseudotyped with different ratios of receptor-blinded non-retargeted Nipah virus attachment protein (GmΔ34h) and receptor-blinded retargeted Nipah virus attachment protein, wherein the protein is either retargeted using an scFv or a DARPin with specificity for CD8. A schematic representation of retargeted and non-retargeted Nipah viral attachment proteins is shown in FIG. 1.

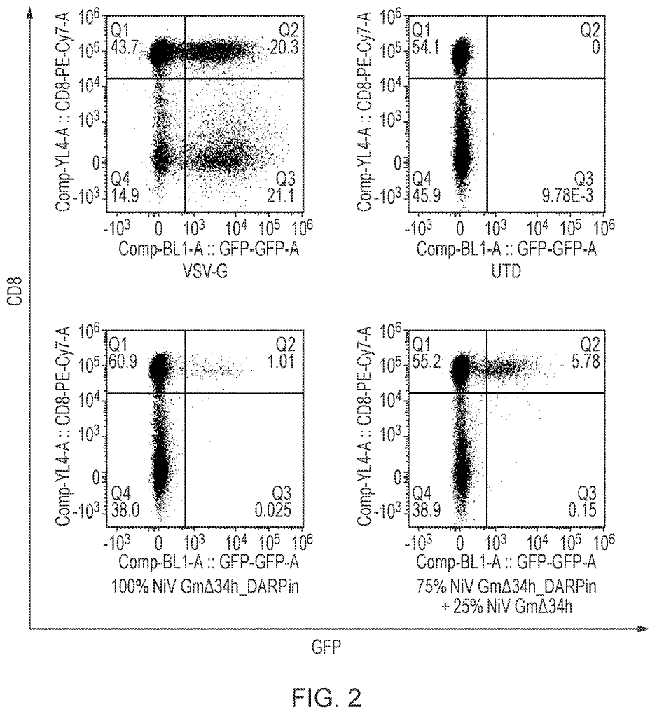

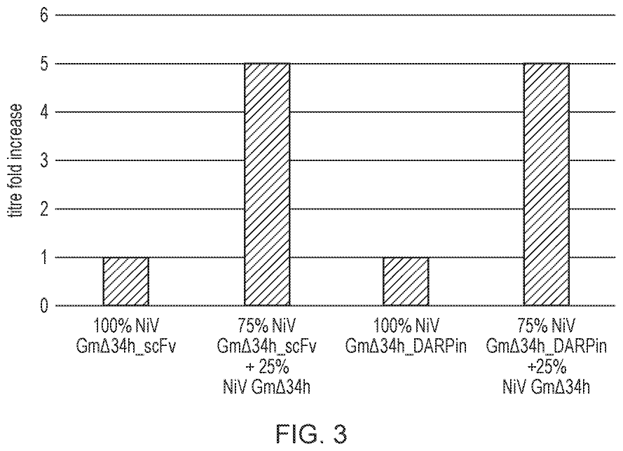

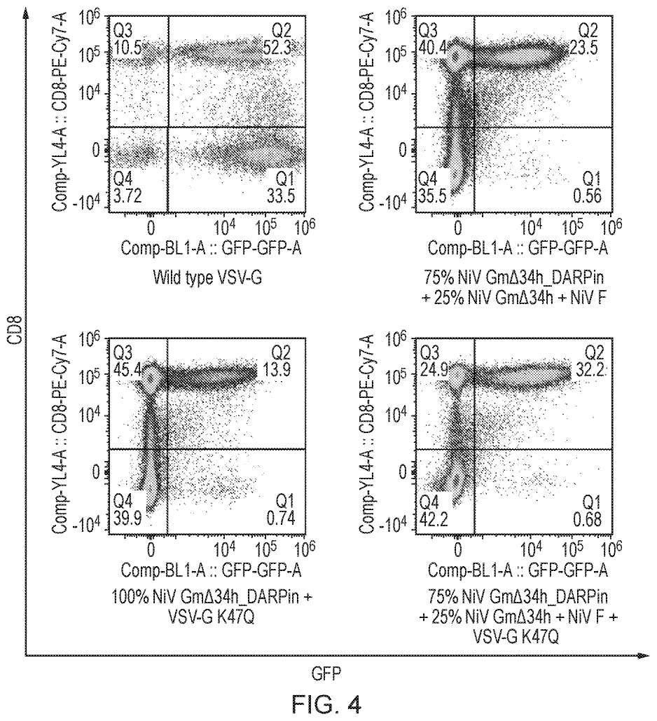

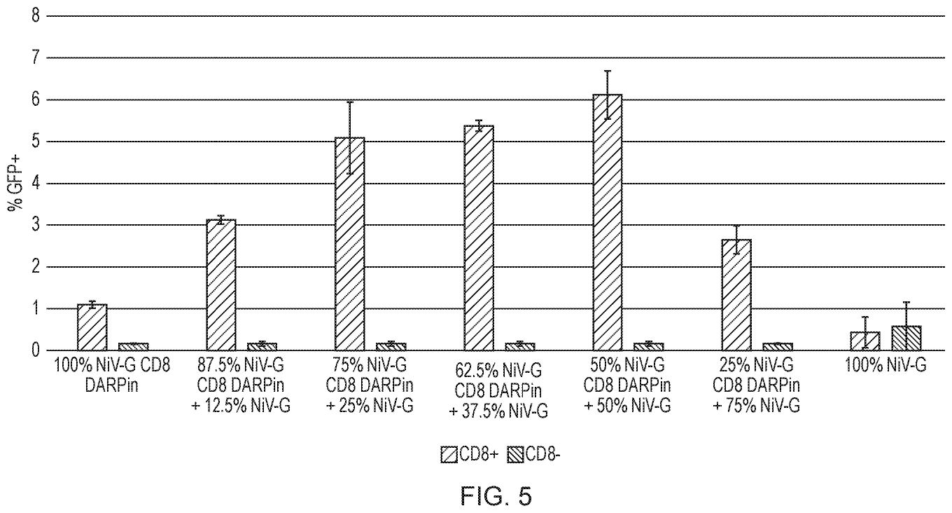

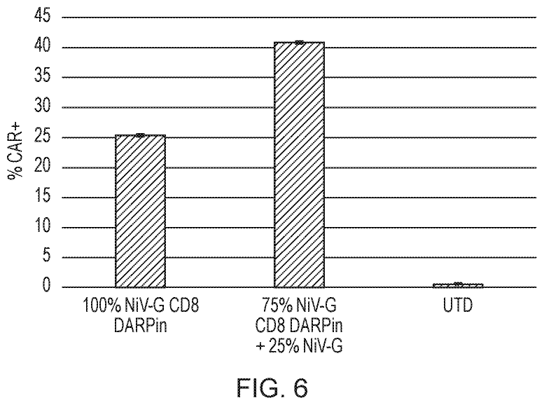

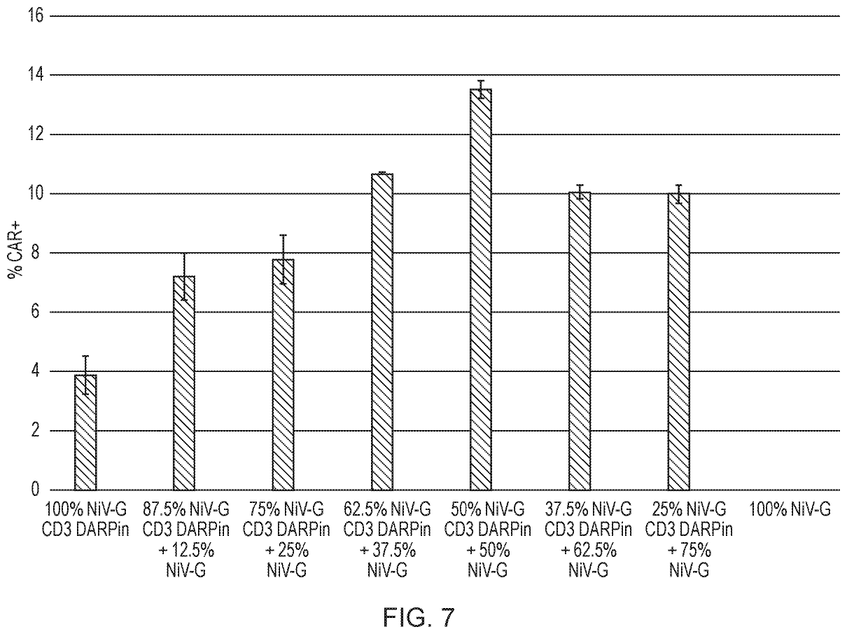

As assessed by flow cytometry, retargeted NiV envelope protein containing vectors showed high specificity for CD8+ cells, whereas vectors pseudotyped with VSV-G transduced both CD8+ and CD8-cells, indicative of a lack of target specificity (FIG. 2). FIG. 3 shows the fold increase of the biological (GFP) titre, which was calculated based on the percentage of GFP+ cells among the CD8+ population measured at day 12, and accounting for the absolute number of CD8+ cells present at transduction. The 75% NiV GmΔ34 h_DARPin (receptor-blinded NiV G retargeted with an anti CD8 DARPin)+25% NiV GmΔ34h (non-retargeted receptor-blinded NiV G) showed approximately 5-fold higher titre than the vector comprising a non-mixed envelope of just the retargeted NiV-G (100% NiV GmΔ34 h_DARPin). Similarly, the vector corresponding to 75% NiV GmΔ34 h_ScFv (receptor-blinded NiV G retargeted with an anti CD8 ScFv)+25% NiV GmΔ34h (non-retargeted receptor-blinded NiV G) had a 5-fold titre increase compared to a non-mixed envelope of just the corresponding retargeted NiV-G (100% NiV GmΔ34 h_ScFv). These results demonstrate the benefit of mixed envelopes within each class of envelope type (i.e., scFv retargeted or DARPin retargeted).

Transduction efficiencies of vectors pseudotyped with different ratios of receptor-blinded non-retargeted Nipah virus attachment protein (GmΔ34h) and receptor-blinded retargeted Nipah virus attachment protein, wherein the protein is retargeted using a DARPin with specificity for CD8 were further investigated. FIG. 5 shows the percentage of GFP+ cells among the CD8+ population measured at day 12. As assessed by flow cytometry, the highest transduction efficiency was observed with ratios 0.75:0.25, 0.625:0.375 and 0.50:0.50 (i.e. 75% NiV-GmΔ34 h_DARPin+25% NiV GmΔ34h, 62.5% NiV-GmΔ34 h_DARPin+37.5% NiV GmΔ34h, and 50% NiV-GmΔ34 h_DARPin+50% NiV GmΔ34h), showing a 5-fold improvement compared to the 1.00:0.00 ratio (containing only 100% NiV-GmΔ34 h CD8 DARPin) (FIG. 5).

In addition, transduction efficiencies were investigated by flow cytometry for CD8 targeting vectors expressing FMC63 CAR as the transgene and pseudotyped with different ratios of envelope proteins. FIG. 6 shows the percentage of CAR+ cells amongst the CD8+ T cell population measured at day 10 post transduction. The vectors pseudotyped with a mixed envelope composition of 0.75:0.25 retargeted NiV-GmΔ34 h protein to non-retargeted GmΔ34h (75% NiV-GmΔ34 h_DARPin+25% NiV GmΔ34h) exhibited improved transduction efficiency over non-mixed envelopes utilising the same type of retargeted NiV-GmΔ34h protein (FIG. 6).

Furthermore, transduction efficiencies were investigated by flow cytometry for CD3 targeting vectors expressing FMC63 CAR as the transgene and pseudotyped with different ratios of mixed and non-mixed envelope proteins. FIG. 7 shows the percentage of FMC63 CAR+ cells amongst the CD3+ T cell population measured at day 12 post transduction. The highest transduction efficiency was observed with ratio 0.50:0.50 (i.e. 50% NiV-GmΔ34 h_DARPin+50% NiV GmΔ34h), showing a 3.5-fold improvement compared to the 1.00:0.00 ratio (containing only 100% NiV-GmΔ34 h CD3 DARPin).

These results again demonstrate the benefit of mixed envelopes and the benefit of mixed envelopes in vectors carrying a CAR transgene and for retargeting to different target antigens (CD8 and CD3).

### Example 2: Evaluation of the Transduction Efficiency of Vectors Pseudotyped with a Combination of the Retargeted NiV-G, Non-Retargeted NiV-G and VSV-G K47Q Envelope Glycoproteins

Evaluation of transduction efficiency of vectors pseudotyped with a combination of receptor-blinded non-retargeted Nipah virus attachment protein (GmΔ34h), receptor-blinded retargeted Nipah virus attachment protein retargeted using a DARPin with specificity for CD8 (GmΔ34 h_DARPin), and receptor-blinded VSV-G K47Q envelope glycoprotein.

As assessed by flow cytometry, retargeted NiV G and VSV-G K47Q envelope protein containing vectors showed high specificity for CD8+ cells, whereas vectors pseudotyped with wild-type VSV-G transduced both CD8+ and CD8-cells, indicative of a lack of target specificity (FIG. 4). The combination of 75% NiV GmΔ34 h_DARPin (receptor-blinded NiV G retargeted with an anti CD8 DARPin)+25% NiV GmΔ34h (non-retargeted receptor-blinded NiV G)+VSV-G K47Q+NiV F showed higher efficiency of specific transduction (32.2% CD8+GFP+ cells-FIG. 4 Row 2 Panel 2) when compared to 75% NiV GmΔ34 h_DARPin+25% NiV GmΔ34h+NiV F (23.5% CD8+GFP+ cells-FIG. 4 Row 1 Panel 2) and 100% NiV GmΔ34 h_DARPin+VSV-G K47Q (13.9% CD8+GFP+ cells-FIG. 4 Row 2 Panel 1).

1. A viral vector production system comprising a set of nucleotide sequences, wherein the nucleotide sequences encode heterologous envelope protein sequences comprising at least one viral attachment protein, wherein the nucleotide sequences separately encode:
(i) a non-retargeted viral attachment protein; and
(ii) a retargeted viral attachment protein.

2. The viral vector production system according to claim 1, wherein the attachment protein is receptor-blinded such that it does not bind to one or more of its endogenous receptors.

3. The viral vector production system according to claim 1 or claim 2, wherein the non-retargeted attachment protein and retargeted attachment protein are derived from the same virus.

4. The viral vector production system according to any one of claims 1 to 3, wherein the viral attachment protein is a negative sense RNA virus attachment protein, or a modified version thereof.

5. The viral vector production system according to any one of claims 1 to 4, wherein the viral attachment protein is a mononegavirus attachment protein, or a modified version thereof.

6. The viral vector production system according any one of claims 1 to 5, wherein the viral attachment protein is a paramyxovirus attachment protein, or a modified version thereof.

7. The viral vector production system according to any one of claims 1 to 6, wherein the viral attachment protein is a henipavirus attachment protein, or a modified version thereof.

8. The viral vector production system according to any one of claims 1 to 7, wherein the viral attachment protein is a Nipah virus attachment protein, or a modified version thereof.

9. The viral vector production system according to any one of claims 1 to 8, wherein the viral attachment protein:
(a) is a mA34 mutant of the Nipah virus attachment protein; and/or
(b) comprises a sequence of SEQ ID NO: 1 or SEQ ID NO: 2.

10. The viral vector production system according to any one of claims 1 to 5, wherein the viral attachment protein is a rhabdovirus attachment protein, or a modified version thereof.

11. The viral vector production system according to any one of claims 1 to 5, and claim 10, wherein the viral attachment protein is a Vesicular stomatitis virus (VSV) protein, or a modified version thereof.

12. The viral vector production system according to any one of claims 1 to 5, and claims 10 and 11, wherein the viral attachment protein:
(a) is a K47Q mutant of VSV-G;
(b) is a R354A mutant of VSV-G; and/or
(c) comprises a sequence of SEQ ID NO: 34 or SEQ ID NO: 35.

13. The viral vector production system according to any one of claims 1 to 12, wherein the retargeted viral attachment protein comprises a retargeting moiety.

14. The viral vector production system according to any one of claims 1 to 13, wherein the retargeting moiety is a protein.

15. The viral vector production system according to any one of claims 1 to 14, wherein the retargeting moiety is a protein selected from the group consisting of: an antibody, an scFV, a nanobody, a DARPin, and a glycoprotein.

16. The viral vector production system according to any one of claims 1 to 15, wherein the retargeting moiety has affinity for CD8 or CD3.

17. The viral vector production system according to claim 16, wherein the retargeting moiety has affinity for CD8.

18. The viral vector production system according to claim 16, wherein the retargeting moiety has affinity for CD3.

19. The viral vector production system according to any one of claims 1 to 18, wherein the retargeted attachment protein is a chimeric protein.

20. The viral vector production system according to any one of claims 1 to 19, wherein the nucleotide sequences encode a viral fusion protein.

21. The viral vector production system according to any one of claims 1 to 20, wherein the viral fusion protein is a negative sense RNA virus fusion protein or modified version thereof.

22. The viral vector production system according to any one of claims 1 to 21, wherein the viral fusion protein is a mononegavirus fusion protein or modified version thereof.

23. The viral vector production system according to any one of claims 1 to 22, wherein the viral fusion protein is a paramyxovirus fusion protein or modified version thereof.

24. The viral vector production system according to any one of claims 1 to 23, wherein the viral fusion protein is a henipavirus fusion protein or modified version thereof.

25. The viral vector production system according to any one of claims 1 to 24, wherein the viral fusion protein is a Nipah virus fusion protein or modified version thereof.

26. The viral vector production system according to any one of claims 1 to 25, wherein the viral fusion protein:
(a) is a Δ22 mutant of the Nipah virus fusion protein; and/or
(b) comprises a sequence of SEQ ID NO: 3 or SEQ ID NO: 4.

27. The viral vector production system according to any one of claims 1 to 22, wherein the viral attachment protein is a rhabdovirus fusion protein, or a modified version thereof.

28. The viral vector production system according to any one of claims 1 to 22, and claim 27, wherein the viral fusion protein is a Vesicular stomatitis virus (VSV) protein, or a modified version thereof.

29. The viral vector production system according to any one of claims 1 to 22, and claims 27 and 28, wherein the viral fusion protein:
(a) is a K47Q mutant of VSV-G;
(b) is a R354A mutant of VSV-G; and/or
(c) comprises a sequence of SEQ ID NO: 34 or SEQ ID NO: 35.

30. The viral vector production system according to any one of claims 1 to 29, wherein the ratio of non-retargeted viral attachment protein sequences to retargeted viral attachment protein sequences is within the range of 0.05:0.95 to 0.95:0.05, optionally within the range of from 0.125:0.875 to 0.75:0.25.

31. The viral vector production system according to any one of claims 1 to 30, wherein the ratio of non-retargeted viral attachment protein sequences to retargeted viral attachment protein sequences is 0.125:0.875, 0.25:0.75, 0.375:0.625, 0.5:0.5, or 0.75:0.25.

32. A cell comprising the viral vector production system according to any one of claims 1 to 31.

33. A cell for producing viral vectors comprising the viral vector production system according to any one of claims 1 to 31.

34. A method for producing a viral vector, comprising the steps of:
(a) introducing the viral vector production system according to any one of claims 1 to 31 into a cell;
(b) optionally, selecting for a cell which comprises the nucleotide sequences of the viral vector production system according to any one of claims 1 to 31; and
(c) culturing the cell under conditions suitable for the production of the viral vector.

35. A viral vector produced by the method according to claim 34.

36. The viral vector produced by the method according to claim 34, wherein the viral vector envelope comprises:
(i) a non-retargeted attachment protein; and
(ii) a retargeted attachment protein; and, optionally (iii) a viral fusion protein.

37. The viral vector according to claim 36, wherein the ratio of non-retargeted attachment protein sequences or envelope displayed proteins to retargeted attachment protein sequences or envelope displayed proteins is within the range of from 0.05:0.95 to 0.95:0.05, such as, 1.125:0.875, 0.25:0.75, 0.375:0.625, 0.5:0.5, or 0.75:0.25.

38. Use of the viral vector production system to any one of claims 1 to 31, or the cell according to claim 32 or claim 33, for producing a viral vector.

39. A viral vector produced by the use according to claim 38.

40. The viral vector according to claim 39, wherein the viral vector envelope comprises:
(ii) a non-retargeted attachment protein; and
(iii) a retargeted attachment protein; and, optionally
(iii) a viral fusion protein.

41. The viral vector according to claim 40, wherein the ratio of non-retargeted viral attachment protein sequences or envelope displayed proteins to retargeted viral attachment protein sequences or envelope displayed proteins is within the range of 0.05:0.95 to 0.95:0.05, such as, 0.125:0.875, 0.25:0.75, 0.375:0.625, 0.5:0.5, or 0.75:0.25.

42. The viral vector production system according to any one of claims 1 to 31, the method of claim 34, and the viral vectors of claims 35 to 37 and 39 to 41, wherein the viral vector is a lentiviral vector.


SEQUENCE LISTING (40 sequences)

SEQ ID NO: 1 - protein, 569 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 2 - protein, 575 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTHHHHHH

SEQ ID NO: 3 - protein, 524 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 4 - protein, 530 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTDTYRYI

SEQ ID NO: 5 - protein, 240 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIK

SEQ ID NO: 6 - protein, 157 aa, synthetic construct
DLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAA

SEQ ID NO: 7 - protein, 741 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAA

SEQ ID NO: 8 - protein, 829 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSAAQPAQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIK

SEQ ID NO: 9 - DNA, 30 bp, synthetic construct
  source 1..30: mol_type = other DNA
ggggcggcgactggtgagtacgccaaaaat

SEQ ID NO: 10 - DNA, 30 bp, synthetic construct
  source 1..30: mol_type = other DNA
ggggcggcgactgcagacaacgccaaaaat

SEQ ID NO: 11 - DNA, 30 bp, synthetic construct
  source 1..30: mol_type = other DNA
ggggcggcgagtggagactacgccaaaaat

SEQ ID NO: 12 - DNA, 32 bp, synthetic construct
  source 1..32: mol_type = other DNA
ggggaaggcaacagataaatatgccttaaaat

SEQ ID NO: 13 - DNA, 19 bp, synthetic construct
  source 1..19: mol_type = other DNA
ggcgactggtgagtacgcc

SEQ ID NO: 14 - DNA, 19 bp, synthetic construct
  source 1..19: mol_type = other DNA
ggcgactgcagacaacgcc

SEQ ID NO: 15 - DNA, 19 bp, synthetic construct
  source 1..19: mol_type = other DNA
ggcgagtggagactacgcc

SEQ ID NO: 16 - DNA, 23 bp, synthetic construct
  source 1..23: mol_type = other DNA
aaggcaacagataaatatgcctt

SEQ ID NO: 17 - DNA, 339 bp, synthetic construct
  source 1..339: mol_type = other DNA
atgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagagggagagcaaaacaaaagtaaga

SEQ ID NO: 18 - DNA, 81 bp, synthetic construct
  source 1..81: mol_type = other DNA
atgggtgcgagagcgtcagtattaagcgggggagaattagatcgcgatgggaaaaaattcggttaaggccagggggaaaga

SEQ ID NO: 19 - DNA, 341 bp, synthetic construct
  source 1..341: mol_type = other DNA
acgggtgcgagagcgtcagtattaagcgggggagaattagatcgcgattgggaaaaaattcggttaaggccagggggaaagaaaaaatataaattaaaacatatagtattgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagagggagagcaaaacaaaagtaaga

SEQ ID NO: 20 - DNA, 82 bp, synthetic construct
  source 1..82: mol_type = other DNA
acgggtgcgagagcgtcagtattaagcgggggagaattagatcgcgattgggaaaaaattcggttaaggccagggggaaaga

SEQ ID NO: 21 - DNA, 255 bp, synthetic construct
  source 1..255: mol_type = other DNA
aaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagagggagagcaaaacaaaagta

SEQ ID NO: 22 - DNA, 234 bp, synthetic construct
  source 1..234: mol_type = other DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 23 - DNA, 351 bp, synthetic construct
  source 1..351: mol_type = other DNA
ggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggac

SEQ ID NO: 24 - DNA, 783 bp, synthetic construct
  source 1..783: mol_type = other DNA
tgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggac

SEQ ID NO: 25 - DNA, 236 bp, synthetic construct
  source 1..236: mol_type = other DNA
aggagctttgttccttgggttcttgggagcagcaggaagcactattgggcgcagcgtcaattgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 26 - DNA, 355 bp, synthetic construct
  source 1..355: mol_type = other DNA
ggagctttgttccttgggttcttgggagcagcaggaagcactattgggcgcagcgtcaattgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaattgctagttggagtaataaatctctggaacagatttggaatcacacgacctggattggagtgggac

SEQ ID NO: 27 - DNA, 789 bp, synthetic construct
  source 1..789: mol_type = other DNA
tgatcttcagacctggaggaggagatattgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactattgggcgcagcgtcaattgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaattgctagttggagtaataaatctctggaacagatttggaatcacacgacctggattggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataattgatagtaggaggcttggtaggtttaagaatagtttttgctgtactttctatagtgaatagagttaggcagggatattcaccattatcgtttcagacccacctcccaaccccgaggggac

SEQ ID NO: 28 - DNA, 791 bp, synthetic construct
  source 1..791: mol_type = other DNA
tgatcttcagacctggaggaggagatattgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactattgggcgcagcgtcaattgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaattgctagttggagtaataaatctctggaacagatttggaatcacacgacctggattggagtgggacagagaaattaacaattacacaagcttaatacactccttaattgaagaatcgcaaaaccagcaagaaaagaattgaacaagaattattggaattagataaattgggcaagtttgtggaattggtttaacataacaaattggctgtggtatataaaattattcataattgatagtaggaggcttggtaggtttaagaatagtttttgctgtactttctatagtgaatagagttaggcagggatattcaccattatcgtttcagacccacctcccaaccccgaggggac

SEQ ID NO: 29 - DNA, 585 bp, synthetic construct
  source 1..585: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 30 - DNA, 585 bp, synthetic construct
  source 1..585: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 31 - DNA, 482 bp, synthetic construct
  source 1..482: mol_type = other DNA
atgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 32 - DNA, 587 bp, synthetic construct
  source 1..587: mol_type = other DNA
aatcaacctctggattacaaaatttgtgaaagattgactggtattcttaactatgttgctccttttacgctatgtggatacgctgctttaatgcctttgtatcattgctattgcttcccgtatggctttcattttctcctccttgtataaatcctggttgctgtctctttattgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 33 - DNA, 591 bp, synthetic construct
  source 1..591: mol_type = other DNA
aatcaacctctggattacaaaatttgtgaaagattgactggtattcttaactattgttgctccttttacgctattgtggatacgctgctttaattgcctttgtatcattgctattgcttcccgtattggctttcattttctcctccttgtataaatcctggttgctgtctctttattgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 34 - protein, 511 aa, synthetic construct
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 35 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 36 - protein, 752 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAAGGGSGHHHHHH

SEQ ID NO: 37 - protein, 842 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSAAQPAQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRAAARGSHHHHHH

SEQ ID NO: 38 - protein, 124 aa, synthetic construct
DLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAA

SEQ ID NO: 39 - protein, 708 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSDLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAA

SEQ ID NO: 40 - protein, 719 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSDLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAAGGGSGHHHHHH